(19) United States
(12) Patent Application Publication
(10) Pub. No.: US 2009/0275994 A1
(43) Pub. Date: Nov. 5, 2009

(54) APPARATUS AND METHODS FOR INSERTING FACET SCREWS

(76) Inventors: Christopher U. PHAN, San Leandro, CA (US); Hugues F. Malandain, Mountain View, CA (US)

Correspondence Address: COOLEY GODWARD KRONISH LLP;ATTN: PATENT GROUP, Suite 1100, 777 - 6th Street, NW, WASHINGTON, DC, 20001, US

(21) Appl. No.: 12/112,701

(22) Filed: Apr. 30, 2008

(51) Int. Cl. A61F 5/00; B25B 13/00; A61B 17/58

(52) U.S. Cl. 606/86 A; 81/52; 606/103

(57) ABSTRACT

A method includes inserting a bone fixation device into a patient's body. The bone fixation device includes a first member and a second member movably coupled to the first member. A passageway is defined within a bone tissue after the bone fixation device is inserted and while the bone fixation device is disposed within the patient's body. At least a portion of the first member of the bone fixation device is disposed within the bone tissue along the passageway. The second member of the bone fixation device is moved relative to the first member of the bone fixation device.

### CROSS-REFERENCE TO RELATED APPLICATIONS

This application is related to U.S. patent application Attorney Docket Nos. KYPH-037/00US 305363-2161, KYPH-037/01US 305363-2260 and KYPH-037/03US 305363-2261, and, each entitled “Apparatus and Methods for Inserting Facet Screws,” filed on the same date, each of which is incorporated herein by reference in their entirety.

### BACKGROUND

The invention relates generally to medical devices and procedures. More particularly, the invention relates to apparatus and methods for inserting screws into bone tissue.

Bone fixation devices, such as, for example, bone screws, staples, and/or clamping mechanisms, can be used in various medical procedures. For example, known bone screws can be used to repair fractured bone tissue by clamping adjacent portions of the bone tissue together. Known bone screws can also be used to stabilize and/or limit the movement of bone tissue. For example, some known bone screws can be used as a part of a spinal fixation procedure.

In some procedures, for example, a facet screw can be inserted across the facet joint of the spinal column to fuse and/or limit the motion of the facet joint. Such known procedures can include, for example, translaminar facet screw fixation, which includes inserting a facet screw from the base of the spinous process on the contralateral side and through the lamina to traverse the facet joint in a plane perpendicular to the joint surfaces. Facet screws can also be inserted using a transfacet approach, which involves inserting a bone screw via a midline incision or an ipsilateral incision. Such known procedures can further include threadedly coupling a nut to the proximal end of the facet screw to fuse the facet joint. Such known procedures, however, often involve the use of multiple tools and/or multiple steps. For example, such known procedures can include separate steps and tools to advance a guide wire into the targeted bone tissue, insert the facet screw into the targeted bone tissue, and/or couple the nut to the proximal end of the facet screw.

Thus, a need exists for improved insertion tools, bone fixations devices, and procedures for inserting facet screws into bone tissue.

### SUMMARY

Apparatus and methods for inserting facet screws are described herein. In some embodiments, a method includes inserting a bone fixation device into a patient's body. The bone fixation device includes a first member and a second member movably coupled to the first member. A passageway is defined within a bone tissue after the bone fixation device is inserted and while the bone fixation device is disposed within the patient's body. At least a portion of the first member of the bone fixation device is disposed within the bone tissue along the passageway. The second member of the bone fixation device is moved relative to the first member of the bone fixation device.

### BRIEF DESCRIPTION OF THE DRAWINGS

FIG. 1 is a schematic illustration of a medical device according to an embodiment of the invention.

FIGS. 2 and 3 are schematic illustrations of a medical device according to an embodiment of the invention in a first configuration and a second configuration, respectively.

FIG. 4 is a front view of a medical device according to an embodiment of the invention.

FIG. 5 is a cross-sectional view of the medical device shown in FIG. 4 taken along line X-X.

FIG. 6 is a schematic illustration of a medical device according to an embodiment of the invention.

FIG. 7 is a perspective view of a medical device according to an embodiment of the invention.

FIG. 8 is an exploded perspective view of the medical device shown in FIG. 7.

FIG. 9 is a front view of the medical device shown in FIG. 7.

FIG. 10 is a cross-sectional view of the medical device shown in FIG. 7 taken along line X-X in FIG. 9.

FIGS. 11 and 12 are perspective views of a portion of the medical device shown in FIG. 7.

FIG. 13 is a proximal side view of the portion of the medical device shown in FIGS. 11 and 12.

FIG. 14 is a cross-sectional view of the portion of the medical device shown in FIG. 13 taken along line X-X in FIG. 13.

FIG. 15 is a distal side view of the portion of the medical device shown in FIGS. 11 and 12.

FIG. 16 is a cross-sectional view of the portion of the medical device shown in FIG. 15 taken along line X-X in FIG. 15.

FIG. 17 is a front view of a portion of the medical device shown in FIG. 7.

FIG. 18 is a distal side view of the portion of the medical device shown in FIG. 17.

FIG. 19 is a proximal side view of the portion of the medical device shown in FIG. 17.

FIG. 20 is an exploded perspective view of a portion of the medical device shown in FIG. 7.

FIG. 21 is an front view of the portion of the medical device shown in FIG. 20.

FIG. 22 is a cross-sectional view of the portion of the medical device shown in FIG. 21 taken along line X-X in FIG. 21.

FIGS. 23 and 24 are cross-sectional views of the portion of the medical device shown in FIG. 22 marked as region Z, with the medical device in a first configuration and a second configuration, respectively.

FIG. 25 is an exploded perspective view of a portion of the medical device shown in FIG. 7.

FIGS. 26 and 27 are perspective views of the portion of the medical device shown in FIG. 25 in a first configuration and a second configuration, respectively.

FIG. 28 is a perspective view of a portion of the medical device shown in FIG. 7.

FIG. 29 is a bottom view of the portion of the medical device shown in FIG. 28.

FIG. 30 is a cross-sectional view of the portion of the medical device shown in FIG. 29 taken along line X-X in FIG. 29.

FIG. 31 is an exploded perspective view of a portion of the medical device shown in FIG. 7.

FIG. 32 is a perspective view of a portion of the medical device shown in FIG. 7.

FIG. 33 is a top view of the portion of the medical device shown in FIG. 32.

FIG. 34 is a cross-sectional view of the portion of the medical device shown in FIG. 33 taken along line X-X in FIG. 33.

FIG. 35 is an exploded perspective view of a portion of the medical device shown in FIG. 7.

FIG. 36 is an exploded perspective view of a portion of the medical device shown in FIG. 7.

FIG. 37 is a front view of a bone fixation device according to an embodiment of the invention.

FIG. 38 is a cross-sectional view of the bone fixation device shown in FIG. 37 taken along line X-X in FIG. 37.

FIGS. 39 and 40 are a front view and a top view, respectively, of a portion of the bone fixation device shown in FIG. 37.

FIG. 41 is a cross-sectional view of the portion of the bone fixation device shown in FIG. 40 taken along line X-X in FIG. 40.

FIGS. 42 and 43 are a front view and a top view, respectively, of a portion of the bone fixation device shown in FIG. 37.

FIG. 44 is a cross-sectional view of the portion of the bone fixation device shown in FIG. 42 taken along line X-X in FIG. 42.

FIG. 45 is a cross-sectional view of a portion of the bone fixation device shown in FIG. 37.

FIGS. 46 and 47 are front views of a portion of the bone fixation device shown in FIG. 37, in a first configuration and a second configuration, respectively.

FIGS. 48 through 54 are views showing a method of inserting the bone fixation device shown in FIG. 37 into a portion of the spine S using the medical device shown in FIG. 7.

FIG. 55 is a flow chart of a method according to an embodiment of the invention.

FIG. 56 is a flow chart of a method according to an embodiment of the invention.

FIG. 57 is a flow chart of a method according to an embodiment of the invention.

FIG. 58 is a perspective view of a medical device according to an embodiment of the invention.

FIG. 59 is an exploded perspective view of the medical device shown in FIG. 58.

FIG. 60 is a cross-sectional view of the proximal portion of the medical device shown in FIG. 58 taken along line X-X in FIG. 58.

FIG. 61 is a cross-sectional view of the distal portion of the medical device shown in FIG. 58 in a first configuration, taken along line X-X in FIG. 58.

FIG. 62 is a perspective view of the distal portion of the medical device shown in FIG. 58.

FIG. 63 is a cross-sectional view of the distal portion of the medical device shown in FIG. 58 in a second configuration, taken along line X-X in FIG. 58.

FIG. 64 is a perspective view of a bone fixation device according to an embodiment of the invention.

FIG. 65 is a cross-sectional view of the bone fixation device shown in FIG. 64.

FIG. 66 is an exploded view of the bone fixation device shown in FIG. 64.

FIG. 67 is a perspective view of a medical device according to an embodiment of the invention.

FIG. 68 is an exploded perspective view of the medical device shown in FIG. 67.

FIG. 69 is a cross-sectional view of the proximal portion of the medical device shown in FIG. 67 taken along line X-X in FIG. 67.

FIG. 70 is a cross-sectional view of the distal portion of the medical device shown in FIG. 67 in a first configuration, taken along line X-X in FIG. 67.

FIG. 71 is a perspective view of the first shaft of the medical device shown in FIG. 67.

FIG. 72 is a cross-sectional view of the first shaft of the medical device shown in FIG. 71 taken along line X-X in FIG. 71.

FIG. 73 is a perspective view of the second shaft and third shaft of the medical device shown in FIG. 67.

FIG. 74 is a cross-sectional view of the second shaft and third shaft of the medical device shown in FIG. 73 taken along line X-X in FIG. 73.

FIG. 75 is a perspective view of the dial actuator of the medical device shown in FIG. 67.

FIG. 76 is a cross-sectional view of the dial actuator of the medical device shown in FIG. 75 taken along line X-X in FIG. 75.

FIG. 77 is a perspective view of a medical device according to an embodiment of the invention.

FIG. 78 is a cross-sectional view of the distal portion of the medical device shown in FIG. 77.

FIG. 79 is a perspective view of the sheath of the medical device shown in FIG. 77.

FIG. 80 is a cross-sectional view of the sheath of the medical device shown in FIG. 77 taken along line X-X in FIG. 79.

FIG. 81 is a perspective view of the retention member of the medical device shown in FIG. 77.

### DETAILED DESCRIPTION

Apparatus and methods for inserting facet screws are described herein. In some embodiments, an apparatus includes a first shaft, a second shaft, and a locking mechanism. The first shaft has a threaded portion and an engagement portion. The engagement portion of the first shaft is configured to engage a nut. The second shaft has a threaded portion and an engagement portion. The engagement portion of the second shaft is configured to engage a screw, which can be, for example, a self-tapping bone screw. At least a portion of the second shaft is disposed within the first shaft such that the threaded portion of the first shaft is engaged with the threaded portion of the second shaft. The locking mechanism is configured to selectively allow rotation of the second shaft relative to the first shaft.

In some embodiments, an apparatus includes a first shaft, a second shaft, and a locking mechanism. The first shaft has a proximal end portion, a distal end portion, and a threaded portion. The distal end portion of the first shaft includes an engagement portion configured to engage a nut. The proximal end portion of the first shaft includes an inner surface defining a recess and multiple grooves, which can be, for example, spines. The second shaft has a threaded portion and an engagement portion. The engagement portion of the second shaft is configured to engage a screw. At least a portion of the second shaft is disposed within the first shaft such that the threaded portion of the first shaft is engaged with the threaded portion of the second shaft. The locking mechanism has a first configuration and a second configuration. The locking mechanism is configured to limit the rotation of the second shaft relative to the first shaft when in the first configuration. The locking mechanism is configured to allow rotation of the second shaft relative to the first shaft when in the second configuration. The locking mechanism includes a lock housing, a biasing member and a lock tab. The lock housing is disposed about the second shaft and within the recess of the first shaft. The biasing member, which can be, for example, a spring, is disposed within the lock housing. The lock tab has a first end and a second end. At least a portion of the lock tab is movably disposed within the lock housing such that the first end of the lock tab is in contact with the biasing member and a second end of the lock tab is disposed outside of the lock housing and within a groove of the first shaft when the locking mechanism is in the first configuration.

In some embodiments, an apparatus includes a first shaft, a second shaft, and a locking mechanism. The first shaft has an engagement portion configured to engage a first rotatable member of a bone fixation device. The first rotatable member can be, for example, a nut. The second shaft has an engagement portion configured to engage a second rotatable member of the bone fixation device when the second rotatable member is coupled to the first rotatable member. The second rotatable member can be, for example, a self-tapping bone screw. At least a portion of the second shaft is disposed within and coupled to the first shaft such that the first shaft is configured to move a predetermined axial distance relative to the second shaft when the first shaft rotates about the second shaft. The locking mechanism is configured to selectively allow rotation of the first shaft about the second shaft.

In some embodiments, an apparatus includes a first shaft, a second shaft, and a locking mechanism. The first shaft has a proximal end portion and a distal end portion. The distal end portion of the first shaft is configured to engage a nut. The second shaft has a proximal end portion and a distal end portion. The distal end portion of the second shaft is configured to engage a screw. At least a portion of the distal end portion of the second shaft is disposed within the first shaft. The locking mechanism has a first configuration and a second configuration. The locking mechanism is configured to limit the rotation of the second shaft relative to the first shaft when in the first configuration. The locking mechanism is configured to allow rotation of the second shaft relative to the first shaft when in the second configuration. The locking mechanism includes a biasing member configured to bias the locking mechanism in the first configuration.

In some embodiments, an apparatus includes a first shaft, a second shaft, a locking mechanism, and an actuator. The first shaft has a proximal end portion and a distal end portion. The distal end portion of the first shaft is configured to engage a nut. The second shaft has a proximal end portion and a distal end portion. The distal end portion of the second shaft is configured to engage a screw. At least a portion of the distal end portion of the second shaft is disposed within the first shaft. The locking mechanism is configured to limit the rotation of the second shaft relative to the first shaft when the locking mechanism is in a first configuration. The locking mechanism is configured to allow the rotation of the second shaft relative to the first shaft when the locking mechanism is in a second configuration. The actuator is configured to move the locking mechanism between the first configuration and the second configuration by rotating about a longitudinal axis of the second shaft.

In some embodiments, an apparatus includes an insertion tool and a guide wire. The insertion tool has a proximal end portion and a distal end portion. The distal end portion of the insertion tool is configured to retain a bone fixation device, such as, for example, a bone screw. The proximal end portion of the insertion tool defines a threaded opening. The guide wire has a proximal end portion and a distal end portion. At least a portion of the guide wire is configured to be disposed within the insertion tool such that the distal end portion of the guide wire is disposed outside of and spaced apart from the distal end portion of the insertion tool. In some embodiments, for example, the guide wire is configured to be selectively spaced apart from the distal end portion of the insertion tool by a predetermined distance. The proximal end portion of the guide wire includes a threaded portion configured to be disposed within and engage the threaded opening of the insertion tool.

In some embodiments, an apparatus includes an apparatus includes a first shaft, a second shaft and a guide wire. The first shaft has a proximal end portion and a distal end portion. The distal end portion of the first shaft is configured to engage a nut. The second shaft has a proximal end portion and a distal end portion. The distal end portion of the second shaft is configured to engage a screw, which can, for example, be threadedly coupled to the nut. At least a portion of the distal end portion of the second shaft is disposed within the first shaft, and the first shaft is configured to rotate about the second shaft to rotate the nut about the screw. The guide wire has a proximal end portion and a distal end portion. At least a portion of the guide wire is disposed within the second shaft such that the distal end portion of the guide wire is disposed outside of and is spaced apart from the distal end portion of the second shaft.

In some embodiments, an apparatus includes an apparatus includes a first shaft, a second shaft and a guide wire. The first shaft has a proximal end portion and a distal end portion. The distal end portion of the first shaft is configured to engage a nut. The second shaft has a proximal end portion and a distal end portion. The distal end portion of the second shaft is configured to engage a screw, which can, for example, be threadedly coupled to the nut. At least a portion of the distal end portion of the second shaft is disposed within the first shaft, and the first shaft is configured to rotate about the second shaft to rotate the nut about the screw. The guide wire has a proximal end portion and a distal end portion. At least a portion of the guide wire is disposed within the second shaft such that the distal end portion of the guide wire is disposed outside of and is spaced apart from the distal end portion of the second shaft. The guide wire is movable relative to the second shaft between a first position and a second position. The distal end portion of the guide wire is spaced apart from the distal end portion of the second shaft by a first distance when the guide wire is in the first position. The distal end portion of the guide wire is spaced apart from the distal end portion of the second shaft by a second distance different than the first distance when the guide wire is in the second position.

In some embodiments, a method includes inserting percutaneously a distal end portion of an insertion tool and a bone fixation device. The bone fixation device has a proximal end portion and a distal end portion. The proximal end portion of the bone fixation device is removably coupled to the distal end portion of the insertion tool. The insertion tool includes a guide member disposed within the bone fixation device such that a distal end portion of the guide member is spaced distally from the distal end portion of the bone fixation device by a first distance. The guide member is advanced into a bone tissue by a second distance. In some embodiments, for example, the guide member can be advanced by striking a proximal end portion of the guide member with a hammer. The guide member is then moved relative to the insertion tool and the bone fixation device such that the distal end portion of the guide member is spaced distally from the distal end portion of the bone fixation device by a third distance greater than the first distance.

In some embodiments, a method includes inserting a bone fixation device into a patient's body. The bone fixation device includes a first member and a second member movably coupled to the first member. A passageway is defined within a bone tissue after the bone fixation device is inserted and while the bone fixation device is disposed within the patient's body. At least a portion of the first member of the bone fixation device is disposed within the bone tissue along the passageway. The second member of the bone fixation device is moved relative to the first member of the bone fixation device.

In some embodiments, a method includes inserting a bone fixation device into a patient's body using an insertion tool. The bone fixation device includes a first member and a second member movably coupled to the first member. The bone fixation device is coupled to the distal end portion of the insertion tool during the inserting. A passageway is defined within a bone tissue after the bone fixation device is inserted and while the bone fixation device is disposed within the patient's body. The passageway is defined by advancing a first shaft of the insertion tool into the bone tissue. At least a portion of the first member of the bone fixation device is disposed within the bone tissue along the passageway by rotating a second shaft of the insertion tool such that at least the first member of the bone fixation device is threadedly disposed within the passageway. The second member of the bone fixation device is moved relative to the first member of the bone fixation device by rotating a third shaft of the insertion tool relative to the second shaft of the insertion tool such that at least the second member of the bone fixation device rotates relative to the first member of the bone fixation device.

In some embodiments, a method includes coupling a bone fixation device to a distal end portion of an insertion tool such that distal movement of the bone fixation device along its longitudinal axis relative to the insertion tool is limited. The bone fixation device includes a first member and a second member movably coupled to the first member. At least a portion of the first member of the bone fixation device is advanced into a bone tissue within a patient's body using the insertion tool. The second member of the bone fixation device is moved relative to the first member of the bone fixation device after the portion of the first member of the bone fixation device is advanced. The second member of the bone fixation device is moved using the insertion tool. In some embodiments, the method further includes decoupling the bone fixation device from the distal end portion of the insertion tool after the second member of the bone fixation device is moved.

In some embodiments, a kit includes a bone fixation device and an insertion tool. The bone fixation device includes a bone screw and a nut threadedly coupled to the bone screw. The insertion tool is configured to define a passageway within a bone tissue within a patient's body. The insertion tool is further configured to insert at least a portion of the bone fixation device into the passageway of the bone tissue. The insertion tool includes a first shaft, a second shaft, and a third shaft. The first shaft has a distal end portion removably coupled to the nut and is configured to rotate the nut about the bone screw. The second shaft has a portion movably disposed within the first shaft. The second shaft has a distal end portion engaged with the bone screw. The second shaft is configured to rotate the bone screw. The third shaft has a portion movably disposed within the second shaft and is configured to define the passageway within the bone tissue.

In some embodiments, an apparatus includes a bone screw, a nut, and a washer. The bone screw has a first threaded portion, a second threaded portion, and a recessed portion disposed between the first threaded portion and the second threaded portion. The first threaded portion is configured to be threaded into a bone tissue. The nut is threadedly coupled to the second threaded portion of the bone screw. The nut having a tool engagement portion and a seating portion. The washer is disposed about the bone screw such that a first surface of the washer is disposed about the seating portion of the nut and a retention portion is disposed within the recessed portion of the bone screw. The washer is configured to rotate about an axis substantially normal to a longitudinal axis of the bone screw.

In some embodiments, an apparatus includes an elongate member, a retention member, and a washer. The elongate member has a proximal end portion and a distal end portion. The distal end portion of the elongate member is configured to be disposed within a bone structure. The retention member is coupled to the proximal end portion of the elongate member. The washer is disposed about the elongate member such that a proximal surface of the washer is in contact with a distal surface of the retention member. The washer is configured to move relative to the elongate member along the longitudinal axis over a predetermined range of motion. The washer is configured to rotate about an axis substantially normal to the longitudinal axis of the elongate member.

In some embodiments, an apparatus includes an elongate member, a retention member, and a washer. The elongate member has a proximal end portion and a distal end portion. The distal end portion of the elongate member is configured to be disposed within a bone structure. The retention member is coupled to the proximal end portion of the elongate member. The retention member is configured to move relative to the elongate member along a longitudinal axis of the elongate member. The retention member is configured to be coupled to an insertion tool such that distal movement of the retention member and the elongate member along a longitudinal axis of the elongate member relative to the insertion tool is limited.

As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the words “proximal” and “distal” refer to the direction closer to and away from, respectively, an operator (e.g., surgeon, physician, nurse, technician, etc.) who would insert the medical device into the patient, with the tip-end (i.e., distal end) of the device inserted inside a patient's body first. Thus, for example, the end of a medical device first inserted inside the patient's body would be the distal end, while the opposite end of the medical device (e.g., the end of the medical device being operated by the operator) would be the proximal end of the medical device.

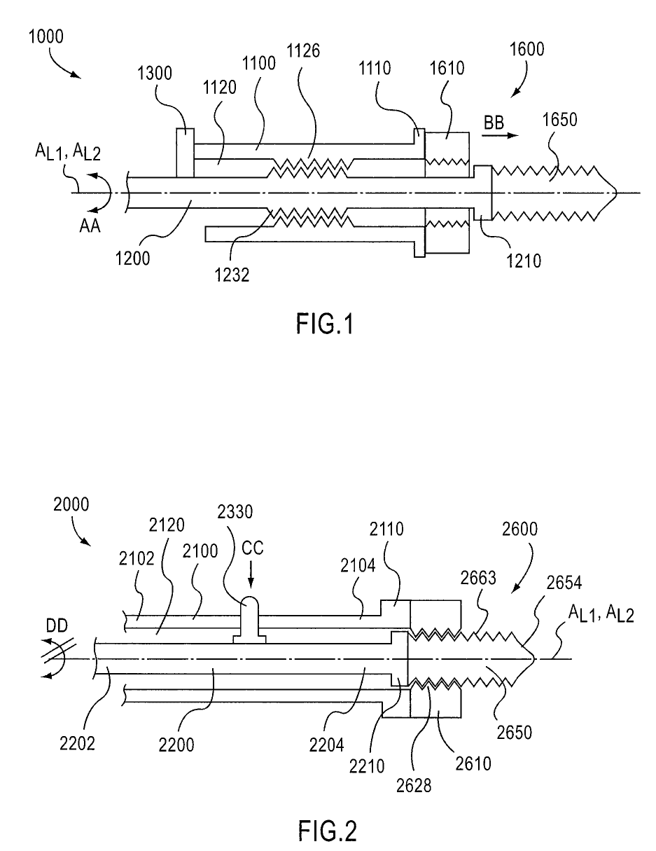

FIG. 1 is a schematic illustration of a medical device 1000 according to an embodiment of the invention. The medical device 1000 includes a first shaft 1100, a second shaft 1200, and a locking mechanism 1300. The first shaft 1100 includes an engagement portion 1110 and a threaded portion 1126. The first shaft 1100 defines a longitudinal axis A_{L1} and a lumen 1120 that is substantially concentric with the longitudinal axis A_{L1}. The threaded portion 1126 of the first shaft 1110 is disposed within the lumen 1120. Said another way, the threaded portion 1126 includes female threads within the lumen 1120. Although the threaded portion 1126 is shown as being disposed within the lumen 1120, in other embodiments, the threaded portion 1126 can be disposed in any suitable location of the first shaft 1100.

The engagement portion 1110 of the first shaft 1100 is configured to engage a first member 1610 of a bone fixation device 1600. The first member 1610 can be, for example, a nut configured to be threadedly coupled to a second member 1650 (e.g., a screw) of the bone fixation device 1600. As described in more detail herein, the engagement portion 1110 can include any suitable mechanism for engaging, retaining and/or being selectively coupled to the first member 1610 of the bone fixation device 1600. For example, in some embodiments, the first member 1610 of the bone fixation device 1600 can include a hexagonal shaped outer surface (not shown in FIG. 1) configured to be received within a corresponding recess (not shown in FIG. 1) defined by the engagement portion 1110 of the first shaft 1100.

The second shaft 1200 includes an engagement portion 1210 and a threaded portion 1232, and defines a longitudinal axis A_{L2}. The threaded portion 1232 of the second shaft 1210 defines a portion of an outer surface of the second shaft 1200. Said another way, the threaded portion 1232 of the second shaft 1200 includes male threads on a portion of the outer surface of the second shaft 1200. At least a portion of the second shaft 1200 is disposed within the lumen 1120 of the first shaft 1100 such that the longitudinal axis A_{L2} of the second shaft 1200 is substantially coincident with the longitudinal axis A_{L1} of the first shaft 1100. Moreover, the second shaft 1200 is disposed within the lumen 1120 of the first shaft 1100 such that the threaded portion 1126 of the first shaft 1100 is engaged with the threaded portion 1232 of the second shaft 1200. Said another way, the threaded portion 1126 of the first shaft 1100 corresponds to the threaded portion 1232 of the second shaft 1200 such that when a portion of the second shaft 1200 is disposed within the lumen 1120 of the first shaft 1100, the threaded portion 1232 of the second shaft 1200 can be matingly engaged with the threaded portion 1126 of the first shaft 1100. Said yet another way, the thread pitch of the threaded portion 1126 of the first shaft 1100 is substantially the same as the thread pitch of the threaded portion 1232 of the second shaft 1200 such that when a portion of the second shaft 1200 is disposed within the lumen 1120 of the first shaft 1100, the threaded portion 1232 of the second shaft 1200 can be threadedly coupled to the threaded portion 1126 of the first shaft 1100.

The engagement portion 1210 of the second shaft 1200 is configured to engage a second member 1650 of the bone fixation device 1600. The second member 1650 can be, for example, a bone screw configured to be threadedly coupled to the first member 1610 (e.g., a nut) of the bone fixation device 1600. As described in more detail herein, the engagement portion 1210 of the second shaft 1200 can include any suitable mechanism for engaging, retaining and/or being selectively coupled to the second member 1650 of the bone fixation device 1600. For example, in some embodiments, the second member 1650 of the bone fixation device 1600 can define a hexagonal shaped recess (not shown in FIG. 1) configured to receive a corresponding protrusion (not shown in FIG. 1) of the engagement portion 1210 of the second shaft 1200.

The locking mechanism 1300 is configured to selectively engage the first shaft 1100 and/or the second shaft 1200 to selectively allow the second shaft 1200 to rotate relative to the first shaft 1100, as indicated by the arrow AA in FIG. 1. Said another way, the locking mechanism 1300 is configured to selectively allow the second shaft 1200 to rotate within the first shaft 1100 about the longitudinal axis A_{L2}. Said yet another way, the locking mechanism 1300 is configured to selectively allow the first shaft 1100 to rotate about the second shaft 1200 about the longitudinal axis A_{L1}. In some embodiments, as described in more detail herein, the locking mechanism 1300 can be moved between a first configuration and a second configuration. When the locking mechanism 1300 is in the first configuration, the locking mechanism 1300 is configured to prevent and/or limit the rotation of the second shaft 1200 with respect to the first shaft 1100. When the locking mechanism 1300 is in the second configuration, the locking mechanism 1300 is configured to allow the rotation of the second shaft 1200 with respect to the first shaft 1100.

The locking mechanism 1300 can be any mechanism suitable for selectively allowing the second shaft 1200 to rotate relative to the first shaft 1100. For example, in some embodiments, the locking mechanism 1300 can include a ratcheting and/or a clutching mechanism configured to allow the second shaft 1200 to selectively rotate unidirectionally within the first shaft 1100. In other embodiments, the locking mechanism 1300 can include a ratcheting and/or a clutching mechanism configured to allow the second shaft 1200 to rotate in discrete increments relative to the first shaft 1100. In this manner, the locking mechanism can allow a user to rotate the second shaft 1200 relative to the first shaft 1100 in a controlled and/or incremental fashion.

As described in more detail herein, the medical device 1000 can be used insert, position and/or install the bone fixation device 1600 within a targeted bone tissue. In some embodiments, for example, the medical device 1000 can be used to rotatably insert the bone fixation device 1600 into a portion of a spine as a part of a spinal fixation procedure. In use, the bone fixation device 1600 can be coupled to the medical device 1000 prior to inserting the bone fixation device 1600 into the body. Similarly stated, the first member 1610 of the bone fixation device 1600 can be coupled to the engagement portion 1110 of the first shaft 1100 and the second member 1650 of the bone fixation device 1600 can be coupled to the engagement portion 1210 of the second shaft 1200 prior to insertion into the body. Although the first member 1610 is shown as being spaced apart from the second member 1650, in some embodiments, the first member 1610 can be coupled to, engaged with, and/or disposed about the second member 1650 when the bone fixation device 1600 is coupled to the medical device 1000 and prior to insertion into the body.

The bone fixation device 1600 and a distal portion of the medical device 1000 can then be inserted into the body and disposed adjacent the targeted bone tissue. In some embodiments, for example, the bone fixation device 1600 can be inserted percutaneously and/or in a minimally-invasive manner. The second member 1650 of the bone fixation device 1600 can be inserted into the targeted bone tissue by rotating the second shaft 1200, as indicated by the arrow AA in FIG. 1. In this manner, the second member 1650 can be threaded into and/or rotatably disposed within the targeted bone tissue. In some embodiments, the first shaft 1100 can be maintained in a constant rotational position while the second shaft 1200 is rotated. Said another way, the locking mechanism 1300 can be in an unlocked configuration thereby allowing the second shaft 1200 to be rotated within the first shaft 1100. When the second shaft 1200 is rotated within the first shaft 1100, the threaded portion 1232 of the second shaft 1200 moves relative to the threaded portion 1126 of the first shaft, resulting in axial movement of the second shaft 1200 relative to the first shaft 1100 in a distal direction, as indicated by the arrow BB in FIG. 1. Accordingly, when the second shaft 1200 is rotated within the first shaft 1100, second member 1650 of the bone fixation device 1600 is moved axially relative to the first member 1610 of the bone fixation device.

In other embodiments, however, the second shaft 1200 and the first shaft 1100 can be rotated together to install the second member 1650 of the bone fixation device 1600 into the targeted bone tissue. Said another way, the locking mechanism 1300 can be in a locked configuration thereby preventing the second shaft 1200 from rotating relative to the first shaft 1100. Accordingly, the second shaft 1200 does not move axially relative to the first shaft 1100, and the second member 1650 of the bone fixation device 1600 does not move axially relative to the first member 1610 of the bone fixation device 1600.

The first member 1610 of the bone fixation device 1600 can then be moved into engagement with the targeted bone tissue by rotating the first shaft 1100 while maintaining the second shaft 1200 in a constant rotational position. Said another way, the locking mechanism 1300 can be in the unlocked configuration, thereby allowing the first shaft 1100 to rotate about the second shaft 1200. Accordingly, the first member 1610 of the bone fixation device 1600 is rotated relative to the second member 1650 of the bone fixation device 1600. Moreover, as described above, the threaded portion 1232 of the second shaft 1200 moves relative to the threaded portion 1126 of the first shaft, resulting in axial movement of the second shaft 1200 relative to the first shaft 1100 in a distal direction and by a predetermined distance (associated with the pitch of the threaded portion 1126 and the threaded portion 1232). In this manner, the first member 1610 of the bone fixation device can be moved axially relative to the second member 1650 of the bone fixation device 1600 by the predetermined distance. In some embodiments, for example, the first member 1610 of the bone fixation device 1600 can be threaded onto the second member 1650 of the bone fixation device 1600. In this manner, the first member 1610 of the bone fixation device 1600 can be moved axially relative to the second member 1650 of the bone fixation device 1600 until the first member 1610 is in contact with the targeted tissue and/or a predetermined clamping load is attained.

After the bone fixation device 1600 is installed within the targeted bone tissue, the engagement portion 1110 of the first shaft 1100 can be decoupled from the first member 1610, and the engagement portion 1210 of the second shaft 1200 can be decoupled from the second member 1650. The medical device 1000 can then be removed from the body.

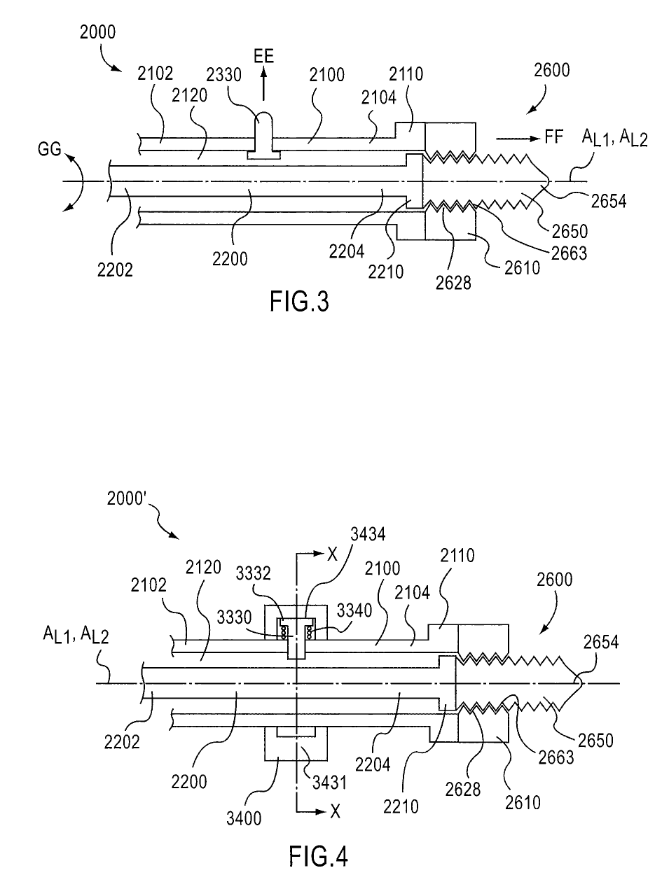

Although the first shaft 1100 and the second shaft 1200 are shown and described above as being threadedly engaged, in other embodiments, a medical device can include a first shaft and a second shaft devoid of a threaded engagement. For example, FIGS. 2 and 3 are schematic illustrations of a medical device 2000 according to an embodiment of the invention. As described in more detail herein, the medical device 2000 can be used to insert a bone fixation device 2600 into a targeted bone tissue (not shown in FIGS. 2 and 3). The bone fixation device 2600 includes a bone screw 2650 and a nut 2610. The bone screw 2650 includes a threaded portion 2663 and a self-tapping distal tip 2654. Accordingly, the bone screw 2650 can be rotatably disposed within the targeted bone tissue. Similarly, the nut 2610 includes a threaded portion 2628. Accordingly, the nut 2610 can be threadedly coupled to the bone screw 2650 such that the nut 2610 can engage a surface of the targeted bone tissue to apply a clamping load.

The medical device 2000 includes a first shaft 2100, a second shaft 2200, and a lock tab 2330. The first shaft 2100 has a proximal end portion 2102 and a distal end portion 2104. The first shaft 2100 defines a longitudinal axis A_{L1} and a lumen 2120 that is substantially concentric with the longitudinal axis A_{L1}. The distal end portion 2104 includes an engagement portion 2110. The engagement portion 2110 of the first shaft 2100 is configured to engage the nut 2610 of the bone fixation device 2600. As described in more detail herein, the engagement portion 2110 can include any suitable mechanism for engaging, retaining and/or being selectively coupled to the nut 2610 of the bone fixation device 2600. For example, in some embodiments, the nut 2610 of the bone fixation device 2600 can include a hexagonal shaped outer surface (not shown in FIGS. 2 and 3) configured to be received within a corresponding recess (not shown in FIGS. 2 and 3) defined by the engagement portion 2110 of the first shaft 2100.

The second shaft 2200 includes a proximal end portion 2202 and a distal end portion 2204. The distal end portion 2204 of the second shaft 2200 includes an engagement portion 2210. The engagement portion 2210 of the second shaft 2200 is configured to engage the bone screw 2650 of the bone fixation device 2600 when the nut 2610 is threadedly coupled to the bone screw 2650. The engagement portion 2210 of the second shaft 2200 can include any suitable mechanism for engaging, retaining and/or being selectively coupled to the bone screw 2650 of the bone fixation device 2600. For example, in some embodiments, the bone screw 2650 can define a hexagonal shaped recess (not shown in FIGS. 2 and 3) configured to receive a corresponding protrusion (not shown in FIGS. 2 and 3) of the engagement portion 2210 of the second shaft 2200.

At least a portion of the second shaft 2200 is disposed within the lumen 2120 of the first shaft 2100 such that a longitudinal axis A_{L2} of the second shaft 2200 is substantially coincident with the longitudinal axis A_{L1} of the first shaft 2100. Moreover, as described in more detail below, the second shaft 2200 is coupled to the first shaft 2100 such that when the first shaft 2100 rotates about the second shaft 2200, the first shaft 2100 is configured to move an axial distance relative to the second shaft 2200. Said another way, the second shaft 2200 is coupled to the first shaft 2100 such that rotation of the second shaft 2200 within the first shaft 2100 results in axial movement of the second shaft 2200 relative to the first shaft 2100. In this manner, in some embodiments, when the first shaft 2100 rotates about the second shaft 2200, the axial position of the engagement portion 2110 of the first shaft 2100 relative to the engagement portion 2210 of the second shaft 2200 can be adjusted by a predetermined amount (i.e., based on the amount of rotation of the first shaft 2100 relative to the second shaft 2200). In some embodiments, for example, the axial position of the engagement portion 2110 of the first shaft 2100 relative to the engagement portion 2210 of the second shaft 2200 can be adjusted to match the axial position of the nut 2610 relative to the bone screw 2650.

The second shaft 2200 can be coupled to the first shaft 2100 in any suitable manner. For example, in some embodiments, the second shaft 2200 can include a protrusion (not shown in FIGS. 2 and 3) that is disposed within a spiral groove (not shown in FIGS. 2 and 3) defined by the first shaft 2100. In this manner, when the first shaft 2100 rotates about the second shaft 2200, the protrusion will travel within the spiral groove thereby causing the first shaft 2100 to move axially relative to the second shaft 2200.

The lock tab 2330 is configured to selectively engage the first shaft 2100 and/or the second shaft 2200 to selectively allow the second shaft 2200 to rotate relative to the first shaft 2100. As shown by the arrow CC in FIG. 2 and the arrow EE in FIG. 3, the lock tab 2330 can be moved between a first configuration (FIG. 2) and a second configuration (FIG. 3). When the lock tab 2330 is in the first configuration, the lock tab 2330 is configured to engage a portion of the second shaft 2200 to prevent and/or limit the rotation of the second shaft 2200 with respect to the first shaft 2100, as shown by the arrow DD in FIG. 2. When the lock tab 2330 is in the second configuration, the lock tab 2330 is spaced apart from the second shaft 2200 to allow the rotation of the second shaft 2200 with respect to the first shaft 2100, as indicated by the arrow GG in FIG. 3. As described above, when the second shaft 2200 rotates within the first shaft 2100, the first shaft 2100 is configured to move a predetermined axial distance relative to the second shaft 2200, as indicated by the arrow FF in FIG. 3.

In some embodiments, the medical device 2000 can include an actuator configured to move the lock tab 2300 between the first configuration and the second configuration. For example, FIGS. 4 and 5 show a medical device 2000′ including an actuator 3400 and a biased locking mechanism 3300. The locking mechanism 3300 includes a lock tab 3330 and a biasing member 3340. The lock tab 3330, which is similar to the lock tab 2330 shown and described above with reference to FIGS. 2 and 3, includes a flange 3332. The biasing member 3340, which can be, for example, a spring, a Bellville washer or the like, is disposed between the outer surface of the first shaft 2100 and the flange 3332 of the lock tab 3330. In this manner, the biasing member 3340 can bias the lock tab 3330 in the second (or unlocked) configuration. In other embodiments, however, the biasing member 3340 can be configured to bias the lock tab 3330 in the first (or locked) configuration. As described above, when the lock tab 3330 is in the first configuration (not shown in FIGS. 3 and 4), the lock tab 3330 is configured to engage a portion of the second shaft 2200 to prevent and/or limit the rotation of the second shaft 2200 with respect to the first shaft 2100. When the lock tab 3330 is in the second configuration, the lock tab 3330 is spaced apart from the second shaft 2200 to allow the rotation of the second shaft 2200 with respect to the first shaft 2100.

The actuator 3400 includes a side wall 3431 that defines a recess 3432. The side wall 3431 of the actuator 3400 includes a cam surface 3434 and an end surface 3433. The cam surface 3434 is a curved surface having a radius of curvature that is offset from the longitudinal axis A_{L1} of the first shaft 2100. In this manner, as shown in FIG. 5, the distance between the cam surface 3434 and the first shaft 2100 varies circumferentially. The lock tab 3330 is disposed within the recess 3432 such that the flange 3332 of the lock tab 3330 is in contact with the cam surface 3434.

The actuator 3400 is rotatably coupled to the first shaft 2100. Said another way, the actuator 3400 is coupled to the first shaft 2100 such that the actuator 3400 can rotate relative to the first shaft 2100 about the longitudinal axis A_{L1}. Accordingly, when the actuator 3400 is rotated about the first shaft 2100, as shown by the arrow HH in FIG. 5, the cam surface 3434 can move the lock tab 3330 between the first configuration and the second configuration (shown in FIGS. 4 and 5). Moreover, the end surface 3433 of the actuator 3400 can engage at least a portion of the lock tab 3330 to maintain the rotational position of the actuator 3400 relative to the first shaft 2100. Said another way, the end surface 3433 of the actuator 3400 can limit the rotation of the actuator 3400 about the first shaft 2100.

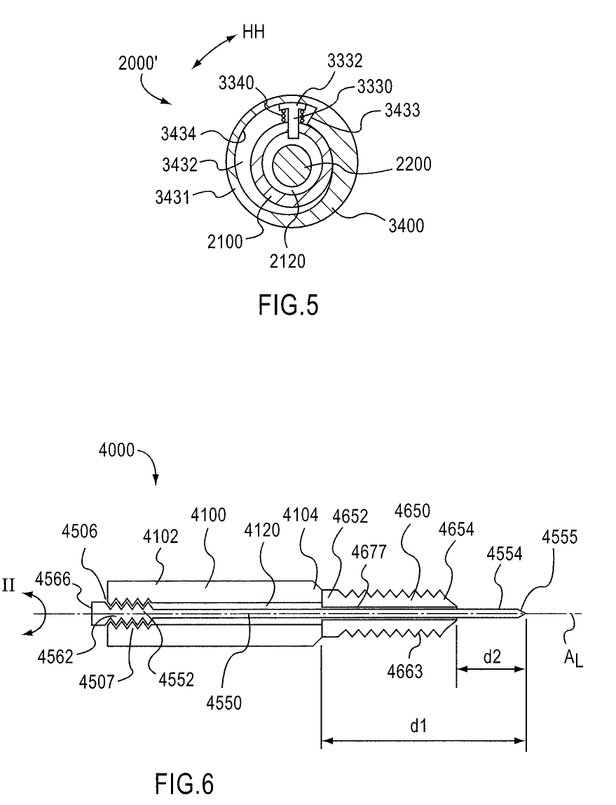

Although the second shaft 2200 of the medical device 2000 is shown as including an engagement portion 2210 that engages a proximal end of the bone screw 2650, in other embodiments, an insertion tool can include a second shaft that is disposed within a cannulated bone screw. For example, FIG. 6 is a schematic illustration of a medical device 4000 according to an embodiment of the invention. The medical device 4000 can be used to insert a bone fixation device 4650 into a targeted bone tissue (not shown in FIG. 6). The bone screw 4650 includes a proximal end 4652, a self-tapping distal tip 4654, and a threaded portion 4663. The bone screw 4650 defines a lumen 4677 therethrough (i.e., the bone screw 4650 is a cannulated bone screw).

The medical device 4000 includes an outer shaft 4100 and an inner shaft 4550. The outer shaft 4100 includes a proximal end portion 4102 and a distal end portion 4104. The outer shaft 4100 defines a longitudinal axis A_{L} and a lumen 4120 that is substantially concentric with the longitudinal axis A_{L}. The distal end portion 4104 is configured to engage and/or retain the bone screw 4650. The distal end portion 4104 can include any suitable mechanism for engaging, retaining and/or being selectively coupled to the bone screw 4650. For example, in some embodiments, distal end portion 4104 of the outer shaft 4100 can retain the bone screw 4650 by a mechanical coupling (e.g., mating features, a snap ring arrangement, or the like), a magnetic coupling, and/or a chemical couple (e.g., adhesive).

The proximal end portion 4102 of the outer shaft 4100 defines an opening 4506 in fluid communication with the lumen 4120. The proximal end portion of the lumen includes a threaded portion 4507. Said another way, the proximal end portion 4102 of the outer shaft 4100 defines an opening 4506 having female threads 4507.

The inner shaft 4550, which can be, for example, a guide wire, a Kirschner wire (e.g., a K-wire) or the like, includes a proximal end portion 4552 and a distal end portion 4554. The distal end portion 4554 of the inner shaft 4550 includes a tapered tip 4555 configured to pierce, dilate and or distract bodily tissue. In some embodiments, for example, the tapered tip 4555 can be configured to pierce bone tissue. The proximal end portion 4552 of the inner shaft 4550 includes a threaded portion 4562 and a proximal end surface 4566. The threaded portion 4562 of the inner shaft 4550 is disposed on an outer surface of the inner shaft 4550. Said another way, the threaded portion 4562 of the inner shaft 4550 includes male threads on the outer surface of the inner shaft 4550.

At least a portion of the inner shaft 4550 is disposed within the lumen 4120 of the outer shaft 4100 such that the threaded portion 4507 of the outer shaft 4100 is engaged with the threaded portion 4562 of the inner shaft 4550. Said another way, the threaded portion 4507 of the outer shaft 4100 corresponds to the threaded portion 4562 of the inner shaft 4550 such that when a portion of the inner shaft 4550 is disposed within the lumen 4120 of the outer shaft 4100, the threaded portion 4562 of the inner shaft 4550 can be matingly engaged with the threaded portion 4126 of the outer shaft 4100. Said yet another way, when a portion of the inner shaft 4550 is disposed within the lumen 4120 of the outer shaft 4100, the threaded portion 4562 of the inner shaft 4550 can be threadedly coupled to the threaded portion 4126 of the outer shaft 4100.

Moreover, when the portion of the inner shaft 4550 is disposed within the lumen 4120 of the outer shaft 4100, the tapered tip 4555 of the inner shaft 4550 is spaced apart from the distal end portion 4104 of the outer shaft 4100 by a distance d1. In this manner, when the portion of the inner shaft 4550 is disposed within the lumen 4120 of the outer shaft 4100, the distal end portion 4554 of the inner shaft 4550 is disposed partially within the lumen 4677 of the bone screw 4650 such that the tapered tip 4555 of the inner shaft 4550 is spaced apart from the distal tip 4654 of the bone screw 4650 by a distance d2. As described in more detail below, the distance d1 and/or the distance d2 can be adjusted by rotating the inner shaft 4550 relative to the outer shaft 4100, as indicated by the arrow II in FIG. 6. Accordingly, in some embodiments, the tapered tip 4555 of the inner shaft 4550 can spaced apart from the distal tip 4654 of the bone screw 4650 and/or the distal end portion 4104 of the outer shaft 4100 by a predetermined distance.

The medical device 4000 can be used insert, position and/or install the bone screw 4650 within a targeted bone tissue (not shown in FIG. 6). In some embodiments, for example, the medical device 4000 can be used to rotatably insert the bone screw 4650 into a portion of a spine as a part of a spinal fixation procedure. In use, the bone screw 4650 can be removably coupled to the distal end portion 4104 of the outer shaft 4100. The inner shaft 4550 can be disposed partially within the lumen 4120 of the outer shaft 4100 such that the tapered tip 4555 of the inner shaft 4550 is spaced apart from the distal tip 4654 of the bone screw 4650.

The bone screw 4650, the distal end portion 4104 of the outer shaft 4100, and the distal end portion 4554 of the inner shaft 4550 can be collectively inserted into the body and disposed adjacent the targeted bone tissue. During the insertion processes, the tapered tip 4555 can be used to pierce and/or dilate bodily tissue. Moreover, after the distal end portion 4554 of the inner shaft 4550 is disposed against the targeted bone tissue (i.e., “docked” against the targeted bone tissue), the tapered tip 4555 can be advanced into the targeted bone tissue. Said another way, the tapered tip 4555 can be used to define a passageway within the targeted bone tissue within which the bone screw 4650 can be disposed. In some embodiments, a user can impart a force (e.g., via a hammer) on the proximal end surface 4566 of the inner shaft 4550 to advance the tapered tip 4555 into the targeted bone tissue.

After the distal end portion 4554 of the inner shaft 4550 is disposed within the targeted bone tissue, the bone screw 4550 can be inserted into the targeted bone tissue by rotating the outer shaft 4100 about the longitudinal axis A_{L}. In some embodiments, the inner shaft 4550 can then be rotated relative to the outer shaft 4100 to adjust the axial distance between the tapered tip 4555 of the inner shaft 4550 and the distal tip 4654 of the bone screw 4650. For example, in some embodiments, after the bone screw 4650 is partially inserted into the targeted bone tissue, the inner shaft 4550 can be rotated relative to the outer shaft 4100 to adjust the axial distance between the tapered tip 4555 of the inner shaft 4550 and the distal tip 4654. In this manner, the tapered tip 4555 can be advanced further into the targeted bone tissue, thereby extending the passageway within the targeted bone tissue.

After the bone screw 4650 is installed within the targeted bone tissue, the outer shaft 4100 can be decoupled from the bone screw 4650 and the medical device 4000 can be removed from the body. In some embodiments, the inner shaft 4550 can be removed from the body before the outer shaft 4100 is decoupled from the bone screw 4650.

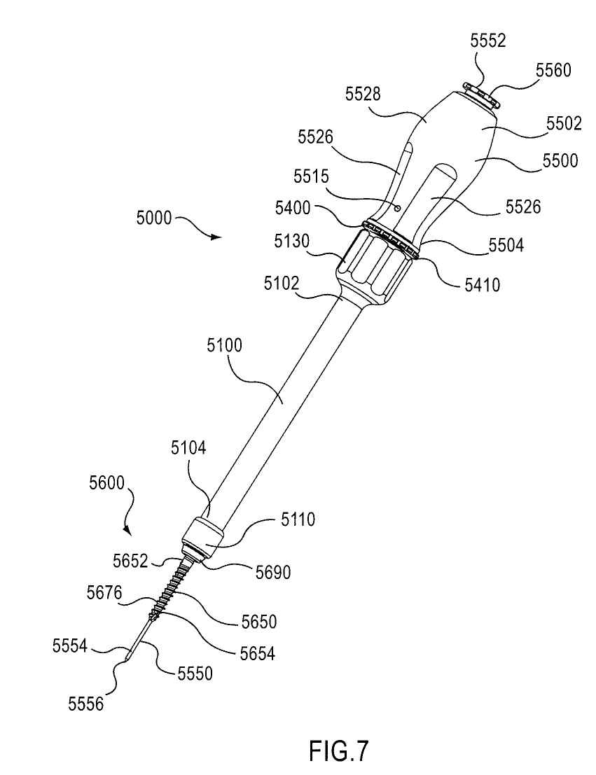

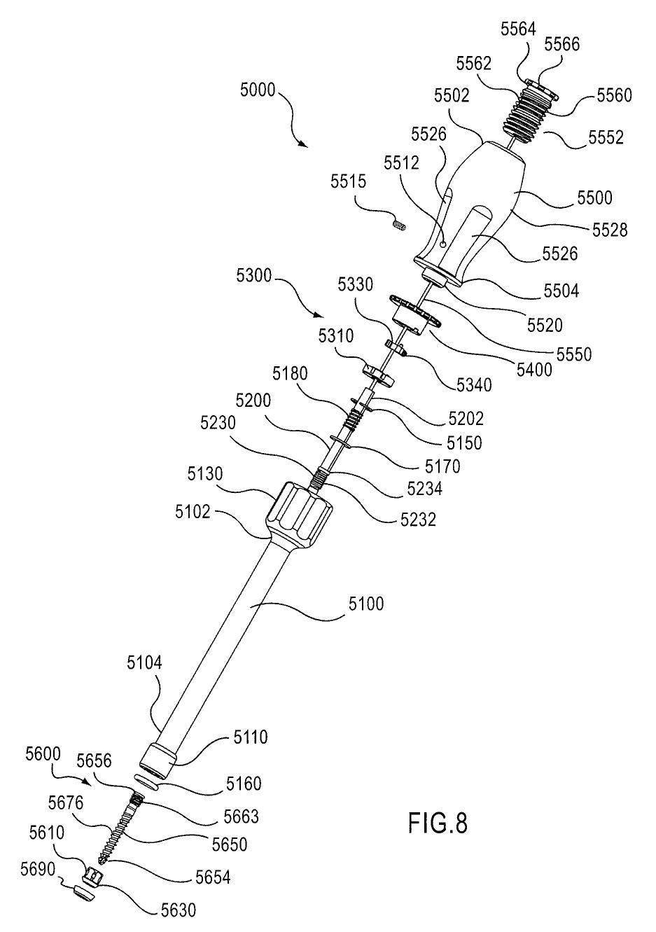

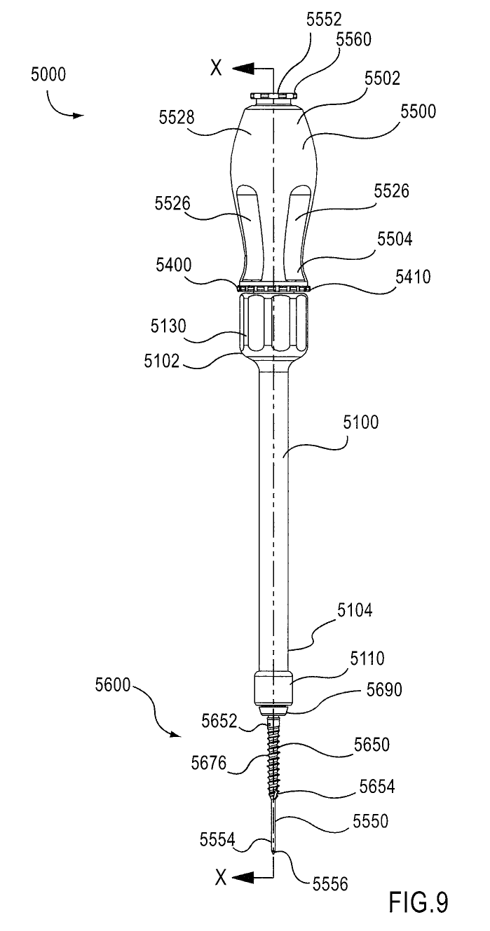

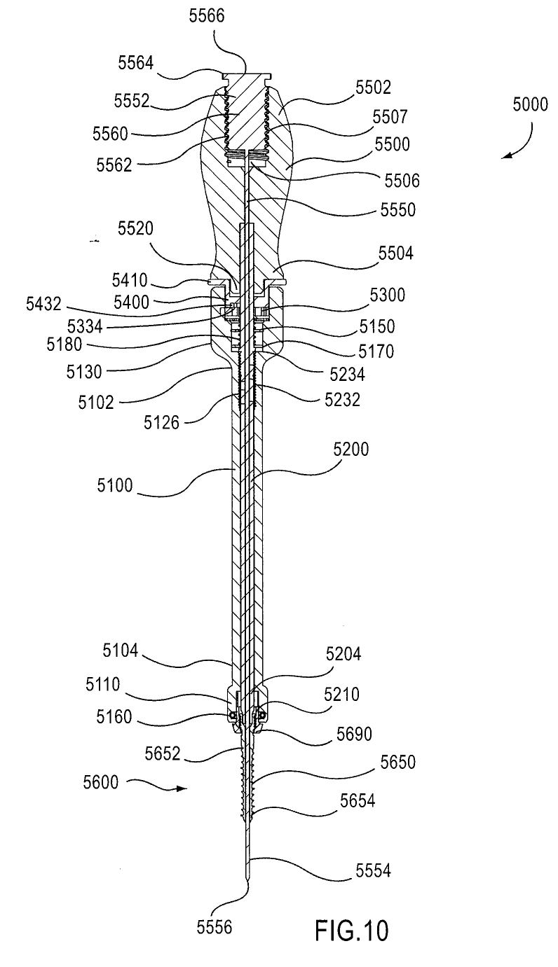

FIGS. 7-10 show an insertion tool 5000 and a bone fixation device 5600 according to an embodiment of the invention. More particularly, FIG. 7 is a perspective view of the insertion tool 5000 coupled to the bone fixation device 5600. FIG. 8 is an exploded view of the insertion tool 5000 and the bone fixation device 5600. FIGS. 9 and 10 are a front view and a cross-sectional view, respectively, of the insertion tool 5000 and the bone fixation device 5600. The insertion tool 5000 includes a first shaft 5100, a second shaft 5200 (see FIG. 8), a locking mechanism 5300 (see FIG. 8), an actuator 5400, a handle 5500 and a guide wire 5550. The bone fixation device 5600 includes a nut 5610 and a bone screw 5650. A detailed description of each of the components contained in the insertion tool 5000 and the bone fixation device 5600 is presented below, followed by a step-by-step description of operation of the insertion tool 5000.

The first shaft 5100, which can also be referred to as the outer shaft or the nut driver shaft, includes a proximal end portion 5102 and a distal end portion 5104. The first shaft 5100 defines a lumen 5120 therethrough. As shown in FIGS. 14 and 16, the lumen 5120 defines a longitudinal axis A_{L1}, and includes a proximal portion 5122, a distal portion 5124 and a threaded portion 5126. Similarly stated, the threaded portion 5126 includes female threads within the lumen 5120. Although the threaded portion 5126 is shown as being disposed within proximal portion 5122 of the lumen 5120, in other embodiments, the threaded portion 5126 can be disposed in any suitable location within the lumen 5126.

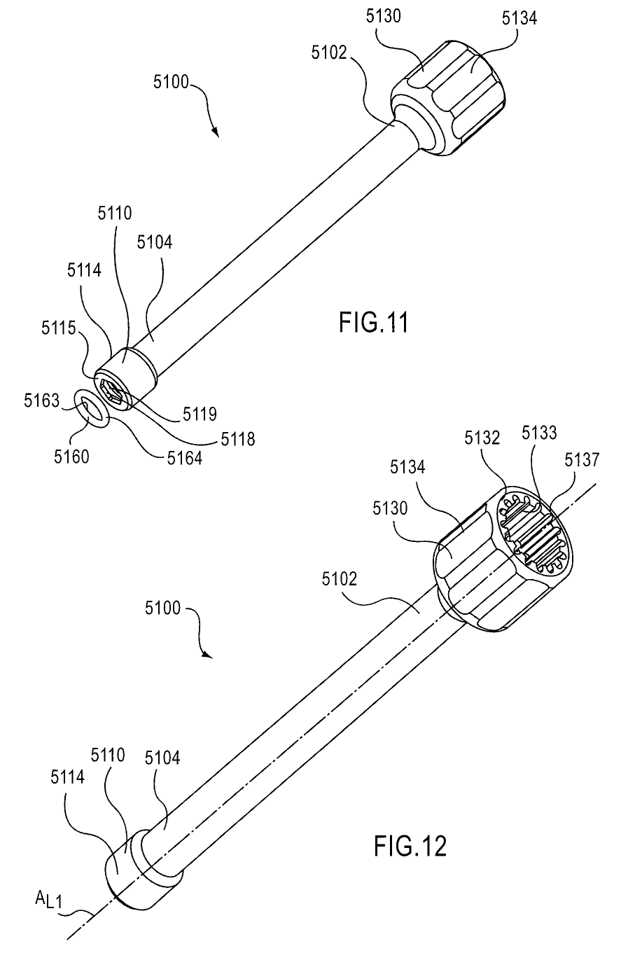

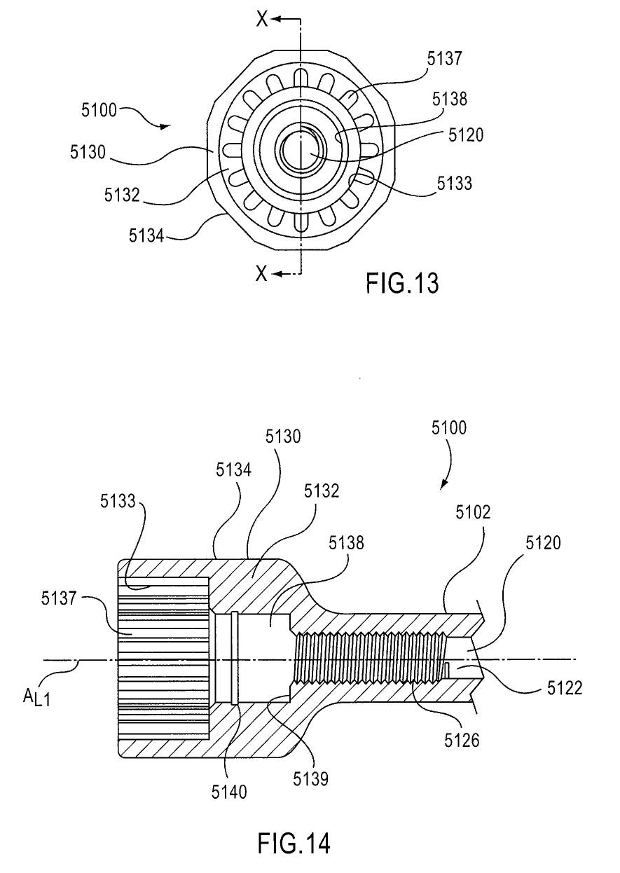

As shown in FIGS. 11-14, the proximal end portion 5102 of the first shaft 5100 includes an actuation portion 5130. The actuation portion 5130 includes a side wall 5132 having an outer surface 5134 and an inner surface 5133. As shown in FIG. 12, the outer surface 5134 includes multiple alternating protrusions and recesses along the longitudinal axis A_{L1} of the first shaft 5100. In this manner, the outer surface 5134 of the actuation portion 5130 is configured to be grasped and/or manipulated by the user, for example, to rotate the first shaft 5100 about the second shaft 5200. Although the outer surface 5134 is shown as including multiple alternating protrusions and recesses, in other embodiments, the outer surface 5134 can include any suitable topographical features to aid in the manipulation of the first shaft 5100. For example, in some embodiments, the outer surface 5134 can be knurled, cross-hatched or the like.

The inner surface 5133 of the actuation portion 5130 defines series of splines 5137 and a spring pocket 5138. The splines 5137 are substantially parallel to the longitudinal axis A_{L1} of the first shaft 5100. Said another way, a portion of the inner surface 5133 of the actuation portion 5130 defines multiple alternating protrusions and grooves along the longitudinal axis A_{L1}.

The spring pocket 5138 is disposed distally from the splines 5137 (see FIG. 14) and is in fluid communication with the proximal portion 5122 of the lumen 5120. As shown in FIGS. 8 and 10, the spring pocket 5138 is configured to receive a portion of the second shaft 5200 and the spring 5180. The spring pocket 5138 includes a shoulder 5139 (see FIG. 14) configured to be engaged with a corresponding shoulder 5234 of the second shaft 5200. The portion of the inner surface 5133 defining the spring pocket 5138 also defines a circumferential groove 5140. As described in more detail herein the circumferential groove 5140 is configured to receive a retaining ring 5150 (e.g., a snap ring).

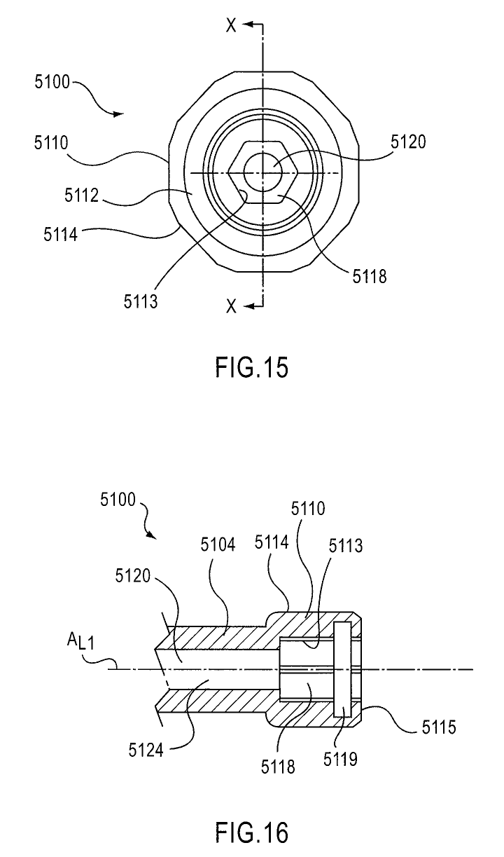

As shown in FIGS. 11, 12, 15 and 16, the distal end portion 5104 of the first shaft 5100 includes a nut engagement portion 5110. The nut engagement portion 5110 includes a side wall 5112 having an outer surface 5114, an inner surface 5113, and a distal end surface 5115. The inner surface 5113 of the nut engagement portion 5110 defines an opening 5118 configured to receive the nut 5610 of the bone fixation device 5600. The opening 5118 is in fluid communication with the distal portion 5124 of the lumen 5120. The inner surface 5113 includes a set of hexagonal shaped portions corresponding to the hexagonal flats 5621 (see e.g., FIG. 43) of the nut 5610. Moreover, the inner surface 5113 of the nut engagement portion 5110 defines a groove 5119 that receives a nut retention member 5160. The nut retention member 5160 can be, for example, a snap ring configured to maintain a position of the nut 5610 relative to the first shaft 5100. In this manner, the nut engagement portion 5110 of the first shaft 5100 can selectively retain the nut 5610 to limit movement of the nut 5610 relative to the first shaft 5100 along the longitudinal axis A_{L1}.

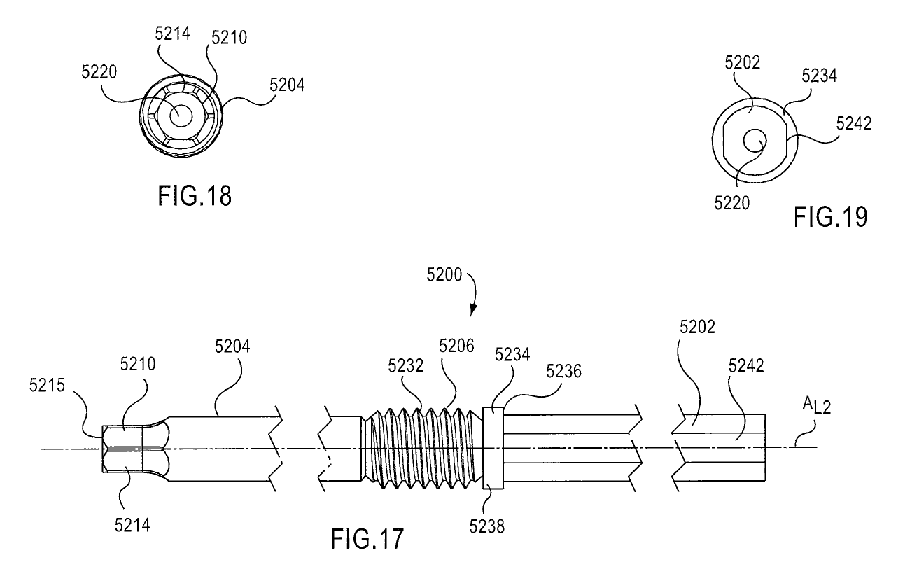

As best shown in FIG. 17, the second shaft 5200, which can also be referred to as the inner shaft or the hex driver shaft, includes a proximal end portion 5202, a distal end portion 5204, and a central portion 5206 disposed therebetween. The second shaft 5200 defines a lumen 5220 (see FIGS. 18 and 19) that defines a longitudinal axis A_{L2}. As shown in FIG. 8, the proximal end portion 5202 of the second shaft 5200 is configured to be received within the handle 5500. More particularly, as shown in FIGS. 17 and 19, the proximal end portion 5202 of the second shaft 5200 includes two flatted surfaces 5242 that correspond to flatted surfaces within the distal opening 5509 of the handle 5500 such that when the second shaft 5200 is disposed within the handle 5500, the second shaft 5200 will rotate with the rotation of the handle 5500.

As shown in FIGS. 10, 17 and 18, the distal end portion 5204 of the second shaft 5200 includes an screw engagement portion 5210. The screw engagement portion 5210 includes an outer surface 5214 and a distal end surface 5215. The outer surface 5214 of the screw engagement portion 5210 includes a set of hexagonal shaped portions corresponding to the hexagonal shaped recess 5660 defined within the engagement portion 5656 of the bone screw 5650. In this manner, the screw engagement portion 5210 of the second shaft 5200 can be received within the engagement portion 5656 of the bone screw 5650 such that rotation of the second shaft 5200 about the longitudinal axis A_{L2} results in rotation of the bone screw 5650.

The central portion 5206 of the second shaft 5200 includes a threaded portion 5232 and a shoulder 5234. As shown in FIG. 17, the shoulder 5234 of the second shaft 5200 is disposed proximally from the threaded portion 5232 and includes a first surface 5236 and a second surface 5238. The threaded portion 5232 of the second shaft 5200 defines a portion of an outer surface of the second shaft 5200. Said another way, the threaded portion 5232 of the second shaft 5200 includes male threads on a portion of the outer surface of the second shaft 5200. The threaded portion 5232 of the second shaft 5200 corresponds to the threaded portion 5126 of the first shaft 5100. Said another way, the thread pitch of the threaded portion 5232 of the second shaft 5200 is substantially the same as the thread pitch of the threaded portion 5126 of the first shaft 5100. In this manner, as described in more detail herein, when the second shaft 5200 is disposed within the first shaft 5100, the threaded portion 5126 of the first shaft 5100 can be engaged with the threaded portion 5232 of the second shaft 5200. Although the threaded portion 5232 is shown as being disposed on the central portion 5206 of the second shaft 5200, in other embodiments, the threaded portion 5232 can be disposed in any suitable location along the second shaft 5200.

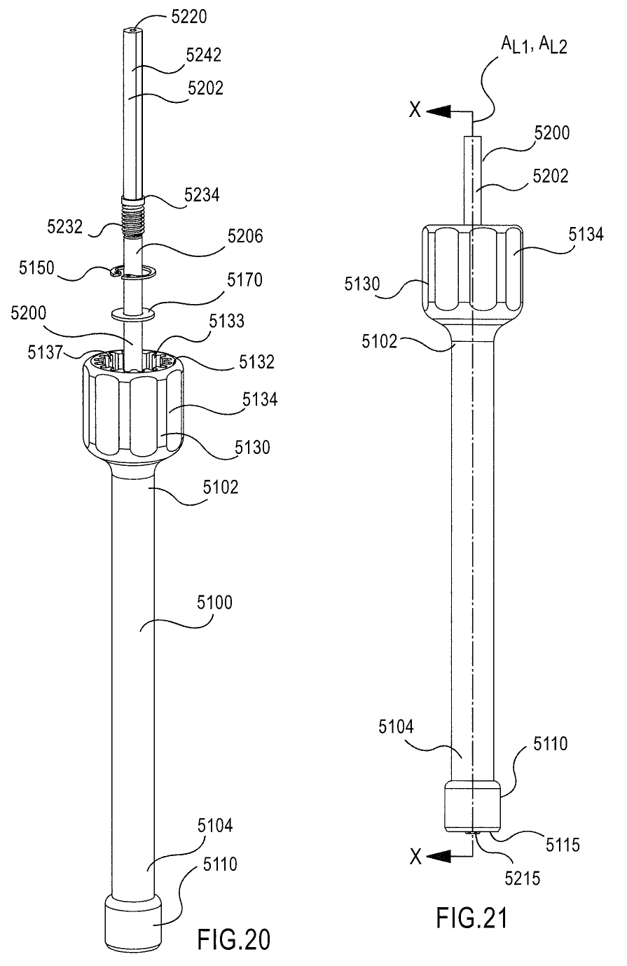

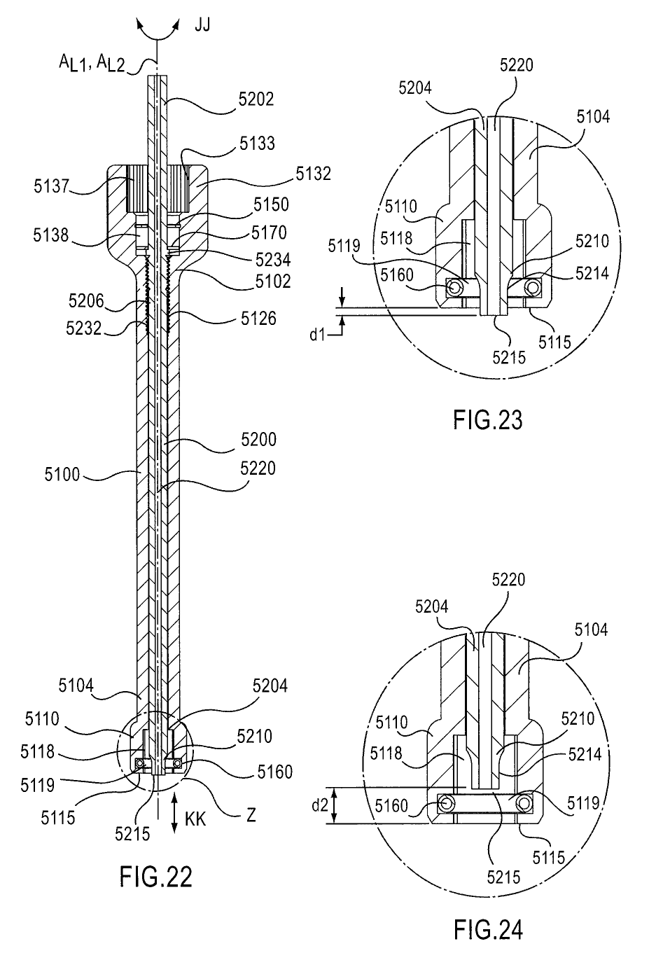

As shown in FIGS. 20-22, at least a portion of the second shaft 5200 is disposed within the lumen 5120 of the first shaft 5100 such that the longitudinal axis A_{L2} of the second shaft 5200 is substantially coincident with the longitudinal axis A_{L1} of the first shaft 5100. Moreover, the second shaft 5200 is disposed within the lumen 5120 of the first shaft 5100 such that the threaded portion 5126 of the first shaft 5100 is engaged with the threaded portion 5232 of the second shaft 5200. In this manner, when the second shaft 5200 rotates within the first shaft 5100, as indicated by the arrow JJ in FIG. 22, the second shaft 5200 moves axially relative to the first shaft 5100, as indicated by the arrow KK in FIG. 22. Similarly stated, when the first shaft 5100 rotates about the second shaft 5200, the first shaft 5100 moves axially relative to the second shaft 5200. The amount of axial movement of the first shaft 5100 relative to the second shaft 5200 is associated with the thread pitch of the threaded portion 5126 first shaft 5100 and/or the threaded portion 5232 of the second shaft 5200. In this manner, the first shaft 5100 can be moved axially relative to the second shaft 5200 in a controlled and/or incremental fashion.

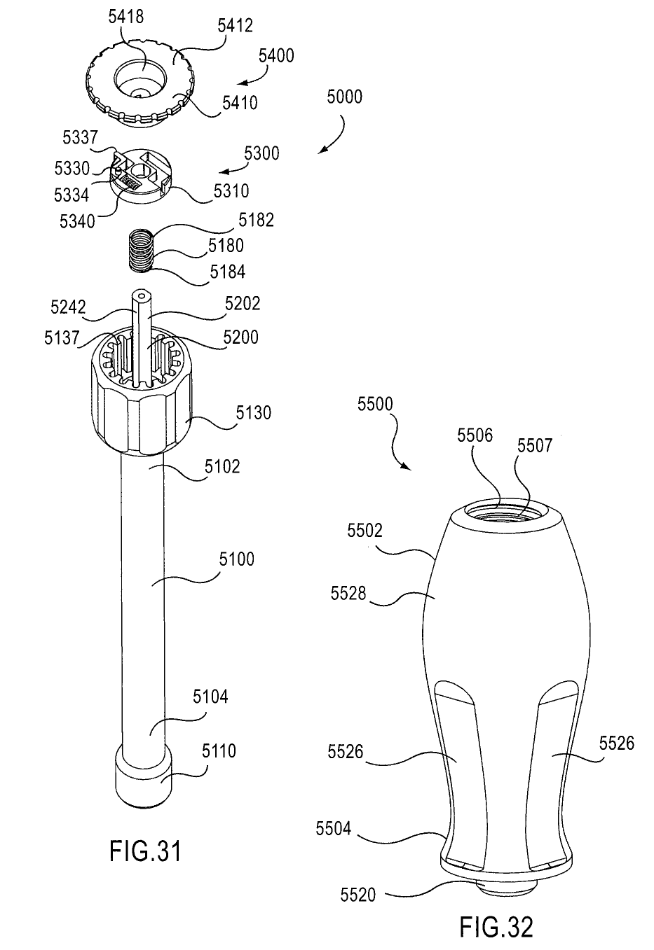

When the second shaft 5200 is disposed within the lumen 5120 of the first shaft 5100, a flat washer 5170 is disposed about the central portion 5206 of the second shaft 5200 and within the spring pocket 5138 of the first shaft 5100. The flat washer 5170 is disposed against the first surface 5236 of the shoulder 5234. In this manner, the flat washer 5170 is prevented from moving relative to the second shaft 5200 axially in a distal direction. As shown in FIGS. 10 and 31, a distal end 5184 of the spring 5180 is disposed against the second surface 5236 of the flat washer 5170 (note that the spring 5180 is not shown in FIGS. 20-22).

When the second shaft 5200 is disposed within the lumen 5120 of the first shaft 5100, the retaining ring 5150 is disposed within the circumferential groove 5140 of the first shaft 5100. In this manner, the retaining ring 5150 is maintained in a fixed longitudinal position within the spring pocket 5138. The retaining ring 5150 is spaced apart from the central portion of the second shaft 5200 such that the second shaft 5200 can move axially relative to the first shaft 5100 through a predetermined range of motion. When the second shaft 5200 is moved proximally relative to the first shaft 5100 through a predetermined distance, however, the retaining ring 5150 is configured to engage the second surface 5236 of the flat washer 5170. In this manner, the retaining ring 5150 can limit the axial movement of the second shaft 5200 within the first shaft 5100 in the proximal direction.

Similarly, as shown in FIG. 22, when the second shaft 5200 is moved distally relative to the first shaft 5100 through a predetermined distance, the second surface 5238 of the shoulder 5234 is configured to engage the shoulder 5139 of the spring pocket 5138. In this manner, the shoulder 5234 of the second shaft 5200 can limit the axial movement of the second shaft 5200 within the first shaft 5100 in the distal direction. Accordingly, the axial position of the retaining ring 5150 within the spring pocket 5138 and the axial position of shoulder 5234 on the second shaft 5200 cooperatively define a predetermined range of axial motion of the second shaft 5200 relative to the first shaft 5100.

As described above, the first shaft 5100 can be rotated about the second shaft 5200 to move the first shaft 5100 and the second shaft 5200 between a first configuration (FIG. 23) and a second configuration (FIG. 24). In the first configuration, the distal end surface 5215 of the second shaft 5200 is disposed outside of the first shaft 5100. Said another way, in the first configuration, the distal end surface 5215 of the second shaft 5200 is spaced apart from the distal end surface 5115 of the first shaft 5100 by a first distance D1. In the second configuration, the distal end surface 5215 of the second shaft 5200 is disposed within the first shaft 5100. Said another way, in the second configuration, the distal end surface 5215 of the second shaft 5200 is spaced apart from the distal end surface 5115 of the first shaft 5100 by a second distance D2. As described in more detail herein, this arrangement allows the nut 5610 to be maintained in a constant position within the nut engagement portion 5110 of first shaft 5100 and the screw engagement portion 5210 of the second shaft 5200 to be maintained in a constant position within the bone screw 5650, when the first shaft 5100 is rotated about the second shaft 5200. Said another way, this arrangement allows the nut 5610 to be maintained in a constant position within the nut engagement portion 5110 of first shaft 5100 and the screw engagement portion 5210 of the second shaft 5200 to be maintained in a constant position within the bone screw 5650, when nut 5610 is threadedly moved relative to the bone screw 5650 using the insertion tool 5000.

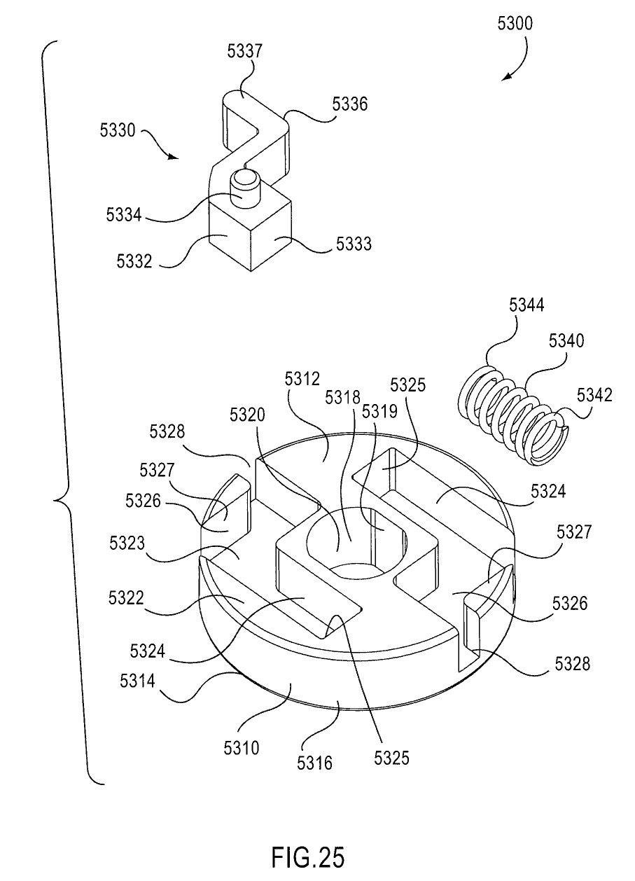

As shown in FIG. 25, the locking mechanism 5300 includes a lock housing 5310, a lock tab 5330, and a lock spring 5340. The lock tab 5330 includes a first portion 5332 and a second portion 5336. The first portion 5332 of the lock tab 5330 includes a spring engagement surface 5333 and a protrusion 5334. The second portion of the lock tab 5330 includes a protrusion 5337. The lock spring 5340 includes a first end 5342 and a second end 5344.

The lock housing 5310 includes an outer surface 5316, an inner surface 5318 (see FIG. 25), a proximal end surface 5312, and a distal end surface 5314. The outer surface 5316 defines an opening 5328 through which a portion of the lock tab 5330 can be disposed, as described in more detail below. The outer surface 5316 has a circular shape and is configured to fit within the splined portion 5137 of the actuation portion 5130 of the first shaft 5100. In this manner, when the locking mechanism 5300 is an unlocked configuration, as described in more detail below, the lock housing 5310 can rotate within splined portion 5137 of the first shaft 5100 about the longitudinal axis A_{L1}.

The inner surface 5318 of the lock housing 5310 includes two flatted portions 5319 and defines a lumen 5320. The flatted portions 5319 of the inner surface 5318 correspond to the two flatted surfaces 5242 of the proximal end portion 5202 of the second shaft 5200. In this manner, the proximal end portion 5202 of the second shaft 5200 can be disposed within the lumen 5320 such that the lock housing 5310 cannot rotate relative to the second shaft 5200. Said another way, this arrangement allows the lock housing 5310 and the second shaft 5200 cooperatively rotate within the first shaft 5100 when the locking mechanism 5300 is in the unlocked configuration.

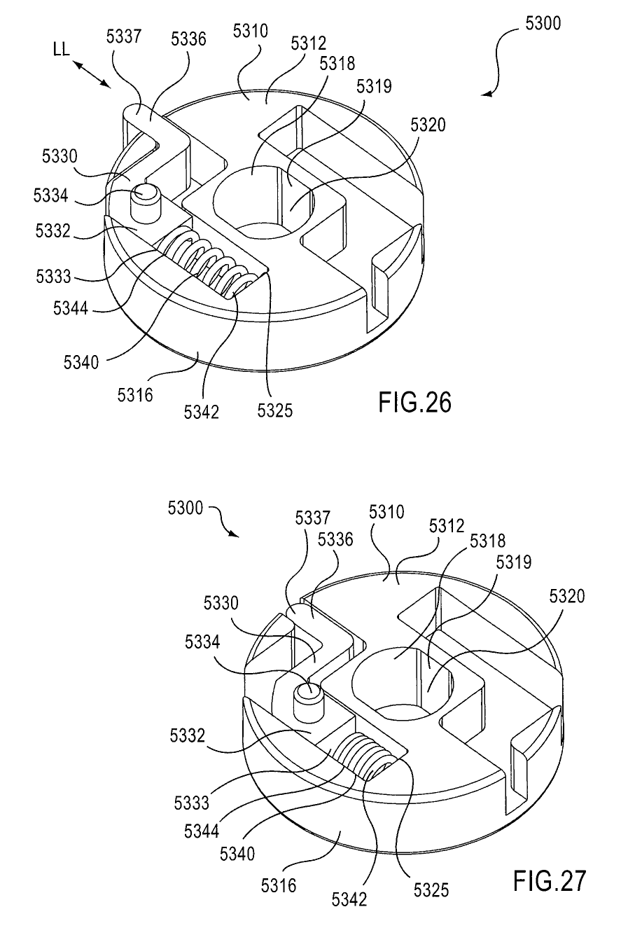

The proximal end surface 5312 of the lock housing 5310 includes a side wall 5322 that defines a channel 5323. Although FIGS. 25-27 show two channels 5323 that are arranged symmetrically on the proximal end surface 5312, the functionality of the lock housing 5310 can be achieved with a single channel 5323. The channel 5323 includes a first portion 5324 and a second portion 5326. As shown in FIG. 26, the lock tab 5330 is movably disposed within the channel 5323 such that the first portion 5332 of the lock tab 5330 is within the first portion 5324 of the channel 5323 and at least a portion of the second portion 5336 of the lock tab 5330 is within the second portion 5326 of the channel 5323. The protrusion 5334 of the lock tab 5330 is spaced proximally apart from the proximal end surface 5312 of the lock housing 5310. As described in more detail herein, the protrusion 5334 is configured to be received within a portion of the actuator 5400 such that movement of the actuator 5400 causes the lock tab 5330 to move within the channel 5323, as indicated by the arrow LL in FIG. 26.

The lock tab 5330 can move within the channel 5323 to move the locking mechanism 5300 between a locked configuration (FIG. 26) and an unlocked configuration (FIG. 27). When the locking mechanism 5300 is in the locked configuration, the protrusion 5337 of the lock tab 5330 is disposed through the opening 5328 of the lock housing 5310. Moreover, a portion of the lock tab 5330 is in contact with a stop surface 5327 of the channel 5323. Said another way, when the locking mechanism 5300 is in the locked configuration, the protrusion 5337 of the lock tab 5330 is spaced radially apart from the outer surface 5316 of the lock housing 5310. Moreover, when the when the locking mechanism 5300 is in the locked configuration, the protrusion 5337 of the lock tab 5330 is disposed within one of the splines 5137 defined by the first shaft 5100 (note that the splines 5137 are not shown in FIGS. 26 and 27). Accordingly, when the locking mechanism 5300 is in the locked configuration, rotation of the lock housing 5310 and the second shaft 5200 within the first shaft 5100 is prevented.

As shown in FIG. 27, when the locking mechanism 5300 is in the unlocked configuration, the protrusion 5337 of the lock tab 5330 is disposed within the channel 5323 of the lock housing 5310. Said another way, when the locking mechanism 5300 is in the unlocked configuration, the protrusion 5337 of the lock tab 5330 is spaced apart from the splines 5137 defined by the first shaft 5100. Accordingly, when the locking mechanism 5300 is in the unlocked configuration, the lock housing 5310 and the second shaft 5200 are able to freely rotate within the first shaft 5100.

The first portion 5324 of the channel 5323 terminates in a spring engagement surface 5325. The lock spring 5340 is disposed within the first portion 5324 of the channel 5323 such that the first end 5342 of the lock spring 5340 is in contact with the spring engagement surface 5325 and the second end 5344 of the lock spring 5340 is disposed against the spring surface 5333 of the lock tab 5330. In this manner, the lock tab 5330 is biased within the channel 5323 such that the locking mechanism 5300 is in the locked configuration. In other embodiments, the lock spring 5340 can be arranged such that the locking mechanism 5300 is biased in the unlocked configuration.

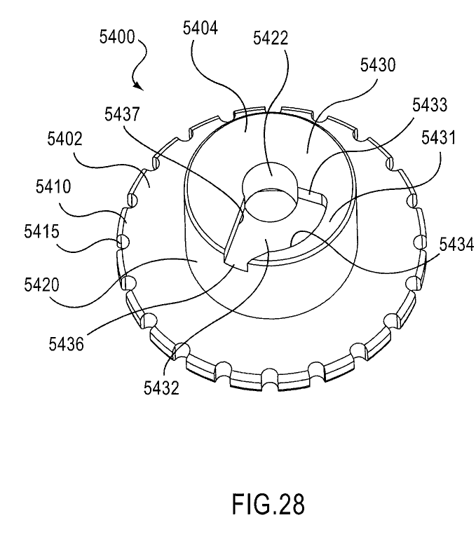

As shown in FIGS. 28-30, the actuator 5400 includes a proximal portion 5402 and a distal portion 5404. The actuator 5400 defines a lumen 5422 having a longitudinal axis A_{L}. The lumen 5422 is sized such that the actuator 5400 can be disposed about the proximal end portion 5202 of the second shaft 5200. Unlike the lumen 5320 of the lock housing 5300, the lumen 5422 is substantially circular and devoid of flatted portions such that the actuator 5400 can rotate relative to the second shaft 5200 and/or the locking mechanism 5300. Said another way, this arrangement allows the actuator 5400 to rotate about the longitudinal axis A_{L2} independently from the rotation of the second shaft 5200 and/or the locking mechanism 5300.

The proximal portion 5402 of the actuator 5400 includes a flange 5410 having a substantially circular outer surface 5412. The outer surface 5412 of the flange 5410 includes multiple alternating recesses 5415. In this manner, the outer surface 5412 of the flange 5410 can be grasped and/or manipulated by the user, for example, to rotate the actuator 5400 about the first shaft 5100 and/or the second shaft 5200. Although the outer surface 5412 is shown as including multiple recesses, in other embodiments, the outer surface 5412 can include any suitable topographical features to aid in the manipulation of the actuator 5400 For example, in some embodiments, the outer surface 5412 can be knurled, cross-hatched or the like.

The flange 5410 of the actuator 5400 includes a proximal end surface 4512 that is configured to be disposed adjacent and/or engaged with the handle 5500, as shown in FIGS. 7 and 10. The proximal end surface 5412 defines a proximal opening 5418 that is in fluid communication with the lumen 5422. The proximal opening 5418 is configured to receive a distal protrusion 5520 of the handle 5500 such that the handle 5500 can be matingly disposed within a portion of the actuator 5400.

The distal portion 5402 of the actuator 5400 includes an outer surface 5420 and a distal end surface 5430. The outer surface 5420 has a substantially circular shape, and is configured to be received within the splined portion 5137 of the actuation portion 5130 of the first shaft 5100. Accordingly, the actuator 5400 can rotate within splined portion 5137 of the first shaft 5100 about the longitudinal axis A_{L1}. As shown in FIG. 10, the distal portion 5404 of the actuator 5400 is disposed within the first shaft 5100 proximally from the locking mechanism 5300.

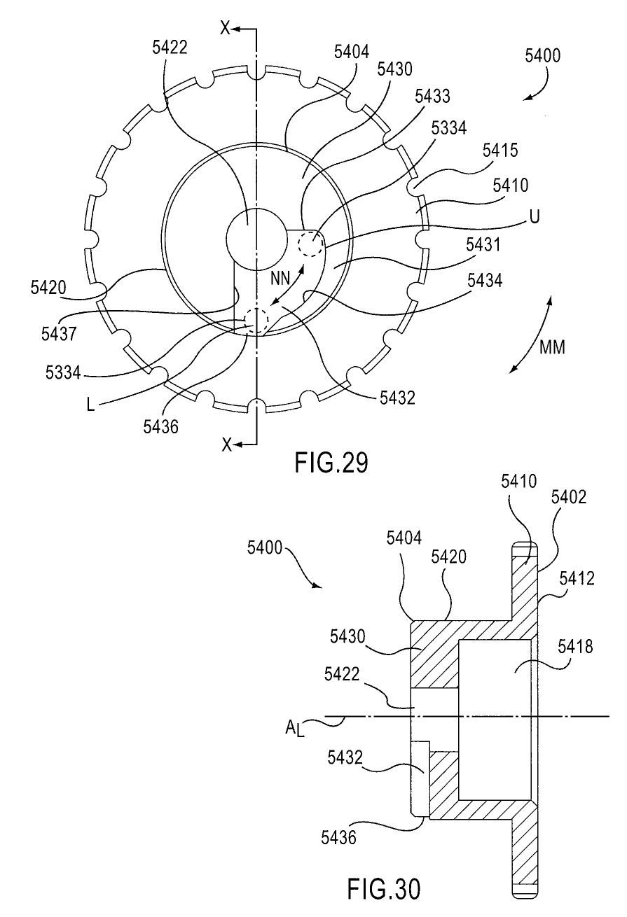

The distal end surface 5430 of the actuator 5400 includes a side wall 5431 that defines a recess 5432. The side wall 5431 also defines an opening 5436. The side wall 5431 includes a cam surface 5434, a first end surface 5433, and a second end surface 5437. The cam surface 5434 is a curved surface having a radius of curvature that is offset from the longitudinal axis A_{L} of the actuator 5400. In this manner, as shown in FIG. 29, the distance between the cam surface 5434 and center of the lumen 5422 (i.e., the longitudinal axis A_{L}) varies circumferentially.

As shown in FIGS. 10 and 31, the distal end surface 5430 of the actuator 5400 is disposed adjacent and/or in contact with the proximal end surface 5312 of the lock housing 5310. Moreover, the protrusion 5334 of the lock tab 5330 is disposed within the recess 5432 of the actuator 5400 such that a portion of the protrusion 5334 is in contact with a portion of the cam surface 5434. FIG. 29 shows the protrusion 5334 as disposed within the recess 5432 in dashed lines. Accordingly, when the actuator 5400 is rotated about the longitudinal axis A_{L} relative to the second shaft 5200 and the locking mechanism 5300, as shown by the arrow MM in FIG. 29, the cam surface 5434 slides relative to the protrusion 5334 of the lock tab 5330 as shown by the arrow NN in FIG. 29. In this manner, the lock tab 5330 can be moved within the channel 5323 as shown by the arrow LL in FIG. 26 to move the locking mechanism 5300 between the locked configuration and the unlocked configuration. Said another way, the rotation of the actuator 5400 causes the lock tab 5330 to translate within the channel 5323. Said yet another way, the actuator 5400 is configured to move the locking mechanism 5300 between the locked configuration and the unlocked configuration by rotating about the longitudinal axes A_{L1}, A_{L2}.

When the locking mechanism 5300 is in the locked configuration, the protrusion 5334 of the lock tab 5330 is disposed within the recess 5432 of the actuator 5400 adjacent the opening 5436, as indicated by POS L. Accordingly, when the user attempts to rotate actuator 5400 further in the counter-clockwise direction (when viewing FIG. 31), a portion of the protrusion 5334 contacts the second end surface 5437, thereby preventing further counter-clockwise rotation. Similarly, when the locking mechanism 5300 is in the unlocked configuration, the protrusion 5334 of the lock tab 5330 is disposed within the recess 5432 of the actuator 5400 as indicated by POS U. Accordingly, when the user attempts to rotate actuator 5400 further in the clockwise direction (when viewing FIG. 31), a portion of the protrusion 5334 contacts the first end surface 5433, thereby preventing further clockwise rotation. In this manner, the first end surface 5433 and the second end surface 5437 cooperatively serve to limit the rotational motion of the actuator 5400. More particularly, the first end surface 5433 and the second end surface 5437 are spaced apart such that the actuator 5400 is limited to approximately 90 degrees of rotation (i.e., one-quarter turn). In other embodiments, the first end surface 5433 and the second end surface 5437 are spaced apart such that the actuator 5400 is limited to any desired amount of rotation (e.g., one-half turn, three-quarters turn, etc.).

As shown in FIGS. 10 and 31, the proximal end 5182 of the spring 5180 is disposed against the distal end surface 5314 of the lock housing 5310. Accordingly, the spring 5180 biases the lock housing 5310 proximally such that contact between the proximal end surface 5312 of the lock housing 5310 and the distal end surface 5430 of the actuator 5400 is maintained. In this manner, the protrusion 5334 remains within the recess 5432 of the actuator 5400 when the second shaft 5200 moves within the first shaft 5100, as described above.

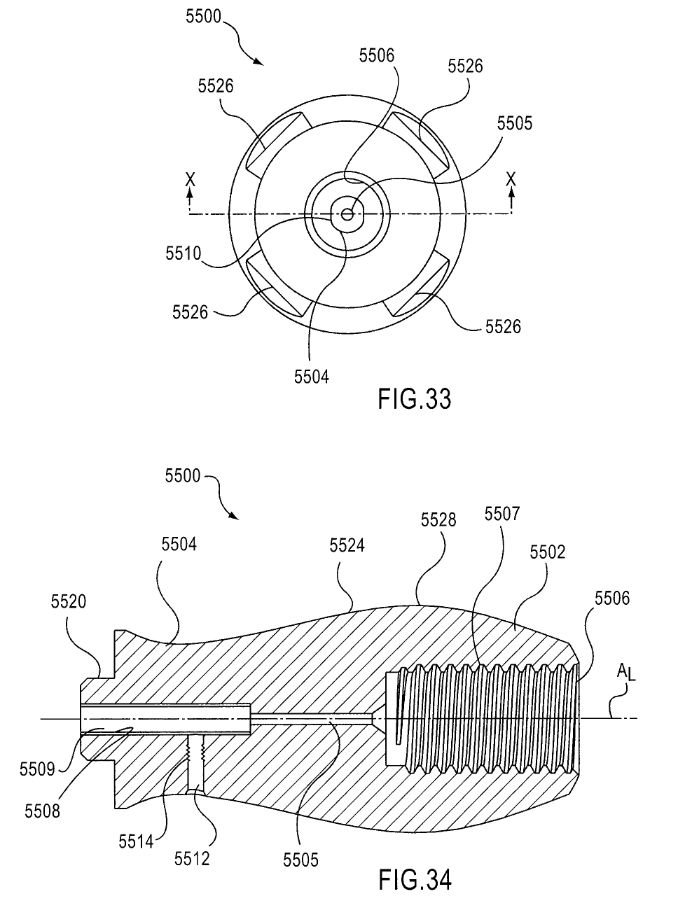

As shown in FIGS. 32-34, the handle 5500 includes a proximal portion 5502 and a distal portion 5504. The handle 5500 defines a lumen 5505 having a longitudinal axis A_{L}. As shown in FIG. 10 and described in more detail herein, the handle 5500 is configured to be coupled to the second shaft 5200 such that the longitudinal axis A_{L} of the lumen 5505 is substantially concentric with the longitudinal axis A_{L2} of the lumen 5220 of the second shaft 5200. In this manner, the guide wire 5550 can be disposed through the lumen 5505 and the lumen 5220.

The handle 5500 includes an outer surface 5524. A portion of the outer surface 5524 is bulb-shaped, and a portion of the outer surface 5524 includes multiple flats 5526. In this manner, the outer surface 5524 of the handle 5500 is configured to be grasped and/or manipulated by the user, for example, to rotate the first shaft 5100 and/or the second shaft 5200 about the longitudinal axes A_{L1} and/or A_{L2}. Although the outer surface 5524 is shown as including multiple flats 5526, in other embodiments, the outer surface 5134 can include any suitable topographical features to aid in the manipulation of the handle 5500.

The proximal portion 5502 of the handle 5500 includes a proximal opening 5506 that has a threaded portion 5507. Said another way, the proximal portion 4402 of the handle 5500 defines an opening 5506 having female threads 4507. The threaded portion 5507 of the handle 5500 corresponds to the threaded portion 5562 of the guide wire 5550 such that when a portion of the guide wire 5500 is disposed within the handle 5500, the threaded portion 5507 of the handle 5500 can be matingly engaged with the threaded portion 5562 of the guide wire 5550. Said yet another way, the thread pitch of the threaded portion 5507 of the handle 5500 is substantially the same as the thread pitch of the threaded portion 5562 of the guide wire 5550.

The distal portion 5504 of the handle 5500 includes a distal protrusion 5520 and an inner surface 5508. As described above, the distal protrusion 5520 is configured to be received within the proximal opening 5418 of the actuator 5400. The inner surface 5508 defines a distal opening 5509 configured to receive a portion of the proximal portion 5202 of the second shaft 5200. Moreover, the inner surface 5508 includes two flatted portions 5510 that correspond to the two flatted surfaces 5242 of the proximal portion 5202 of the second shaft 5200. In this manner, when the handle 5500 is disposed about the second shaft 5200, rotation of the handle 5500 causes simultaneous rotation of the second shaft 5200.

The handle 5500 further defines a transverse lumen 5512 that is substantially normal to the longitudinal axis A_{L} of the lumen 5505. The transverse lumen 5512 intersects the distal opening 5509 adjacent one of the flatted portions 5510. The transverse lumen includes a female threaded portion 5514 that corresponds with a threaded portion of a set screw 5515. Accordingly, when the handle 5500 is disposed about the second shaft 5200, the set screw 5515 can be threadedly advanced within the transverse lumen 5512 until a portion of the set screw 5515 engages a portion of one of the flatted surfaces 5242 of the proximal portion 5202 of the second shaft 5200. In this manner, the handle 5500 can be fixedly coupled to the second shaft 5200.

As shown in FIGS. 10 and 36, the guide wire 5550 includes a proximal end portion 5552 and a distal end portion 5554. The distal end portion 5554 includes a tapered tip 5556 configured to pierce, dilate and or distract bodily tissue. In some embodiments, for example, the tapered tip 5556 can be configured to pierce bone tissue. Although the distal end portion 5554 of the guide wire 5550 is shown as being devoid of threads, in some embodiments, the distal end portion 5554 of the guide wire 5550 can include a threaded portion configured to assist in defining a passageway within the bone tissue when the guide wire 5550 is advanced into the bone tissue, as described in more detail herein.

The proximal end portion 5552 of the guide wire 5550 includes an actuation portion 5560. The actuation portion 5560 includes a flange 5564, proximal end surface 5566, and a threaded portion 5562. The flange 5564 includes multiple recesses along the circumference of the flange 5564. In this manner, the flange 5564 can be grasped and/or manipulated by the user, for example, to rotate the guide wire 5550 within the handle 5500, as described in more detail below. Although the flange 5564 is shown as including multiple recesses, in other embodiments, the flange 5564 can include any suitable topographical features to aid in the manipulation of the guide wire 5550.

The threaded portion 5562 of the actuation portion 5560 is disposed on an outer surface of the actuation portion 5560. Said another way, the threaded portion 5562 includes male threads on the outer surface of the actuation portion 5560. As shown in FIG. 10, at least a portion of the guide wire 5550 is disposed within the lumen 5505 of the handle 5500 and/or the lumen 5220 of the second shaft 5200 such that the threaded portion 5562 of the actuation portion 5560 is engaged with the threaded portion 5507 of the handle 5500. Said another way, the threaded portion 5562 of the actuation portion 5560 corresponds to the threaded portion 5507 of the handle 5500 such that when a portion of the guide wire 5550 is disposed within the lumen 5505 and/or the lumen 5220, the threaded portion 5562 of the actuation portion 5560 can be matingly engaged with the threaded portion 5507 of the handle 5500.

The axial position of the guide wire 5550 within the second shaft 5200 can be adjusted by rotating the actuation portion 5562 of the guide wire 5550 within the handle 5500, as indicated by the arrow OO in FIG. 36. Said another way, the guide wire 5550 can be moved axially within the second shaft 5200 in a controlled and/or incremental manner by rotating the guide wire 5550 within the handle 5500. In this manner, the position of the distal tip 5556 of the guide wire 5550 relative to the distal end surface 5215 of the second shaft 5200 and/or the distal end 5654 of the bone screw 5650 can be selectively adjusted.

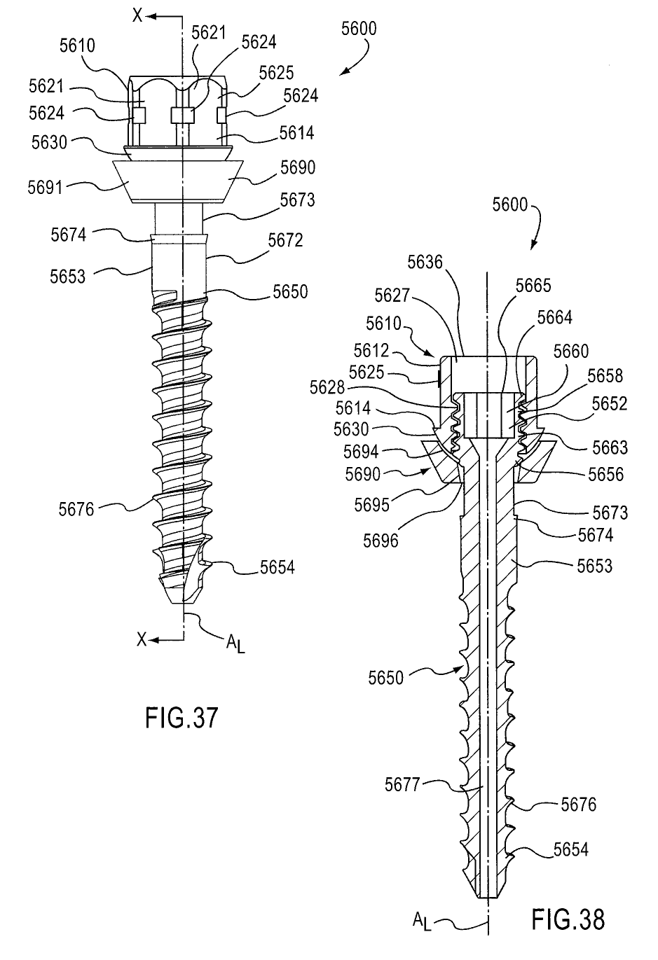

As shown in FIGS. 37 and 38, the bone fixation device 5600 includes a nut 5610, a bone screw 5650, and a washer 5690. The bone screw 5650 includes a proximal end portion 5652, a distal end portion 5654, and a central portion 5653 therebetween. The bone screw 5650 defines a lumen 5677 having a longitudinal axis A_{L}. As shown in FIG. 10 and described in more detail herein, the bone fixation device 5600 is configured to be selectively coupled to the insertion tool 5000 such that the longitudinal axis A_{L} of the lumen 5677 is substantially concentric with the longitudinal axis A_{L2} of the lumen 5220 of the second shaft 5200 and/or the longitudinal axis of the guide wire 5550. In this manner, the guide wire 5550 can be disposed within and/or through the lumen 5677 of the bone screw 5650.

The distal end portion 5654 of the bone screw 5650 includes a self-tapping tip and threaded portion 5676. The self-tapping tip and the threaded portion 5676 can have any suitable geometric characteristics (e.g., thread pitch, helix angle, etc.) for being threadedly disposed within bone tissue. In this manner, the bone screw 5650 can be threaded into a targeted bone tissue without requiring a threaded passageway within the targeted bone tissue.

The proximal end portion 5652 of the bone screw includes an engagement portion 5656. The engagement portion 5656 includes a side wall 5658 having a proximal end surface 5665. The side wall 5658 defines a hexagonal shaped recess 5660 corresponding to the hexagonal shaped portions of the screw engagement portion 5210 of the second shaft 5200 (see e.g., FIG. 17). In this manner, the engagement portion 5656 of the bone screw 5650 can receive a portion of the screw engagement portion 5210 of the second shaft 5200 such that rotation of the second shaft 5200 about its longitudinal axis A_{L2} results in rotation of the bone screw 5650.

The side wall 5658 of the engagement portion 5656 includes a threaded portion 5663 and a flange 5664. The threaded portion 5663 includes male threads on the outer surface of the side wall 5658 that correspond to the threaded portion 5628 of the nut 5610. Said another way, the thread pitch of the threaded portion 5663 of the bone screw 5650 is substantially the same as the thread pitch of the threaded portion 5628 of the nut 5610. Moreover, the thread pitch of the threaded portion 5663 of the bone screw 5650 and/or the thread pitch of the threaded portion 5628 of the nut 5610 is substantially the same as the thread pitch of the threaded portion 5126 of the first shaft 5100 and/or the threaded portion 5232 of the second shaft 5200. Accordingly, when the second shaft 5200 is rotated within the first shaft 5100, the distance through which the second shaft 5200 moves axially relative to the first shaft 5100 is the same as the distance through which the nut 5610 moves axially relative to the bone screw 5650.

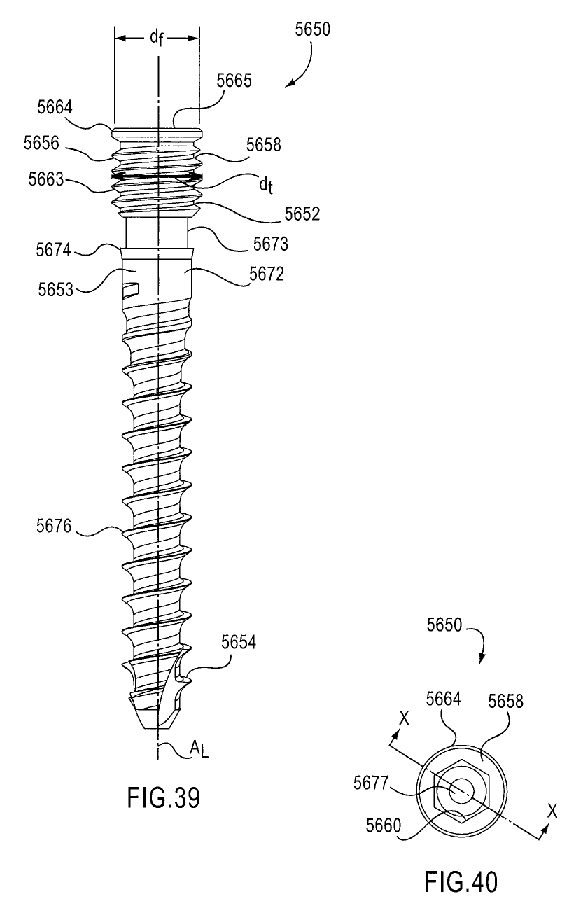

As shown in FIGS. 39 and 44, the flange 5664 of the engagement portion 5656 has an outer diameter d_{f} that is greater than the outer diameter dₜ of the threaded portion 5663 of the bone screw 5650 and inner diameter dₙ of the threaded portion 5628 of the nut 5610. This arrangement prevents the nut 5610 from being removed from the engagement portion 5656 of the bone screw 5650 in a proximal direction. Accordingly, the nut 5610 can be threaded onto the engagement portion 5656 of the bone screw 5650 by first disposing the nut 5610 about the distal end portion 5654 of the bone screw 5650 and then moving the nut 5610 proximally until the proximal portion of the threaded portion 5628 of the nut 5610 is engaged with the distal portion of the threaded portion 5663 of the bone screw 5650.

The central portion 5653 of the bone screw 5650 includes a non-threaded outer surface 5672 disposed between the threaded portion 5676 of the distal end portion 5654 and the threaded portion 5663 of the engagement portion 5656. The outer surface 5672 defines a recess 5673 within which at least a portion of the washer 5690 is disposed. The outer surface 5672 also includes a tapered protrusion 5674. As described in more detail below, the tapered protrusion 5674 is configured to retain the washer 5690 within the recess 5673, while allowing the washer 5690 to move within the recess. Said another way, the tapered protrusion 5674 is configured to limit the movement of the washer 5690 in the distal direction within the recess 5673.

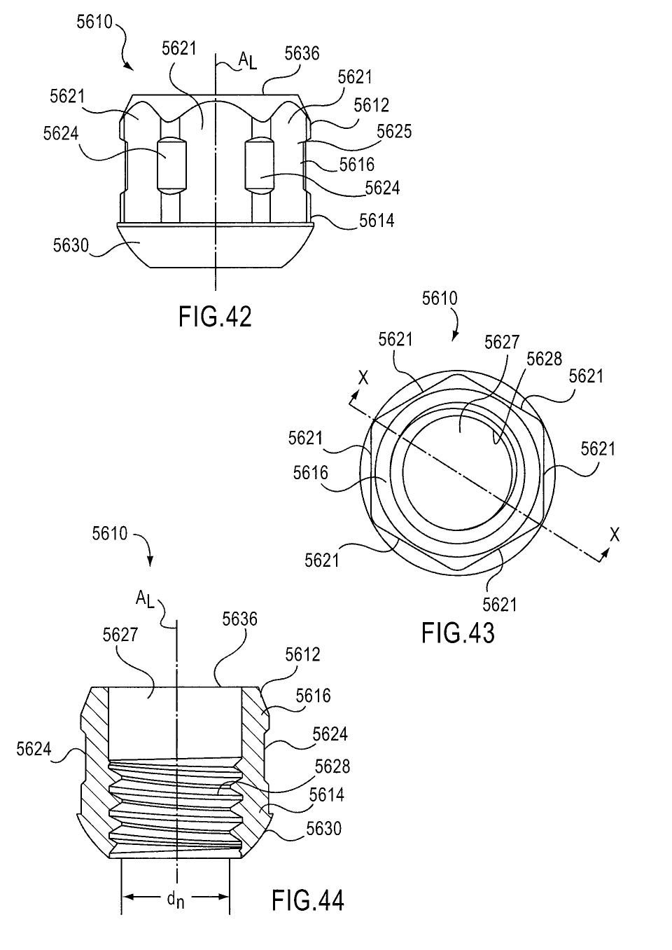

As shown in FIGS. 42-44, the nut 5610 includes a proximal end portion 5612 and a distal end portion 5614. The nut 5610 defines a lumen 5627 having a longitudinal axis A_{L}. As shown in FIGS. 37 and 38, the nut 5610 is configured to be threadedly coupled to the bone screw 5650 such that the longitudinal axis A_{L} of the lumen 5627 is substantially concentric with the longitudinal axis A_{L} of the lumen 5677 of the bone screw 5650.

The proximal end portion 5612 of the nut 5610 includes an engagement portion 5625. The engagement portion 5625 includes a side wall 5616 and a proximal end surface 5636. The side wall 5616 has an outer surface that includes six hexagonal flats 5621 corresponding to the hexagonal-shaped inner surface 5113 of the nut engagement portion 5110 of the first shaft 5100. In this manner, the engagement portion 5625 of the nut 5610 can be disposed within the nut engagement portion 5110 of the first shaft 5100 such that rotation of the first shaft 5100 about its longitudinal axis A_{L1} results in rotation of the nut 5610. Said another way, the engagement portion 5625 of the nut 5610 can be disposed within the nut engagement portion 5110 of the first shaft 5100 such that rotational movement of the nut 5610 relative to the first shaft 5100 is limited.

The outer surface of the side wall 5616 defines multiple grooves 5624 disposed substantially normal to the longitudinal axis A_{L} of the nut 5610. Said another way, the apex of each of the flats 5621 defines a groove 5624. The grooves 5624 are configured to receive a portion of a nut retention member 5160. As discussed above, a portion of the nut retention member 5160 is also disposed within the groove 5119 of the nut engagement portion 5110 of the first shaft 5100. In this manner, the nut retention member 5160 can selectively retain the nut 5610 within the nut engagement portion 5110 of the first shaft 5100. Said another way, the nut retention member 5160 can limit movement of the nut 5610 relative to the first shaft 5100 along the longitudinal axis A_{L1}.

The nut retention member 5160 can be any suitable retention member for selectively retaining the nut 5610 within the nut engagement portion 5110 of the first shaft 5100. For example, in some embodiments, the nut retention member can be a snap ring, a circular-shaped coiled spring, an elastic member or the like. In some embodiments, for example, the nut retention member 5160 can be a canted coiled spring that can be compressed radially and/or axially to be selectively retained within the groove 5119 of the nut engagement portion and/or the grooves 5624 of the nut. In some embodiments, the nut retention member 5160 can be a canted coiled spring produced by Bal Seal Engineering Inc.

The distal end portion 5614 of the nut 5610 includes a threaded portion 5628 within the lumen 5627. Said another way, the distal end portion 5614 of the nut 5610 defines a female threaded portion 5628. As described above, the threaded portion 5628 of the nut 5610 corresponds to the threaded portion 5663 of the bone screw 5650, the threaded portion 5126 of the first shaft 5100 and/or the threaded portion 5232 of the second shaft 5200.

The outer surface the distal end portion 5614 of the nut 5610 includes a curved surface 5630 configured to engage the washer 5690. More particularly, the curved surface 5630 of the nut 5610 corresponds to the curved surface 5694 of the washer 5690, such that a portion of washer 5690 can be matingly disposed about the curved surface 5630 of the nut 5610. Said another way, a radius of curvature of the curved surface 5630 of the nut 5610 is substantially the same as a radius of curvature of the curved surface 5694 of the washer 5690. In this manner, when the nut 5610 is tightened on the bone screw 5650, the clamping load is transferred in a uniform and/or spatially distributed fashion to the washer 5690. Moreover, as described in more detail below, this arrangement allows the washer 5690 to rotate relative to the nut 5610 and/or the bone screw 5650 about an axis substantially normal to the longitudinal axis A_{L} of the nut 5610.

As shown in FIG. 45, the washer 5690 includes an outer surface 5691, an inner surface 5693, and a distal end surface 5692. Although the outer surface 5691 is shown as being conically shaped, the outer surface 5691 can have any suitable shape. The distal end surface 5692 is configured to engage the targeted bone tissue (not shown in FIG. 42) when the nut 5610 is tightened on the bone screw 5650.

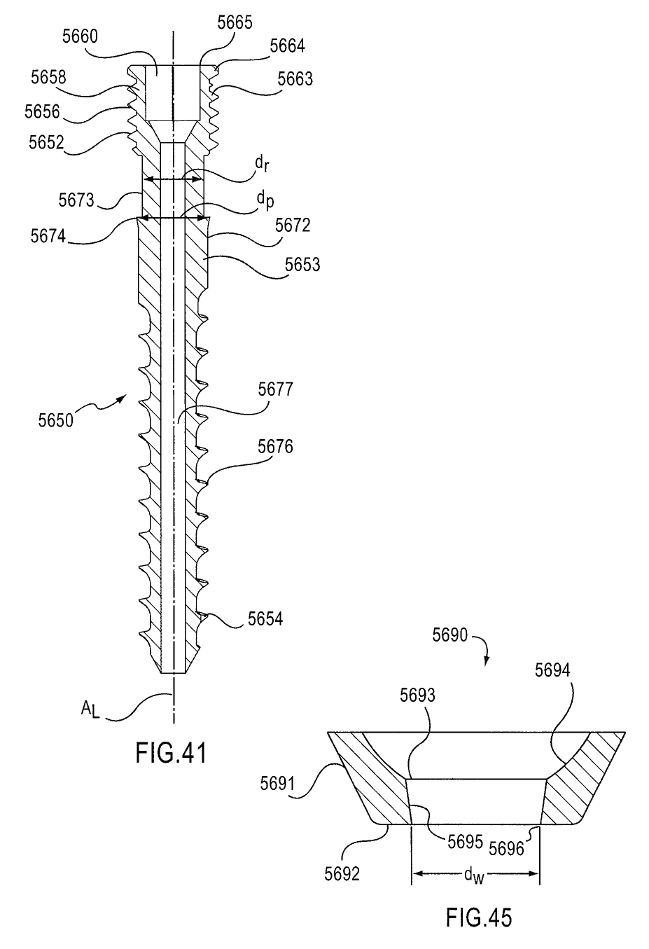

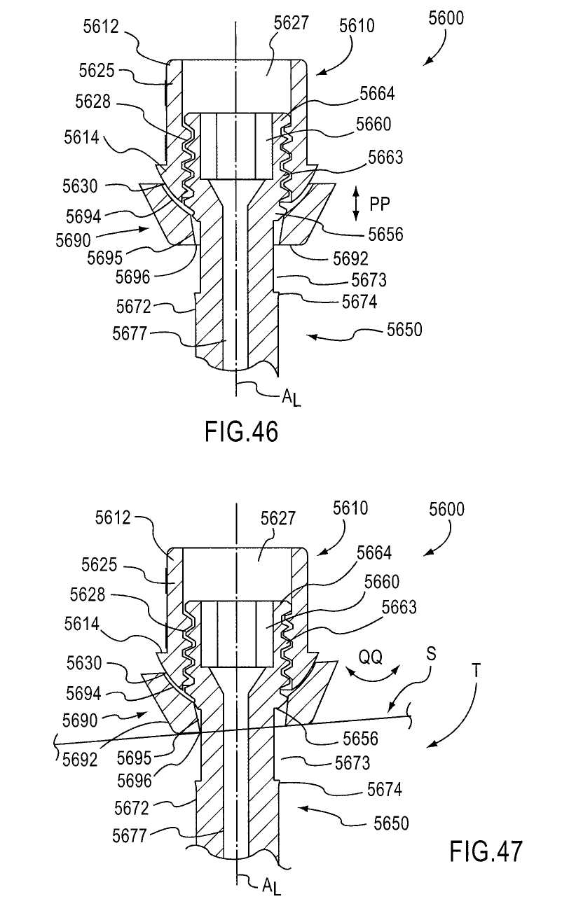

The inner surface 5693 of the washer includes a curved portion 5694 and a tapered portion 5695. The tapered portion 5695 includes a protrusion 5696 adjacent the distal end surface 5692 of the washer 5690. The protrusion 5696 of the washer 5690 has an inner diameter d_{w} that is less than an outer diameter dₚ (see FIG. 41) of the protrusion 5674 of the bone screw 5650. Moreover, the inner diameter d_{w} of the protrusion 5696 is greater than an outer diameter dᵣ (see FIG. 41) of the recess 5673. Accordingly, when the washer 5690 is disposed within the recess 5673 of the bone screw 5650, the washer 5690 can move axially about the bone screw, as shown by the arrow PP in FIG. 46, until the protrusion 5696 of the washer 5690 contacts the protrusion 5674 of the bone screw 5650 or the threaded portion 5663 of the bone screw 5650. Said another way, the axial movement of the washer 5690 within the recess 5673 is limited by the protrusion 5674 of the bone screw 5650 or the threaded portion 5663 of the bone screw 5650. The washer 5690 can be disposed within the recess 5673 by first disposing the washer 5690 about the distal end portion 5654 of the bone screw 5650 and then moving the washer 5690 proximally until the protrusion 5696 of the washer 5690 is snap-fit over the protrusion 5674 of the bone screw 5650.

As shown by the arrow QQ in FIG. 47, the washer 5690 can rotate relative to the nut 5610 and/or the bone screw 5650 about an axis substantially normal to the longitudinal axis A_{L} of the bone fixation device 5600. Accordingly, when the bone fixation device 5600 is disposed within and/or against a targeted bone tissue T, the washer 5690 can move relative to the bone screw 5650 such that the distal end surface 5692 of the washer 5690 is substantially parallel to the surface S of the targeted bone tissue T. Said another way, when the bone fixation device 5600 is disposed within and/or against a targeted bone tissue T, the washer 5690 can move relative to the bone screw 5650 such that the distal end surface 5692 of the washer 5690 is flush against the surface S of the targeted bone tissue T. In this manner, the washer 5690 can substantially evenly distribute the clamping load applied by the bone fixation device 5600 regardless of the angular offset between the passageway within the targeted bone tissue T and the surface S of the targeted bone tissue. Said another way, this arrangement allows the washer 5690 to be disposed substantially flush against the surface S of the targeted bone tissue T without requiring a counter bore and/or a countersink in the surface S of the targeted bone tissue T.

The range of rotational motion of the washer 5690 can be limited based on when the protrusion 5696 of the washer 5690 contacts the portion of the outer surface 5672 of the bone screw 5650 that defines the recess 5673. Said another way, the greater the difference between the inner diameter d_{w} of the protrusion 5696 and the outer diameter dᵣ (see FIG. 41) of the recess 5673, the greater the range of rotational motion of the washer 5690. In this manner, the washer 5690 and the bone screw 5650 can be configured to have a predetermined range of relative motion about the axis substantially normal to the longitudinal axis A_{L} of the bone fixation device 5600.

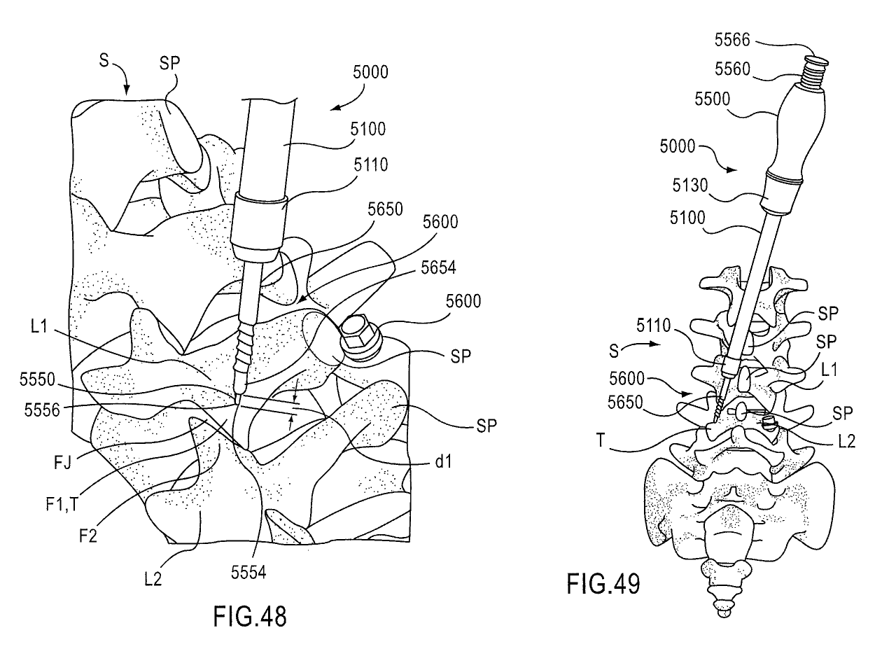

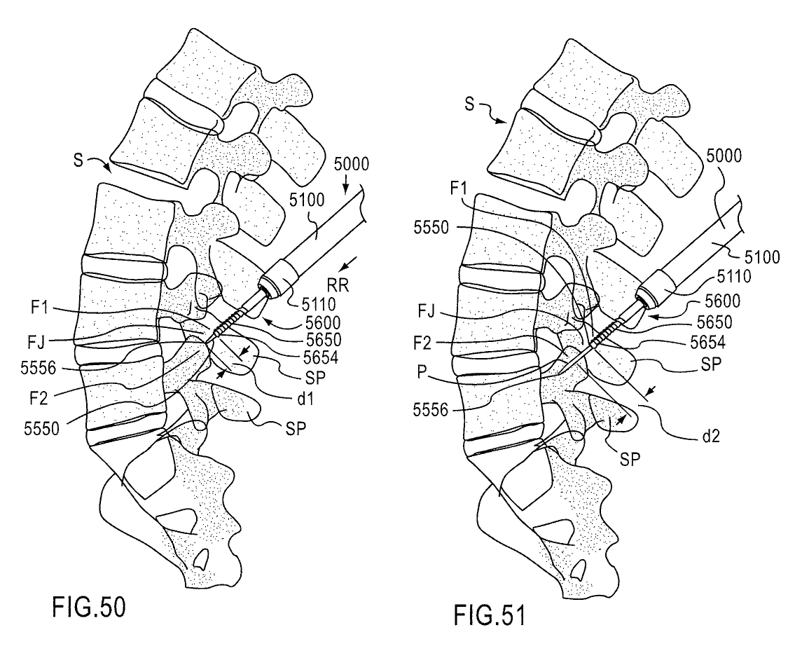

FIGS. 48 through 54 are various views showing a method of inserting the bone fixation device 5600 into a portion of the spine S using the insertion tool 5000. For the sake of clarity, the skin and surrounding tissue of the patient's body is not depicted in FIGS. 48 through 54. In use, the bone fixation device 5600 is coupled to the insertion tool 5000 prior to inserting the bone fixation device 5600 into the body, as described above. As shown in FIG. 10, the guide wire 5550 can be disposed within the handle 5500 such that the distal tip 5556 of the guide wire extends beyond the distal end portion 5654 of the bone screw 5650 by a first distance d1 (see e.g., FIGS. 48 and 50). In some embodiments, the distal tip 5556 of the guide wire 5550 can extend beyond the distal end portion 5654 of the bone screw 5650 by approximately 2 to 8 mm. In some embodiments, the first distance d1 can be less than the desired length of the passageway to be defined within the bone tissue. As described in more detail herein, in such embodiments, the guide wire 5550 can be advanced into the bone tissue in an incremental fashion, by adjusting the distance between the distal tip 5556 of the guide wire and the distal end portion 5654 of the bone screw 5650 while the bone fixation device 5600 and the insertion tool 5000 are disposed within the body. In this manner, the likelihood that the guide wire 5550 will buckle when the guide wire 5550 is advanced into the bone tissue can be reduced or minimized.

As shown in FIGS. 48 and 49, the bone fixation device 5600 and a distal portion of the insertion tool 5000 are inserted into the body via a skin incision (not shown in FIGS. 48 and 49) adjacent the target location T. Although the insertion tool 5000 is shown in FIGS. 48 and 49 as being inserted via a substantially midline incision, in other embodiments, the insertion tool 5000 can be inserted via an incision lateral to the spinous process SP (e.g., an ipsilateral incision or a contralateral incision). The incision can be, for example, approximately 15 mm in length. The distal tip 5556 of the guide wire 5550 is then disposed against the surface of the target location T (i.e., the guide wire 5550 is “docked” against the target location T). As shown in FIGS. 48 and 49, in this example, the target location T is the inferior facet F1 of the superior level (shown generally as L1).

The guide wire 5550 is then advanced into the inferior facet F1 of the superior level to define a portion of the passageway within the bone tissue, as shown by the arrow RR in FIG. 50. In this manner, the passageway within the bone tissue can be defined while the bone fixation device 5600 is disposed within the body. In some embodiments, for example, the guide wire 5550 can be advanced by striking the proximal end surface 5566 of the guide wire 5550 (see e.g., FIG. 49) with a hammer. In other embodiments, for example, the guide wire 5550 can be advanced by rotating the guide wire such that the distal tip 5556 can be rotatably advanced into the bone tissue. For example, in some embodiments, the distal end portion 5554 of the guide wire 5550 can include a threaded portion to assist in advancing the guide wire 5550 within the bone tissue.

In some embodiments, the guide wire 5550 can be advanced into the bone tissue in an incremental fashion. For example, as shown in the lateral view depicted in FIG. 50, in some embodiments, the guide wire 5550 can be advanced the first distance d1 into the bone tissue. As described above, the first distance is the distance that the guide wire 5550 extends beyond the distal end portion 5654 of the bone screw 5650 prior to inserting the bone fixation device 5600 into the body. After the guide wire 5550 is advanced by the first distance d1 into the bone tissue, the distal end portion 5654 of the bone screw 5650 can be disposed against the inferior facet F1 of the superior level.

As shown in FIG. 51, the guide wire 5550 can then be moved axially within the second shaft (see e.g., FIG. 10) and the bone screw 5650, by rotating the guide wire 5550 within the handle 5500, as described above. Said another way, the distance between the distal tip 5556 of the guide wire and the distal end portion 5654 of the bone screw 5650 can be changed while the bone fixation device 5600 and the insertion tool 5000 are disposed within the body. In this manner, the distance between the distal tip 5556 of the guide wire 5550 and the distal end portion 5654 of the bone screw 5650 can be incrementally changed from the first distance d1 to a second distance d2 greater than the first distance d1. Accordingly, as guide wire 5550 is moved within the second shaft 5200, the distal end portion 5654 of the bone screw 5650 is disposed apart from the surface of the inferior facet F1 of the superior level by a distance approximately equal to the difference between the second distance d2 and the first distance d1. The guide wire 5550 can again be advanced into the bone tissue by striking the proximal end surface 5566 of the guide wire 5550 with a hammer until the distal end portion 5654 of the bone screw 5650 is again disposed against the inferior facet F1 of the superior level. In this manner, the guide wire 5550 can be advanced by the second distance d2 into the bone tissue. Such an incremental procedure can reduce the likelihood that the guide wire 5550 will buckle when being advanced into the bone tissue. In this manner, as shown in FIG. 51, the guide wire 5550 can be advanced to define the passageway through the inferior facet F1 of the superior level, across the facet joint FJ, through the superior facet F2 of the inferior level, and into the pedicle P of the inferior level (shown generally as L2).

Although the operations of moving the guide wire 5550 axially within the second shaft such that the distance between the distal tip 5556 of the guide wire 5550 and the distal end portion 5654 of the bone screw 5650 is increased to the second distance d2 and subsequently advancing the guide wire 5550 into the bone tissue are described above as being performed in sequentially, in other embodiments, these operations can be performed substantially simultaneously. For example, in some embodiments, the guide wire 5550 can then be moved axially within the second shaft while the distal end portion 5654 of the bone screw 5650 is maintained in contact with the surface of the inferior facet F1 of the superior level. In this manner, when the distal tip 5556 of the guide wire 5550 is moved from the first distance d1 to a second distance d2, the distal tip 5556 of the guide wire 5550 is also advanced into the bone tissue. In some embodiments, for example, the distal end portion 5554 (see e.g., FIG. 7) can include a threaded portion to allow the guide wire 5550 to be advanced into the bone tissue when the guide wire 5550 is rotated within the handle 5500, without striking the proximal end surface 5566 of the guide wire 5550 with a hammer.

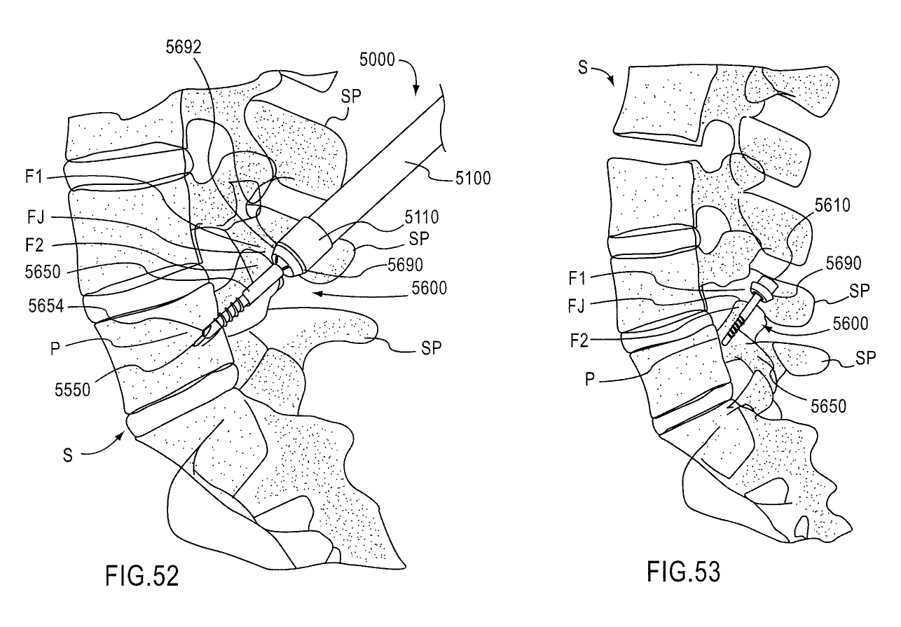

As shown in the lateral view depicted in FIG. 52, after the passageway is defined within the bone tissue, the bone screw 5650 is threaded into the passageway by rotating the first shaft 5100 and the second shaft 5200 together. Said another way, the bone screw 5650 is threaded into the passageway by placing the locking mechanism 5300 in the locked configuration and rotating the handle 5500. In this manner, the bone screw 5650 is threaded into the passageway without moving the nut 5610 relative to the bone screw 5650. The bone screw 5650 can be threaded into the bone tissue such that the distal end 5654 of the bone screw 5650 advances through the inferior facet F1 of the superior level, across the facet joint FJ, through the superior facet F2 of the inferior level, and into the pedicle P of the inferior level. Moreover, the bone screw 5650 can be threaded into the bone tissue such that the distal end surface 5692 of the washer 5690 is adjacent the inferior facet F1 of the superior level. In some embodiments, for example, the bone screw 5650 can be threaded into the bone tissue such that the distal end surface 5692 of the washer 5690 is in contact with the inferior facet F1 of the superior level. Although the bone screw 5650 is shown as being threaded into the passageway when the guide wire 5550 is within the passageway, in other embodiments, the guide wire 5550 can be removed from the insertion tool 5000 after the passageway is defined and before the bone screw 5650 is threaded into the passageway.

After the distal end 5654 of the bone screw 5650 is disposed within the pedicle P of the inferior level, the locking mechanism 5300 (not shown in FIGS. 48-54, see e.g., FIG. 31) can then be placed in the unlocked configuration, as described above. The nut 5610 can then be moved relative to the bone screw 5650 by rotating the first shaft 5100 about the second shaft 5200 (not shown in FIG. 51) Said another way, the nut 5610 can be tightened onto the bone screw 5650 without removing the insertion tool 5000 from the body. When the nut 5610 is being tightened, the washer 5690 can rotate relative to the bone screw 5650 along an axis normal to the longitudinal axis A_{L} of the medical device such that the distal end surface 5692 of the washer can be disposed flush against the surface of the bone. In this manner, the clamping load applied by tightening the nut 5610 can be substantially uniformly distributed along the surface of the bone.

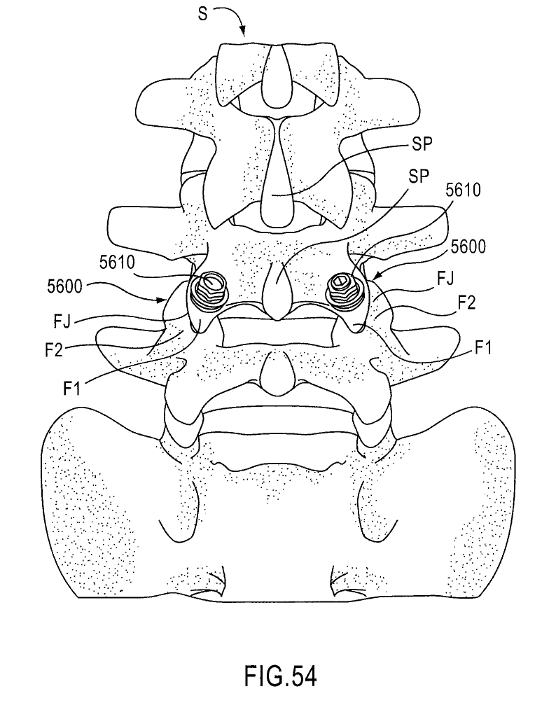

After the bone fixation device 5600 is inserted within the targeted bone tissue, the engagement portion 5110 of the first shaft 5100 can be decoupled from the nut 5610 by pulling the first shaft 5100 proximally. The insertion tool 5000 can then be removed from the body. FIGS. 53 and 54 show a lateral view and a posterior view, respectively of the bone fixation device 5600 after being inserted according to the procedures described above.

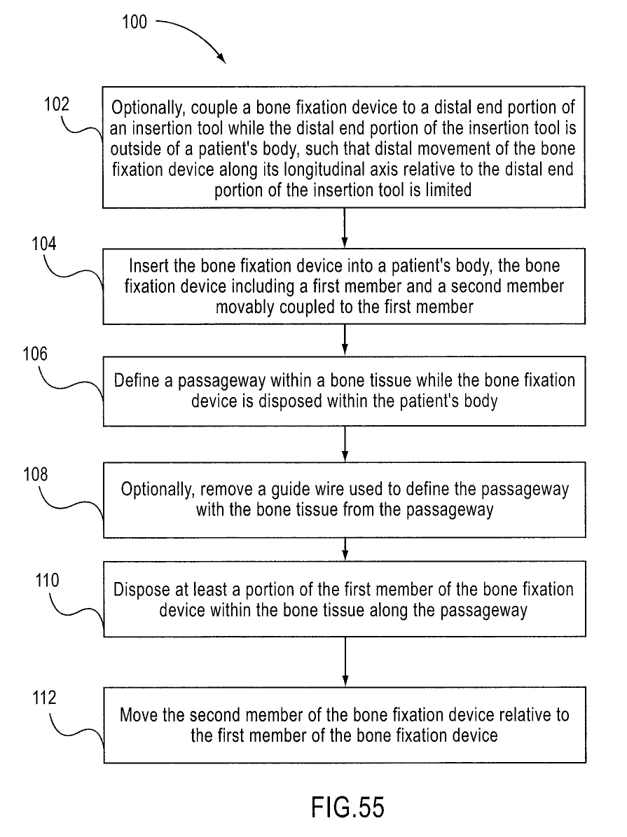

FIG. 55 is a flow chart illustrating a method 100 of inserting a bone fixation device into a body according to an embodiment of the invention. The illustrated method includes inserting a bone fixation device into a body, 104. The bone fixation device including a first member and a second member movably coupled to the first member. The bone fixation device can be any suitable bone fixation device, such as, for example, bone fixation device 5600 shown and described above with reference to FIGS. 37-47. In some embodiments, the first member of the bone fixation device can be a bone screw, such as, for example, bone screw 5650 shown and described above. In some embodiments, the second member of the bone fixation device can be a nut, such as, for example, nut 5610 shown and described above. In some embodiments, the bone fixation device can be inserted using the insertion tool. In some embodiments, the bone fixation device can be inserted percutaneously through an incision. For example, in some embodiments, the bone fixation device can be inserted in a minimally-invasive manner through an incision having a size less than 15 mm.

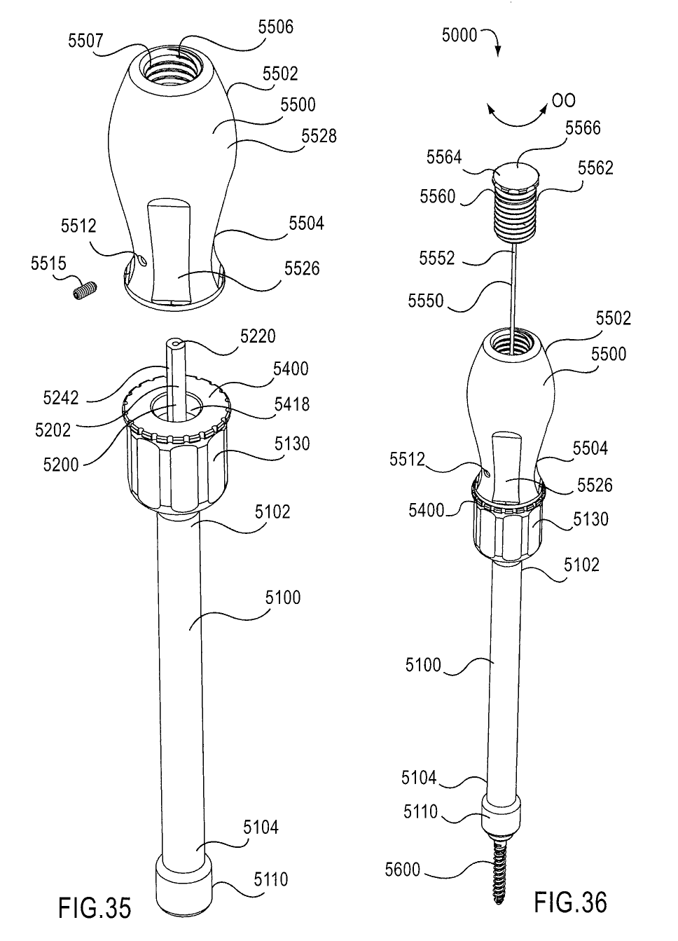

In some embodiments, the method optionally includes coupling the bone fixation device to a distal end portion of an insertion tool while the distal end portion of the insertion tool is outside of the patient's body, such that distal movement of the bone fixation device along its longitudinal axis relative to the distal end portion of the insertion tool is limited, 102. The insertion tool can be any suitable insertion tool, such as, for example, the insertion tool 5000 shown and described above with reference to FIGS. 7-36. In some embodiments, for example, the bone fixation device can be removably coupled to the distal end portion of the insertion tool by a retention member (see e.g., nut retention member 5160 described above with reference to FIGS. 22-24), a snap ring, a magnetic coupling, an adhesive coupling or the like.

A passageway is defined within a bone tissue while the bone fixation device is disposed within the patient's body, 106. In some embodiments, the passageway can be defined using the insertion tool. Moreover, in some embodiments, the passageway can be defined using the insertion tool without removing the distal end portion of the insertion tool from the body after the bone fixation device is inserted and before the passageway is defined. Similarly stated, in some embodiments, the distal end portion of the insertion tool can be coupled to the bone fixation device when the bone fixation device is inserted and the passageway can be defined using the insertion tool while the distal end portion of the insertion tool remains coupled to the bone fixation device.

In some embodiments, the passageway can be defined by advancing a first shaft of the insertion tool into the bone tissue. For example, in some embodiments, the passageway can be defined by advancing a guide wire, such as, for example, guide wire 5550 shown and described above with reference to FIG. 36, into the bone tissue while the bone fixation device is disposed within the patient's body. The guide wire can be advanced into the bone tissue by any suitable means, such as for example, by applying an axial force to the proximal end of the guide wire (e.g., striking the proximal end of the guide wire with a mallet), by rotating the guide wire relative to the insertion tool, or the like. In some embodiments, the method can optionally include removing the guide wire from the passageway before the bone fixation device is inserted into the passageway, 108.

At least a portion of the first member of the bone fixation device is disposed within the bone tissue along the passageway, 110. In some embodiments, the first member of the bone fixation device can be disposed within the bone tissue using an insertion tool that is also used to define the passageway. Similarly stated, in some embodiments, a single tool can be used to define the passageway and dispose the bone fixation device within the passageway. Said another way, in some embodiments, the passageway can be defined by an insertion tool and the bone fixation device can be disposed within the passageway using the insertion tool without the insertion tool being removed from the body. In some embodiments, the first member of the bone fixation device can be disposed within the bone tissue using an insertion tool similar to the insertion tool 5000 shown and described above. For example, in some embodiments, the first member of the bone fixation device can be threaded into the passageway by rotating a shaft of the insertion tool, as described above.

The second member of the bone fixation device is moved relative to the first member of the bone fixation device, 112. In this manner, the first member of the bone fixation device and the second member of the bone fixation device can cooperatively apply a clamping load to the bone tissue. In some embodiments, the second member of the bone fixation device can be moved relative to the first member of the bone fixation device using an insertion tool that is also used to define the passageway and/or to dispose the first member of the bone fixation device within the passageway. Similarly stated, in some embodiments, a single tool can be used to define the passageway, dispose the bone fixation device within the passageway and/or move the second member of the bone fixation device relative to the first member of the bone fixation device. In some embodiments, the second member of the bone fixation device can be moved relative to the first member of the bone fixation device using an insertion tool similar to the insertion tool 5000 shown and described above. For example, in some embodiments, the second member of the bone fixation can be moved axially relative to the first member of the bone fixation device by rotating a shaft of the insertion tool, as described above.

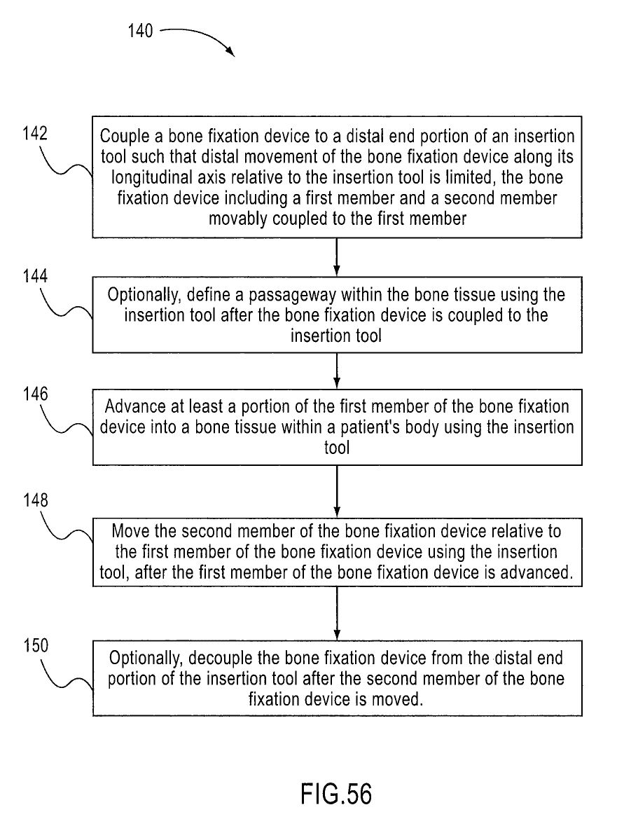

Although the method 100 is described above as including the operation of defining a passageway within a bone tissue, in other embodiments, a method can include inserting a bone fixation device without defining such a passageway. FIG. 56 is a flow chart illustrating a method 140 of inserting a bone fixation device into a body according to an embodiment of the invention. The illustrated method includes coupling a bone fixation device to a distal end portion of an insertion tool such that distal movement of the bone fixation device along its longitudinal axis relative to the insertion tool is limited, 142. The bone fixation device includes a first member and a second member movably coupled to the first member. The bone fixation device can be any suitable bone fixation device, such as, for example, bone fixation device 5600 shown and described above with reference to FIGS. 37-47. In some embodiments, the first member of the bone fixation device can be a bone screw, such as, for example, bone screw 5650 shown and described above. In some embodiments, the second member of the bone fixation device can be a nut, such as, for example, nut 5610 shown and described above. The insertion tool can be any suitable insertion tool, such as, for example, the insertion tool 5000 shown and described above with reference to FIGS. 7-36.

In some embodiments, the bone fixation device can be coupled to the distal end portion of an insertion tool by disposing a portion of the bone fixation device within a recess defined by the distal end portion of the insertion tool such that a retention member of the insertion tool is removably disposed within a groove defined by the proximal end portion of the bone fixation device. In other embodiments, the bone fixation device can be coupled to the distal end portion of an insertion tool by threadedly coupling the insertion tool to the first member of the bone fixation device. For example, in some embodiments, the bone fixation device can be coupled to the distal end portion of an insertion tool by threadedly coupling a shaft of the insertion tool within a recess defined by the first member of the bone fixation device, as described in more detail below. Although the bone fixation device can be coupled to the distal end portion of an insertion tool by a mechanical coupling, in other embodiments, the bone fixation device can be coupled to the distal end portion of an insertion tool using a magnetic coupling, an adhesive coupling, an electronic coupling or the like.

In some embodiments, the method can optionally include defining a passageway within the bone tissue using the insertion tool after the bone fixation device is coupled to the insertion tool, 144. For example, in some embodiments, the passageway can be defined by advancing a guide wire (e.g., guide wire 5550) through the bone fixation device and into the bone tissue while the bone fixation device is coupled to the insertion tool and/or while the bone fixation device is disposed within a body. The guide wire can be advanced into the bone tissue by any suitable means, as described herein.

At least a portion of the first member of the bone fixation device is advanced into a bone tissue within a body using the insertion tool, 146. In some embodiments, the bone fixation device can be advanced by rotating a first shaft of the insertion tool such that at least the first member of the bone fixation device is threaded into the bone tissue. Moreover, in some embodiments that include defining a passageway within the bone tissue, the passageway can be defined by the insertion tool and at least a portion of the bone fixation device can be disposed within the passageway using the insertion tool without the insertion tool being removed from the body.

The second member of the bone fixation device is then moved relative to the first member of the bone fixation device using the insertion tool, 148. In this manner, a single tool can be used to advance the bone fixation device into the bone tissue and to move the second member of the bone fixation device relative to the first member of the bone fixation device. In some embodiments, for example, the second member can be moved by rotating a second shaft of the insertion tool relative to a first shaft of the insertion tool. In some embodiments, the method can optionally include decoupling the bone fixation device from the distal end portion of the insertion tool after the second member of the bone fixation device is moved, 150.

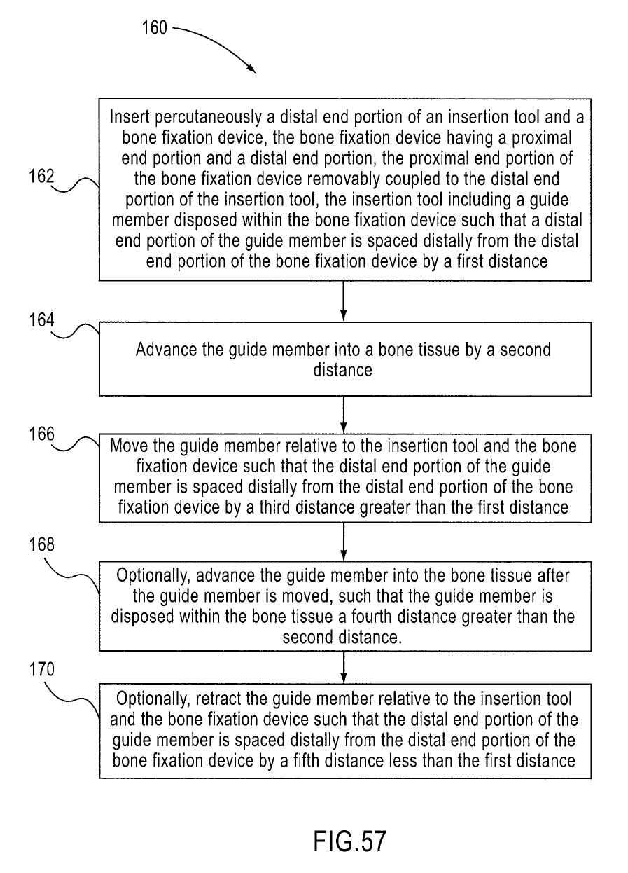

Although the method 100 is described above as including the operation of defining a passageway within a bone tissue, in some embodiments, a method can include iteratively defining such a passageway. FIG. 57 is a flow chart illustrating a method 160 of inserting a bone fixation device into a body according to an embodiment of the invention. The method includes inserting percutaneously a distal end portion of an insertion tool and a bone fixation device, 162. The bone fixation device has a proximal end portion and a distal end portion. The proximal end portion of the bone fixation device is removably coupled to the distal end portion of the insertion tool. The insertion tool includes a guide member disposed within the bone fixation device such that a distal end portion of the guide member is spaced distally from the distal end portion of the bone fixation device by a first distance. The bone fixation device can be any suitable bone fixation device, such as, for example, bone fixation device 5600 shown and described above with reference to FIGS. 37-47. The insertion tool can be any suitable insertion tool, such as, for example, the insertion tool 5000 shown and described above with reference to FIGS. 7-36.

The guide member is advanced into a bone tissue by a second distance, 164. The guide member can be any suitable member configured to guide the insertion of the insertion tool and/or the bone fixation device into the bone tissue. In some embodiments, for example, the guide member can be a guide wire similar to the guide wire 5550 shown and described above with reference to FIG. 36. The guide member can be advanced into the bone tissue by any suitable means, such as for example, by applying an axial force to the proximal end of the guide wire (e.g., striking the proximal end of the guide wire with a mallet), by rotating the guide wire relative to the insertion tool, or the like. In some embodiments, the second distance can be substantially equal to the first distance. Said another way, in some embodiments, the guide member can be advanced into the bone tissue such that the distal end portion of the bone fixation device is disposed against the surface of the bone tissue (e.g., the distal end portion of the bone fixation device is flush against the surface of the bone tissue). In other embodiments, however, the second distance can be less than or greater than the first distance.

The guide member is then moved relative to the insertion tool and the bone fixation device such that the distal end portion of the guide member is spaced distally from the distal end portion of the bone fixation device by a third distance greater than the first distance, 166. Said another way, after the guide member is advanced into the bone tissue, the position of the guide member relative to the bone fixation device is moved such that the distal end portion of the guide member extends beyond the distal end portion of the bone fixation device by a third distance greater than the first distance. Said yet another way, after the guide member is advanced into the bone tissue, the guide member is moved axially relative to the bone fixation device in the distal direction such that the distal end portion of the guide member extends beyond the distal end portion of the bone fixation device by a third distance greater than the first distance. The guide member can be moved relative to the insertion tool and the bone fixation device in any suitable manner, as described herein. For example, in some embodiments, the guide member can be moved by rotating a threaded portion of the guide member within a corresponding threaded portion of the insertion tool. In this manner, the guide member moves both rotationally and axially relative to the insertion tool and the bone fixation device. In some embodiments, the guide member can be moved relative to the insertion tool and the bone fixation device through a set of discrete increments. Said another way, in some embodiments, the guide member can be moved relative to the insertion tool and the bone fixation device in an incremental and/or controlled manner (e.g., using a ratchet mechanism).

In some embodiments, the method can optionally include advancing the guide member into the bone tissue after the guide member is moved, such that the guide member is disposed within the bone tissue a fourth distance greater than the second distance, 168. Said another way, in some embodiments, the method can optionally include advancing the guide member into the bone tissue a second time after the guide member is moved. In this manner, the guide member can be incrementally advanced into the bone tissue. In some embodiments, the guide member can be incrementally advanced into the bone tissue without removing the insertion tool and/or the bone fixation device from the body, as shown and described above with reference to FIGS. 48-54.

In some embodiments, the method can optionally include retracting the guide member relative to the insertion tool and the bone fixation device such that the distal end portion of the guide member is spaced distally from the distal end portion of the bone fixation device by a fifth distance less than the first distance, 170. Said another way, in some embodiments, the method can optionally include moving the guide member axially relative to the bone fixation device in the proximal direction such that the distal end portion of the guide member extends beyond the distal end portion of the bone fixation device by a fifth distance less than the first distance. In some embodiments, the guide member can be moved in the proximal direction until the distal end portion of the guide member is disposed proximally from the distal end portion of the bone fixation device. In some embodiments, the guide member can be moved in the proximal direction until the distal end portion of the guide member is removed from the bone fixation device and/or the insertion tool.

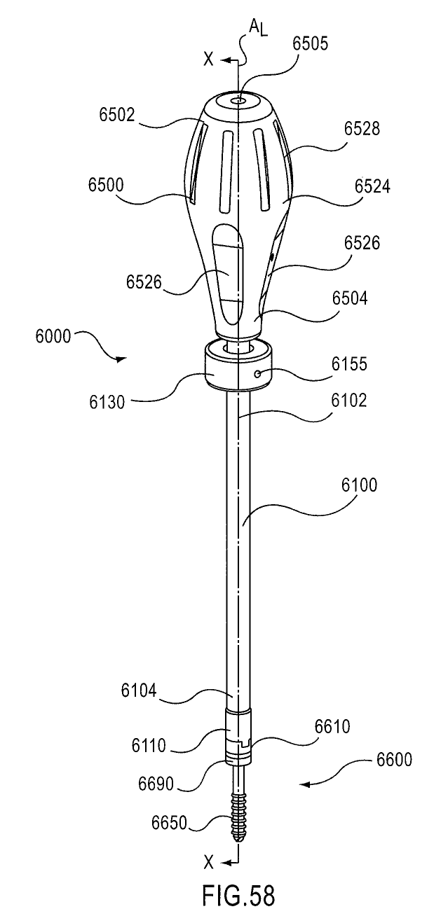

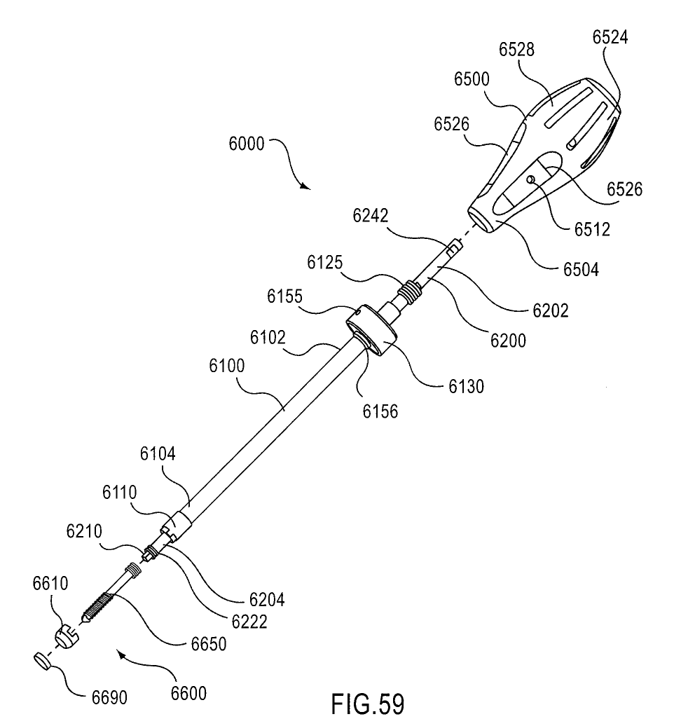

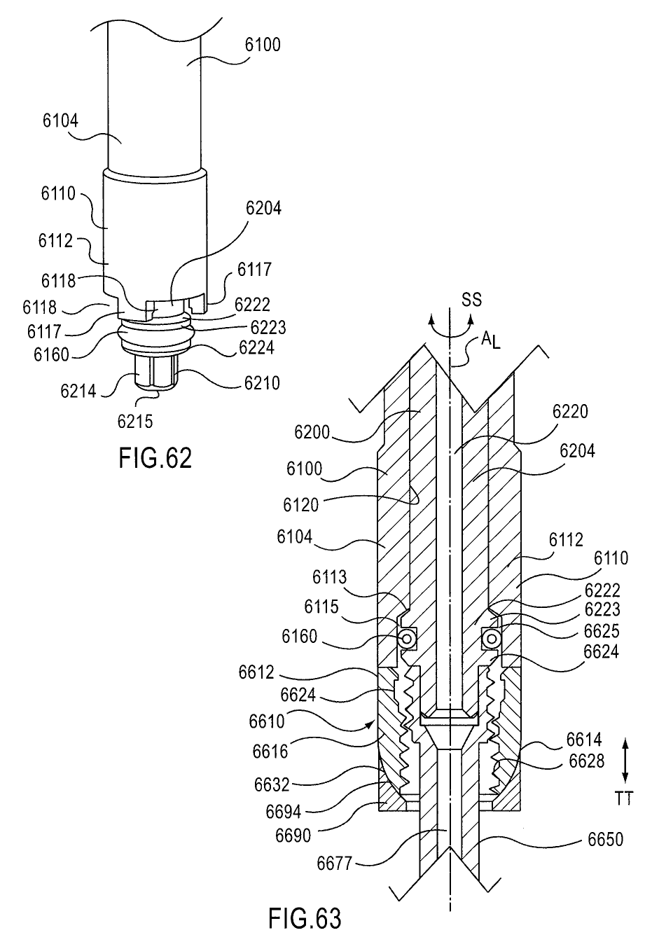

Although the insertion tool 5100 is shown and described above as including a nut engagement portion 5110 on the first shaft 5100 (i.e., the outer shaft) configured to selectively retain the nut 5610, in other embodiments, an insertion tool can retain a nut, a screw and/or any portion of a bone fixation device in any suitable manner. For example, in some embodiments, an insertion tool can selectively retain a bone fixation device via a nut engagement portion disposed on a second shaft (i.e., the inner shaft). One such embodiment is shown in FIGS. 58-66, which show an insertion tool 6000 and a bone fixation device 6600 according to an embodiment of the invention. The insertion tool 6000 includes a first shaft 6100, a second shaft 6200 (see FIGS. 59-61) and a handle 6500. Unlike the insertion tool 5000 shown and described above, the insertion tool 6000 does not include a locking mechanism or an actuator. The bone fixation device 6600 includes a nut 6610, a bone screw 6650, and a washer 6690.

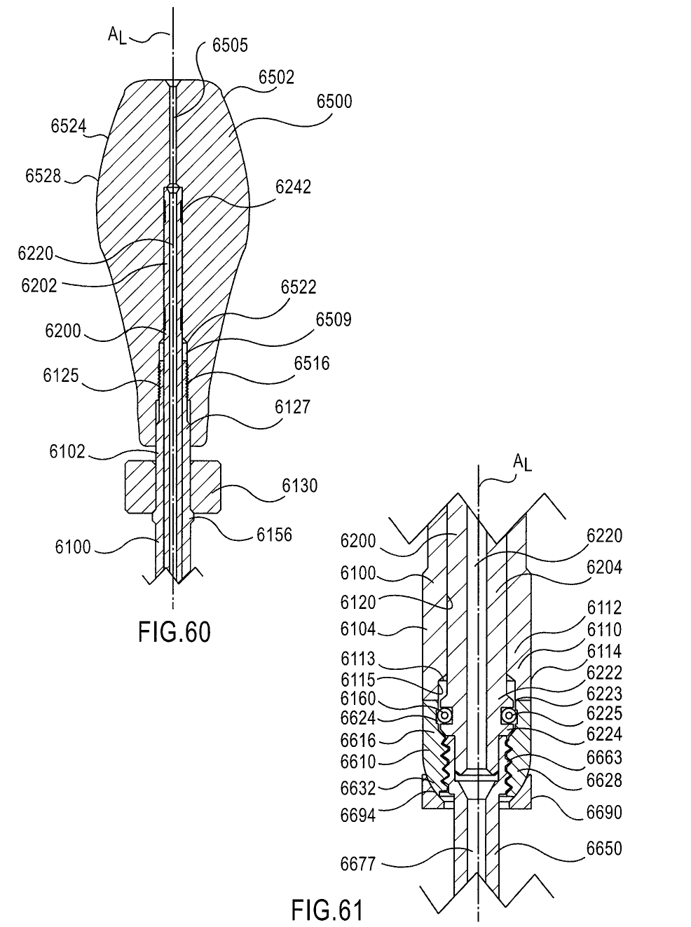

The first shaft 6100 includes a proximal end portion 6102 and a distal end portion 6104, and defines a lumen 6120 therethrough. As shown in FIGS. 60 and 61, the lumen 6120 defines a longitudinal axis A_{L}. As shown in FIGS. 59 and 60, the proximal end portion 6102 of the first shaft 6100 includes an actuator 6130, a first shoulder 6156, a second shoulder 6127, and a threaded portion 6125. The actuator 6130 is disposed about the outer surface of the first shaft 6100 such that a portion of the actuator 6130 engages the first shoulder 6156 defined by the first shaft 6100. The actuator 6130 is coupled to the outer surface of the first shaft 6100 using a set screw 6155. In this manner, the actuator 6130 can be used to rotate the first shaft 6100 of the insertion tool 6000 about the longitudinal axis A_{L}. Although the outer surface of the actuator 6130 is shown as being relatively smooth, in other embodiments, the outer surface of the actuator 6130 can include any suitable topographical features to aid in grasping and rotating the actuator 6130 and therefore the first shaft 6100. For example, in some embodiments, the outer surface of the actuator 6130 can include multiple alternating protrusions and recesses, a knurled portion or the like.

The threaded portion 6125 of the first shaft 6100 includes male threads on a portion of the outer surface of the proximal end portion 6102 of the first shaft 6100. As shown in FIG. 60, at least a portion of the proximal end portion 6102 of the first shaft 6100 is disposed within an opening 6509 defined by the handle 6500 such that the threaded portion 6125 engages a corresponding threaded portion 6516 of the handle 6500. In this manner, when the first shaft 6100 rotates about the longitudinal axis A_{L} relative to the handle 6500 (as shown by the arrow SS in FIG. 63), the first shaft 6100 moves axially relative to the handle 6500 and/or the second shaft 6200 (as shown by the arrow TT in FIG. 63). The amount of axial movement of the first shaft 6100 relative to the handle 6500 and/or the second shaft 6200 is associated with the thread pitch of the threaded portion 6125 first shaft 6100 and/or the threaded portion 6516 of the handle 6500. In this manner, the first shaft 6100 can be moved axially relative to the handle 6500 and/or the second shaft 6200 in a controlled and/or incremental fashion. Additionally, the second shoulder 6127 of the first shaft 6100 is configured to contact a portion of the threaded portion 6516 of the handle 6500 to limit the axial motion of the first shaft 6100 relative to the handle 6500 and/or the second shaft 6200 in the proximal direction.

As shown in FIGS. 61-63, the distal end portion 6104 of the first shaft 6100 includes a nut engagement portion 6110. The nut engagement portion 6110 includes a side wall 6112 having an outer surface 6114 and an inner surface 6115. The inner surface 6115 of the nut engagement portion 6110 defines a shoulder 6113 configured to contact a proximal protrusion 6223 of the engagement portion 6210 of the second shaft 6200. Said another way, the shoulder 6113 of the first shaft 6100 and the proximal protrusion 6223 of the second shaft 6200 are configured to cooperatively limit the axial motion of the second shaft 6200 within the first shaft 6100 in the proximal direction. Said another way, the shoulder 6113 of the first shaft 6100 and the proximal protrusion 6223 of the second shaft 6200 are configured to cooperatively limit the axial motion of the first shaft 6100 about the second shaft 6200 in the distal direction. Accordingly, the second shoulder 6127 of the first shaft 6100, the threaded portion 6516 of the handle 6500, the shoulder 6113 of the first shaft 6100, and the proximal protrusion 6223 of the second shaft 6200 are configured to cooperatively limit the range of axial motion of the first shaft 6100 with respect to the second shaft 6200.

The distal end of the nut engagement portion 6110 of the first shaft 6100 includes a series of alternating protrusions 6117 and openings 6118 configured to matingly receive the nut 6610 of the bone fixation device 6600. Said another way, the alternating protrusions 6117 and openings 6118 of the first shaft 6100 correspond to the alternating protrusions 6621 and openings 6622 of the nut 6610. In this manner, the nut 6610 can be engaged with the nut engagement portion 6110 of the first shaft 6100 such that rotation of the first shaft 6100 about the longitudinal axis A_{L}, as shown by the arrow SS in FIG. 63, results in rotation of the nut 6610.

As shown in FIGS. 61 and 63, the outer surface 6114 of the nut engagement portion 6110 has an outer diameter that is substantially equal to the outer diameter of the nut 6610. In this manner, the overall profile of the medical device 6000 can be reduced, thereby allowing the insertion of the bone fixation device 6600 via small incisions.

As best shown in FIGS. 59-61, the second shaft 6200 includes a proximal end portion 6202, a distal end portion 6204, and defines a lumen 6220 therethrough. As shown in FIGS. 59 and 60, the proximal end portion 6202 of the second shaft 6200 is configured to be received within a distal opening 6509 defined by the handle 6500. More particularly, the proximal end portion 6202 of the second shaft 6200 includes a series of flatted surfaces 6242 that provide an engagement surface for the set screw 6512 of the handle 6500. In this manner, second shaft 6200 can be coupled within the handle 6500 such that the handle 6500 can be used to rotate the second shaft 6200 of the insertion tool 6000 about the longitudinal axis A_{L}. Moreover, in this manner, the second shaft 6200 can be coupled within the handle 6500 such that axial movement of the handle 6500 results in axial movement of the second shaft 6200. Said another way, the second shaft 6200 can be coupled within the handle 6500 such that axial movement of the handle 6500 relative to the first shaft 6100 results in an equivalent axial movement of the second shaft 6200 relative to the first shaft 6100.

As shown in FIGS. 61-63, the distal end portion 6204 of the second shaft 6200 includes an engagement portion 6210. The engagement portion 6210 includes a first surface 6214, a second surface 6222, and a distal end surface 6215. The first surface 6214 of the engagement portion 6210 includes a set of hexagonal shaped portions corresponding to the hexagonal shaped recess 6660 defined within the engagement portion 6656 of the bone screw 6650. In this manner, the engagement portion 6210 of the second shaft 6200 can be received within the engagement portion 6656 of the bone screw 6650 such that rotation of the second shaft 6200 about the longitudinal axis A_{L} results in rotation of the bone screw 6650.

The second surface 6222 of the engagement portion 6210 is disposed proximally from the first surface 6214 of the engagement portion 6210 and includes a proximal protrusion 6223, a distal protrusion 6224 and defines a groove 6225 therebetween (best shown in FIG. 63). The groove 6225 receives a nut retention member 6160. As described above, the nut retention member 6160 can be any suitable member (e.g., a coil spring, a snap ring or the like) configured to received within a groove 6624 of the nut 6610. In this manner, the engagement portion 6210 of the second shaft 6200 and the nut retention member 6160 can selectively retain the nut 6610 to limit movement of the nut 6610 relative to the second shaft 6200 along the longitudinal axis A_{L}.

The proximal protrusion 6223 of the second shaft has an outer diameter that is greater than an outer diameter of at least a portion of the lumen 6120 of the first shaft 6100. Accordingly, as described above, the proximal protrusion 6223 is configured to contact the shoulder 6113 of the nut engagement portion 6110 of the first shaft to limit the axial motion of the second shaft 6200 within the first shaft 6100 in the proximal direction.

As shown in FIGS. 59, 61 and 63, at least a portion of the second shaft 6200 is disposed within the lumen 6120 of the first shaft 6100 such that the first shaft 6100 and the second shaft 6200 are coaxial about the longitudinal axis A_{L}. The portion of the second shaft 6200 is disposed within the lumen 6120 of the first shaft 6100 such that the second shaft 6200 can rotate about the longitudinal axis A_{L} relative to the first shaft 6100. Moreover, the portion of the second shaft 6200 is disposed within the lumen 6120 of the first shaft 6100 such that the second shaft 6200 can move axially (i.e., along the longitudinal axis A_{L}, as shown by the arrow TT in FIG. 63) relative to the first shaft 6100. As described above, the proximal protrusion 6223 of the second shaft has an outer diameter that is greater than an outer diameter of at least a portion of the lumen 6120 of the first shaft 6100. Accordingly, as shown in FIG. 63, the proximal protrusion 6223 is configured to contact the shoulder 6113 of the nut engagement portion 6110 of the first shaft to limit the axial motion of the second shaft 6200 within the first shaft 6100 in the proximal direction.

As shown in FIGS. 58-60, the handle 6500 includes a proximal portion 6502 and a distal portion 6504. The proximal portion 6502 of the handle 6500 defines a lumen 6505 configured to receive a guide member (not shown), such as for example, a guide wire, a K-wire or the like. As shown in FIG. 60 and described above, the handle 6500 is configured to be coupled to and receive a portion of the second shaft 6200 such that the lumen 6505 of the handle 6500 is substantially coaxial with the lumen 6220 of the second shaft 6200. In this manner, a guide wire (not shown) can be disposed within and/or through the lumen 6505 and the lumen 6220.

The distal portion 6504 of the handle 6500 defines an opening 6509 that is coaxial with and in fluid communication with the lumen 6505. As described above, the opening 6509 is configured to receive a portion of the first shaft 6100 and a portion of the second shaft 6200. Moreover, as described above, the surface defining the opening 6509 includes a threaded portion 6516 configured to engage the threaded portion 6125 of the first shaft 6100. Said another way, the surface defining the opening 6509 defines a female threaded portion 6516 configured to engage the corresponding male threaded portion 6125 of the first shaft 6100. In this manner, when the first shaft 6100 rotates about the longitudinal axis A_{L} relative to the handle 6500 and/or the second shaft 6200, the first shaft 6100 moves axially relative to the handle 6500 and/or the second shaft 6200. Additionally, the surface defining the opening 6509 defines a shoulder 6522 configured to contact the proximal end of the first shaft 6100 to limit the axial motion of the first shaft 6100 relative to the handle 6500 and/or the second shaft 6200 in the proximal direction.

The handle 6500 includes a transverse lumen (not shown) that is substantially normal to the longitudinal axis A_{L}. The transverse lumen intersects the opening 6509 adjacent one of the flatted surfaces 6242 of the second shaft 6200, and is configured to threadedly receive a set screw 6512. In this manner, the second shaft 6200 can be coupled within the handle 6500 by the set screw 6512 such that the handle 6500 can be used to rotate the second shaft 6200 and/or the first shaft 6100 about the longitudinal axis A_{L}. The handle 6500 includes an outer surface 6524, which includes a bulb-shaped portion 6528 and multiple flats 6526. Accordingly, the outer surface 6524 of the handle 6500 is configured to be grasped and/or manipulated by the user, for example, to rotate the first shaft 6100 and/or the second shaft 6200 about the longitudinal axis A_{L}.

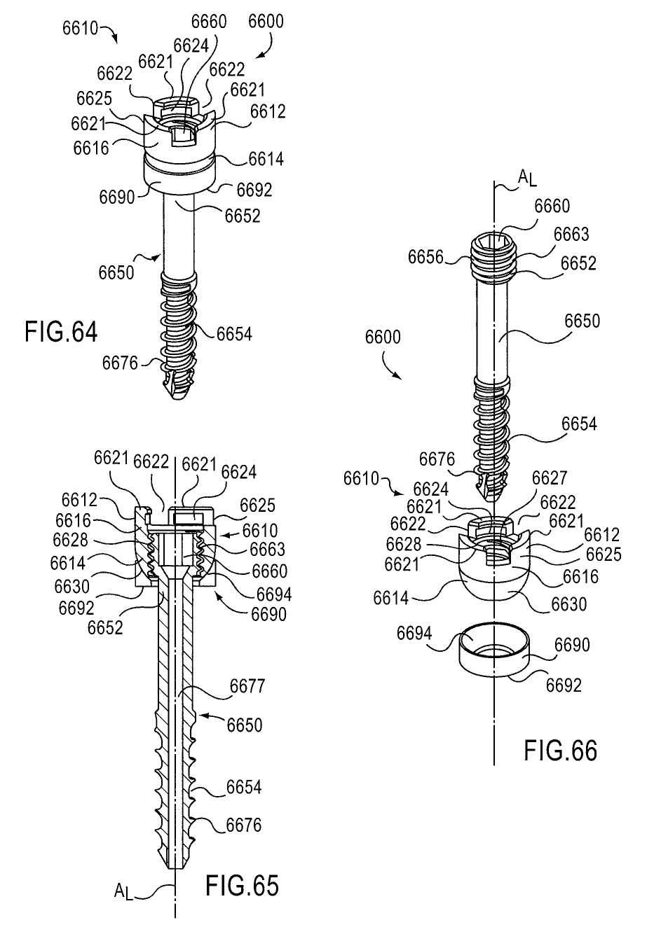

As shown in FIGS. 64-66, the bone fixation device 6600 includes a nut 6610, a bone screw 6650, and a washer 6690. The bone screw 6650 includes a proximal end portion 6652 and a distal end portion 6654. The bone screw 6650 defines a lumen 6677 that is coaxial with the longitudinal axis A_{L} when the bone fixation device 6600 is coupled to the insertion tool 6000. In this manner, a guide member (not shown) can be disposed within the lumen 6220 of the second shaft and the lumen 6677 of the bone screw 6650, as described herein.

Similar to the bone screw 5650 described above, the distal end portion 6654 of the bone screw 6650 includes a self-tapping tip and threaded portion 6676. The proximal end portion 6652 of the bone screw includes an engagement portion 6656 defining a hexagonal shaped recess 6660 corresponding to the hexagonal shaped portions of the engagement portion 6210 of the second shaft 6200. In this manner, the engagement portion 6656 of the bone screw 6650 can receive a portion of the engagement portion 6210 of the second shaft 6200 such that rotation of the second shaft 6200 about the longitudinal axis A_{L} results in rotation of the bone screw 6650.

The engagement portion 6656 of the bone screw 6650 also includes a threaded portion 6663. The threaded portion 6663 includes male threads that correspond to the threaded portion 6628 of the nut 6610. The thread pitch of the threaded portion 6663 of the bone screw 6650 and the thread pitch of the threaded portion 6628 of the nut 6610 is substantially the same as the thread pitch of the threaded portion 6125 of the first shaft 6100 and the threaded portion 6516 of the handle. Accordingly, when bone fixation device 6600 is coupled to the insertion tool 6000 and when the second shaft 6200 is rotated within the first shaft 6100, the distance through which the second shaft 6200 moves axially relative to the first shaft 6100 is substantially the same as the distance through which the nut 6610 moves axially relative to the bone screw 6650.

As shown in FIGS. 64-66, the nut 6610 includes a proximal end portion 6612 and a distal end portion 6614, and defines a lumen 6627 therethrough. The nut 6610 is configured to be threadedly coupled to the bone screw 6650 such that the lumen 6627 of the nut 6610 is substantially concentric with the longitudinal axis A_{L} of the bone screw 6650.

The proximal end portion 6612 of the nut 6610 includes an engagement portion 6625 that includes a side wall 6616. The side wall 6616 includes a series of alternating protrusions 6621 that define corresponding openings 6622 therebetween. As described above, the alternating protrusions 6621 and openings 6622 of the nut 6610 correspond to the alternating protrusions 6117 and openings 6118 of the first shaft 6100. In this manner, the nut engagement portion 6110 of the first shaft 6100 can engage the nut 6610 such that rotation of the first shaft 6100 about the longitudinal axis A_{L}, as shown by the arrow SS in FIG. 63, results in rotation of the nut 6610. Said another way, the engagement portion 6625 of the nut 6610 can be engaged with the nut engagement portion 6110 of the first shaft 6100 such that rotational movement of the nut 6610 relative to the first shaft 6100 is limited.

The inner surface of the side wall 6616 defines a groove 6624 at a position along the longitudinal axis A_{L}. As shown in FIG. 61, the groove 6624 is configured to receive a portion of a nut retention member 6160. As discussed above, a portion of the nut retention member 6160 is also disposed within the groove 6225 defined by the of the engagement portion 6210 of the second shaft 6200. In this manner, the nut retention member 6160 can selectively couple the nut 6610 to the engagement portion 6210 of the second shaft 6200.

The nut 6610 includes a threaded portion 6628 within the lumen 6627. Said another way, the nut 6610 defines a female threaded portion 6628. As described above, the threaded portion 6628 of the nut 6610 corresponds to the threaded portion 6663 of the bone screw 6650, the threaded portion 6126 of the first shaft 6100 and/or the threaded portion 6516 of the handle 6500. In some embodiments, the distal-most thread of the threaded portion 6628 can be crimped such that the axial motion of the nut 6610 relative to the bone screw 6650 in the proximal direction is limited. Said another way, in some embodiments, the distal-most thread of the threaded portion 6628 can have an inner diameter that is less than the outer diameter of the threaded portion 6663 of the bone screw 6650.

The outer surface the distal end portion 6614 of the nut 6610 includes a curved surface 6630 configured to contact the washer 6690. More particularly, as described above, the curved surface 6630 of the nut 6610 corresponds to the curved surface 6694 of the washer 6690, such that a portion of washer 6690 can be matingly disposed about the curved surface 6630 of the nut 6610. In this manner, when the nut 6610 is tightened on the bone screw 6650, the clamping load is transferred in a substantially uniform and/or spatially distributed fashion to the washer 6690. Moreover, as described above, this arrangement allows the washer 6690 to rotate relative to the nut 6610 and/or the bone screw 6650 about an axis substantially normal to the longitudinal axis A_{L} of the nut 6610.

The washer 6690 includes a distal end surface 6692 and a curved surface 6694. As described above, the distal end surface 6692 is configured to engage the targeted bone tissue when the nut 6610 is tightened on the bone screw 6650. The curved surface 6694 is configured to contact the curved surface of the nut 6610, as described above.

As described above, the first shaft 6100 can be rotated about the second shaft 6200 to move the insertion tool 6000 and the bone fixation device 6600 between a first configuration (FIG. 61) and a second configuration (FIG. 63). In the first configuration, the engagement portion 6210 of the second shaft 6200 is disposed within the nut 6610 such that the nut retention member 6160 is disposed within the groove 6624 of the nut. Accordingly, the nut 6610 is selectively coupled to the second shaft 6200 to limit movement of the nut 6610 relative to the second shaft 6200 along the longitudinal axis A_{L}. Similarly stated, when the insertion tool 6000 and the bone fixation device 6600 are in the first configuration, the bone fixation device 6600 is selectively coupled to the insertion tool 6000. Moreover, when the insertion tool 6000 and the bone fixation device 6600 are in the first configuration, the engagement portion 6210 of the second shaft 6200 is received within the engagement portion 6656 of the bone screw 6650. Similarly stated, when the insertion tool 6000 and the bone fixation device 6600 are in the first configuration, the hexagonal shaped portions of the engagement portion 6210 are matingly received within the hexagonal shaped recess 6660 of the engagement portion 6656 of the bone screw 6650.

When the insertion tool 6000 and the bone fixation device 6600 are in the first configuration, the distal end portion of the insertion tool 6000 and the bone fixation device 6600 can be inserted into the body and positioned adjacent a target bone tissue, as described above. Although the insertion tool 6000 is not shown and described as including a guide member, in other embodiments, the insertion tool can include a guide member, similar to the guide wire 5550, to locate the target bone tissue and/or define a passageway within the bone tissue as described above. When the distal end portion 6654 of the bone screw 6650 is disposed against the bone tissue and with the insertion tool 6000 and the bone fixation device 6600 in the first configuration, the bone screw 6650 can be threaded into the bone tissue by rotating the second shaft 6200.

Although the insertion tool 6000 is devoid of a locking mechanism similar to locking mechanism 5300 shown and described above, when the insertion tool 6000 and the bone fixation device 6600 are in the first configuration, the force imparted by the nut retention member 6160 within the groove 6624 can selectively limit the rotational motion of the nut 6610 relative to the second shaft 6200. Similarly stated, the frictional force caused by the compression of the nut retention member 6160 within the groove 6624 opposes the rotational motion of the nut 6610 relative to the second shaft 6200. Accordingly, when the second shaft 6200 is rotated about the longitudinal axis A_{L}, the nut 6610 rotates with the second shaft 6200 until an external force opposing the rotation of the nut 6610 exceeds the frictional force caused by the compression of the nut retention member 6624 within the groove 6624. Because the first shaft 6100 is engaged with the nut 6610, the rotation of the nut 6610 with the second shaft 6200 results in the first shaft 6100 rotating with the second shaft 6200. In this manner, the nut retention member 6160, the groove 6624 and/or the engagement portion 6210 of the second shaft 6200 selectively lock the first shaft 6100 to the second shaft 6200. Said another way, when the insertion tool 6000 and the bone fixation device 6600 are in the first configuration, rotation of the second shaft 6200 relative to the first shaft 6100 is prevented until a force causing rotation of the second shaft 6200 relative to the first shaft 6100 exceeds a predefined value.

The predefined value (i.e., the threshold of the friction force caused by the compression of the nut retention member 6160 within the groove 6624) is associated with the characteristics of the retention member 6160, the groove 6624 of the nut 6610 and/or the groove 6225 of the second shaft 6200. For example, in some embodiments, the nut retention member 6160 can be a canted coiled spring. The threshold of the friction force in such embodiments can be changed by changing the spring characteristics of the nut retention member 6160, the outer diameter of the nut retention member 6160 and/or the material from which the nut retention member 6160 is constructed.

When the bone screw 6650 is threaded into the bone tissue, the insertion tool 6000 and the bone fixation device 6600 can be moved from the first configuration to the second configuration by rotating the first shaft 6100 about the second shaft 6200, as shown by the arrow SS in FIG. 63. Said another way, the insertion tool 6000 and the bone fixation device 6600 can be moved from the first configuration to the second configuration by rotating the nut 6610 relative to the bone screw 6650 (e.g., by “tightening” the nut 6610). The insertion tool 6000 and the bone fixation device 6600 can be moved from the first configuration to the second configuration by applying a rotational force to the first shaft 6100 that exceeds the friction force caused by the compression of the nut retention member 6160 within the groove 6624. Such a force can be applied, for example, by maintaining the rotational position of the handle 6500 and applying a rotational force to the first shaft 6100 via the actuator 6130.

When the first shaft 6100 is rotated about the second shaft 6200, the first shaft 6100 moves distally along the longitudinal axis A_{L} relative to the second shaft 6200, as shown by the arrow TT in FIG. 63. The amount of axial movement of the first shaft 6100 relative to the second shaft 6200 is associated with the thread pitch of the threaded portion 6125 first shaft 6100 and the threaded portion 6516 of the handle 6500. In this manner, the first shaft 6100 can be moved axially relative to the second shaft 6200 in a controlled and/or incremental fashion. Moreover, the thread pitch of the threaded portion 6663 of the bone screw 6650 and/or the thread pitch of the threaded portion 6628 of the nut 6610 is substantially the same as the thread pitch of the threaded portions 6125 and the threaded portion 6516. Accordingly, when the first shaft 6100 is rotated about the second shaft 6200, the distance through which the first shaft 6100 moves axially relative to the second shaft 6200 is substantially the same as the distance through which the nut 6610 moves axially relative to the bone screw 6650.

As shown in FIG. 63, when the first shaft 6100 is moved distally along the longitudinal axis A_{L} relative to the second shaft 6200 (i.e., by rotating the first shaft 6100 about the second shaft 6200), the nut 6610 moves distally relative to the bone screw 6650. Accordingly, the groove 6624 of the nut 6610 moves out of axial alignment with the nut retention member 6160 and/or the engagement portion 6210 of the second shaft 6200, thereby causing the nut retention member 6160 be displaced from the groove 6624 of the nut 6610. In this manner, when the insertion tool 6000 and the bone fixation device 6600 are in the second configuration, the nut 6610 is not coupled to the second shaft 6200.

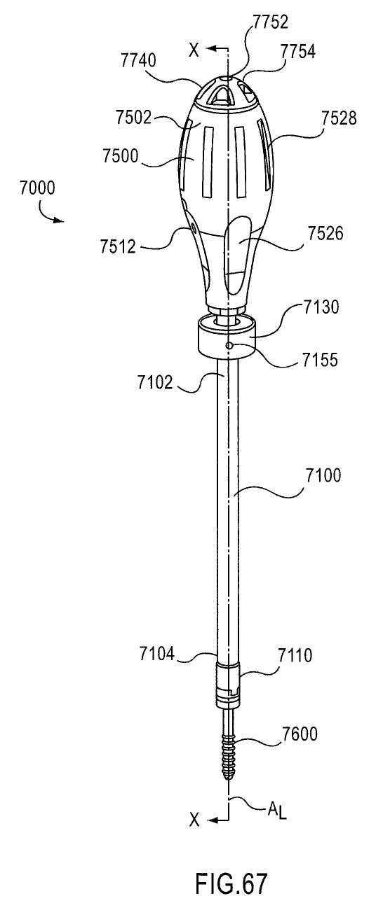

Although the insertion tools 5000 and 6000 are shown and described above as being removably coupleable to the bone fixation device 6600 via the nut retention member 5160 and 6160, respectively, in other embodiments, an insertion tool can be coupleable to a bone fixation device by any suitable means. For example, in some embodiments, an insertion tool can be removably coupleable to a bone fixation device via a magnetic coupling. In other embodiments, an insertion tool can be removably coupleable to a bone fixation device via a threaded coupling. One such embodiment is shown in FIGS. 67-76, which show an insertion tool 7000 according to an embodiment of the invention as used with a bone fixation device 7600 shown and described above. The bone fixation 7600 device includes a bone screw 7650, a nut 6610 (see e.g., FIGS. 64-66) and a washer 6690 (see e.g., FIGS. 64-66). The bone screw 7650 is similar to the bone screw 6650 shown and described above, except, as shown in FIG. 70, the bone screw 7650 includes a female threaded portion 7667 within the engagement portion 7656 of the bone screw 7650 adjacent the hexagonal shaped recess 7660. Because the bone fixation device 7600 is similar in many respects to the bone fixation device 6600, the bone fixation device 7600 is not discussed in great detail below.

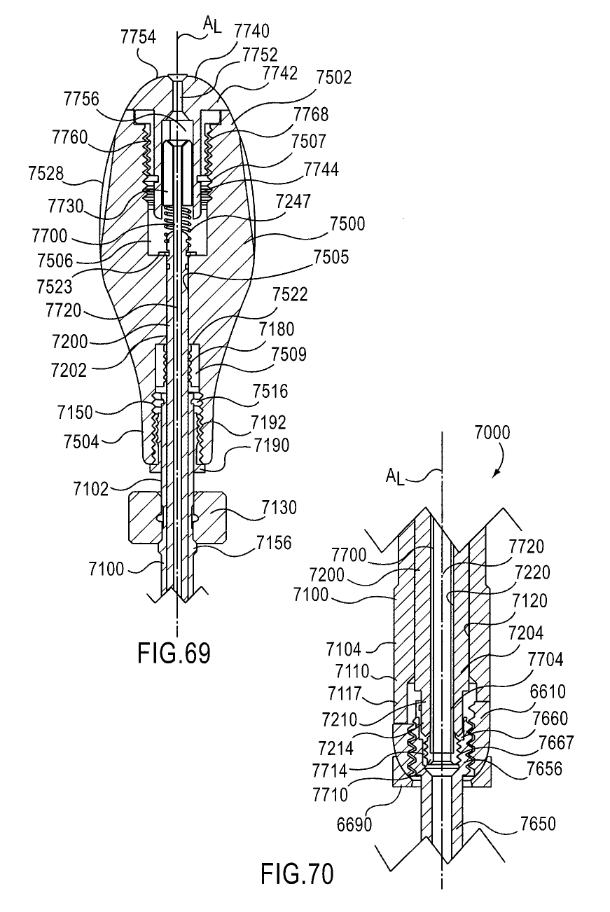

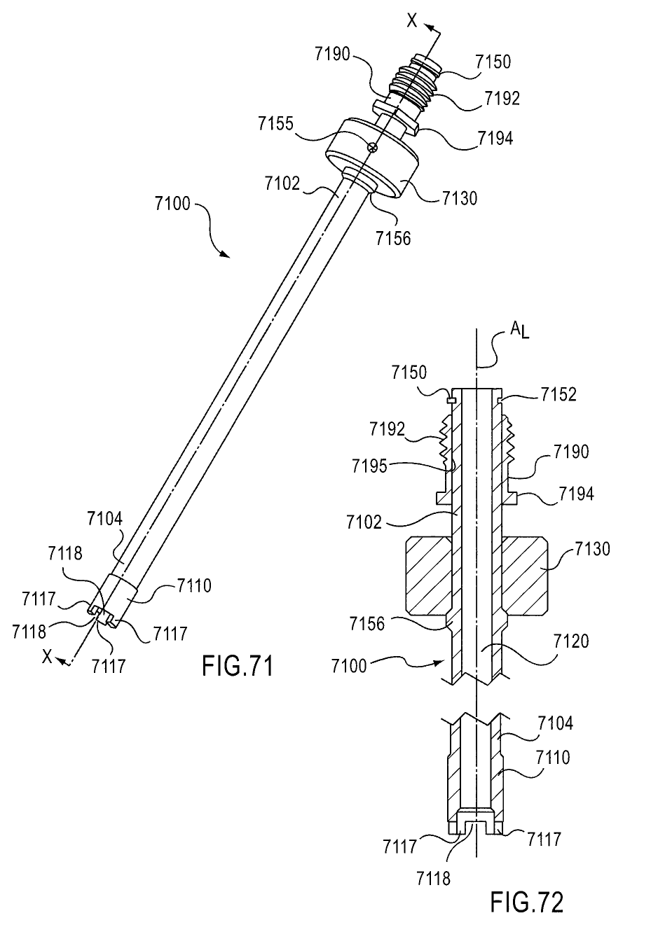

The insertion tool 7000 includes a first shaft 7100, a second shaft 7200, a third shaft 7700, and a handle 7500. The first shaft 7100 includes a proximal end portion 7102 and a distal end portion 7104, and defines a lumen 7120 therethrough. As shown in FIGS. 69, 70 and 72, the lumen 7120 defines a longitudinal axis A_{L}. As shown in FIGS. 71-72, the proximal end portion 7102 of the first shaft 7100 includes an actuator 7130, a shoulder 7156, and a coupler 7190. The actuator 7130 is disposed about the outer surface of the first shaft 7100 such that a portion of the actuator 7130 engages the shoulder 7156 defined by the first shaft 7100. The actuator 7130 is coupled to the outer surface of the first shaft 7100 using a set screw 7155. In this manner, the actuator 7130 can be used to rotate the first shaft 7100 of the insertion tool 7000 about the longitudinal axis A_{L}. As described above, the outer surface of the actuator 7130 can include any suitable topographical features to aid in grasping and rotating the actuator 7130 and therefore the first shaft 7100.

The coupler 7190 includes a threaded portion 7192 and a flange 7194, and defines a lumen 7195 therethrough. As shown in FIGS. 71 and 72, at least a portion of the proximal end portion 7102 of the first shaft 7100 is disposed within the lumen 7195 of the coupler 7190. More particularly, a diameter of the lumen 7195 of the coupler 7190 is larger than an outer diameter of the proximal end portion 7102 of the first shaft 7100 such that the first shaft 7100 can rotate within and move axially with respect to the coupler 7190. The outer surface of the proximal end portion 7102 of the first shaft 7100 defines a groove 7152 within which a retaining ring 7150 (e.g., a snap ring, an e-ring or the like) is disposed. In this manner, the retaining ring 7150 is maintained in a fixed axial position along the first shaft 7100. The outer diameter of the retaining ring 7150 is greater than the inner diameter of the lumen 7195 of the coupler 7190. Accordingly, when the first shaft 7100 is moved distally within the coupler 7190 through a predetermined distance, the retaining ring 7150 is configured to engage the proximal end of the coupler 7190. In this manner, the retaining ring 7150 can limit the axial movement of the first shaft 7100 within the coupler 7190. Moreover, as shown in FIG. 69 and described in more detail herein, the threaded portion 7192 of the coupler 7190 is threadedly engaged with the threaded portion 7516 of the handle 7500. Accordingly, when the coupler 7190 is coupled to the handle 7500, the retaining ring 7150 limits the axial movement of the first shaft 7100 in the distal direction relative to the handle 7500. Similarly stated, when the coupler 7190 is coupled to the handle 7500, the retaining ring 7150 prevents the first shaft 7100 from slipping out of the handle 7500.

As shown in FIGS. 67, 68 and 70, the distal end portion 7104 of the first shaft 7100 includes a nut engagement portion 7110. The distal end of the nut engagement portion 7110 of the first shaft 7100 includes a series of alternating protrusions 7117 and openings 7118 configured to matingly receive the nut 6610 of the bone fixation device 7600. Said another way, the alternating protrusions 7117 and openings 7118 of the first shaft 7100 substantially correspond to the alternating openings 6622 and protrusions 6621 of the nut 6610, as described above. In this manner, the nut 6610 can be engaged with the nut engagement portion 7110 of the first shaft 7100 such that rotation of the first shaft 7100 about the longitudinal axis A_{L}, results in rotation of the nut 6610.

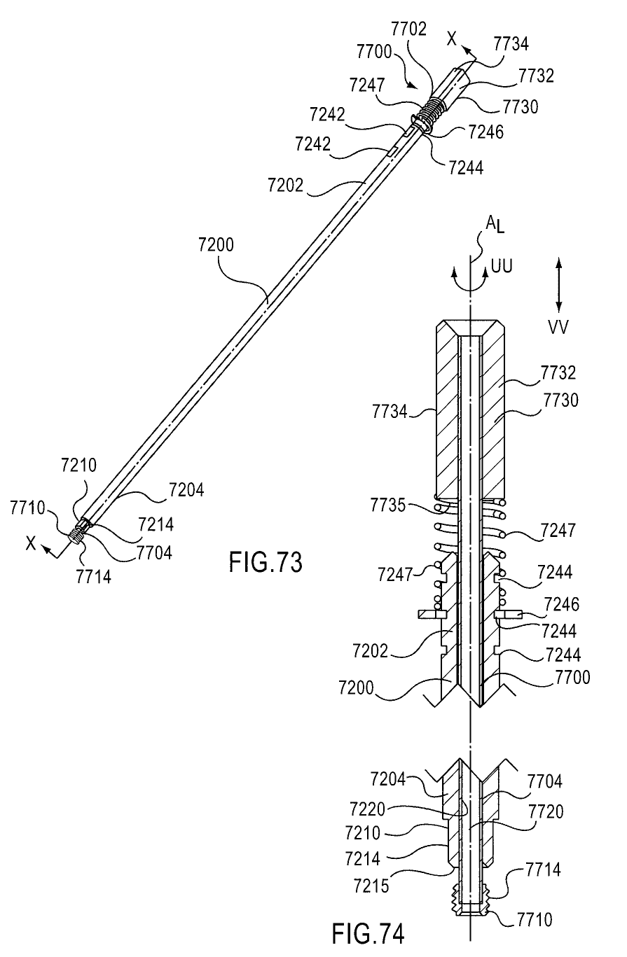

As best shown in FIGS. 73 and 74, the second shaft 7200 includes a proximal end portion 7202, a distal end portion 7204, and defines a lumen 7220 therethrough. The proximal end portion 7202 of the second shaft 7200 includes a series of flatted surfaces 7242, and defines a series of grooves 7244. Each of the grooves 7244 is configured to retain a retaining ring 7246 (e.g., a snap ring, an e-ring or the like). In this manner, the retaining ring 7246 can be maintained in one of several different fixed axial positions along the second shaft 7200. As shown in FIG. 69, the proximal end portion 7202 of the second shaft 7200 is configured to be received within a distal opening 7509 and a lumen 7505 defined by the handle 7500. When the proximal end portion 7202 of the second shaft 7200 is received within the lumen 7505, the retaining ring 7246 can contact a shoulder 7523 within the proximal opening 7506 of the handle 7500. In this manner, the retaining ring 7244 can limit the axial movement in the distal direction of the second shaft 7200 within the handle 7500. Similarly stated, the retaining ring 7244 can maintain and/or set the axial position of the first shaft 7200 within the handle 7500.

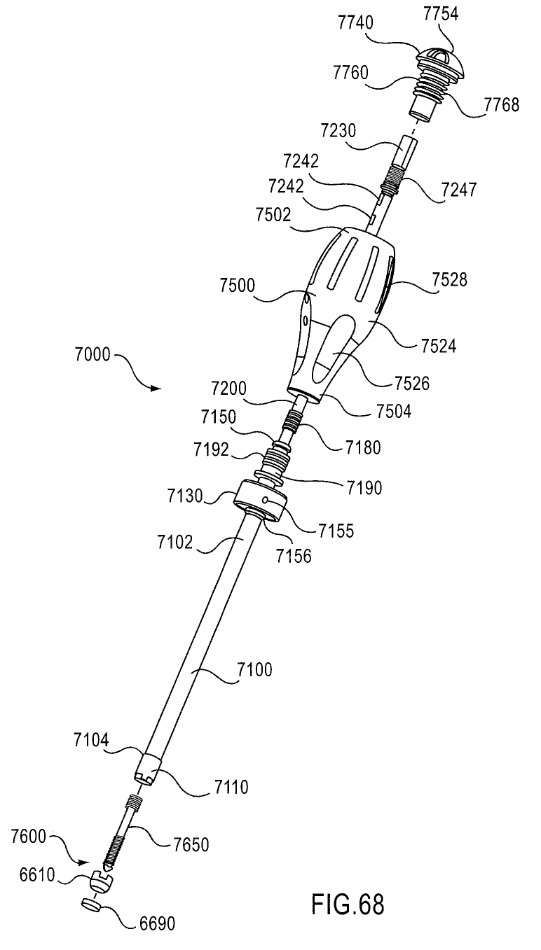

The flatted surfaces 7242 of the second shaft 7200 provide an engagement surface for a set screw 7512 (see e.g., FIG. 68) of the handle 7500. In this manner, second shaft 7200 can be coupled within the handle 7500 such that the handle 7500 can be used to rotate the second shaft 7200 of the insertion tool 7000 about the longitudinal axis A_{L}. Moreover, in this manner, the second shaft 7200 can be coupled within the handle 7500 such that axial movement of the handle 7500 results in axial movement of the second shaft 7200. Said another way, the second shaft 7200 can be coupled within the handle 7500 such that axial movement of the handle 7500 relative to the first shaft 7100 results in a substantially equivalent axial movement of the second shaft 7200 relative to the first shaft 7100.

The distal end portion 7204 of the second shaft 7200 includes an engagement portion 7210. The engagement portion 7210 includes a hexagonal shaped portion 7214 substantially corresponding to the hexagonal shaped recess 7660 defined within the engagement portion 7656 of the bone screw 7650. In this manner, the engagement portion 7210 of the second shaft 7200 can be received within the engagement portion 7656 of the bone screw 7650 such that rotation of the second shaft 7200 about the longitudinal axis A_{L} results in rotation of the bone screw 7650.

The third shaft 7700 includes a proximal end portion 7702, a distal end portion 7704, and defines a lumen 7770 therethrough. The proximal end portion 7702 of the third shaft 7700 includes an actuation portion 7730 configured to be matingly received within the dial actuator 7740. The actuation portion 7730 includes a side wall 7732 and having an outer surface 7734 and a distal end surface 7735. The outer surface 7734 has a flatted shape (e.g., a substantially square shape) corresponding to the shape of the opening 7756 defined by the dial actuator 7740. In this manner, the actuation portion 7730 of the third shaft 7700 can be received within the dial actuator 7740 such that rotation of the dial actuator 7740 about the longitudinal axis A_{L} results in rotation of the third shaft 7700.

The distal end portion 7704 of the third shaft 7700 includes an engagement portion 7710. The engagement portion 7710 includes a threaded portion 7714 that can be matingly engaged with the female threads 7667 within the engagement portion 7656 of the bone screw 7650. Said another way, the thread pitch of the threaded portion 7714 of the third shaft 7700 is substantially the same as the thread pitch of the female threads 7667 of the bone screw 7650. In this manner, the third shaft 7700, and therefore the insertion tool 7000, can be removably coupled to the bone screw 7650. Moreover, as described in more detail below, this arrangement allows the third shaft 7700 to remain coupled to the bone screw 7650 throughout the entire insertion process.

As shown in FIGS. 69, 70 and 73, at least a portion of the third shaft 7700 is disposed within the lumen 7220 of the second shaft 7200 such that the third shaft 7700 and the second shaft 7200 are coaxial about the longitudinal axis A_{L}. The portion of the third shaft 7700 is disposed within the lumen 7220 of the second shaft 7200 such that the third shaft 7700 can rotate about the longitudinal axis A_{L} relative to the second shaft 7200, as shown by the arrow UU in FIG. 74. Moreover, the portion of the third shaft 7700 is disposed within the lumen 7220 of the second shaft 7200 such that the third shaft 7700 can move axially (i.e., along the longitudinal axis A_{L}, as shown by the arrow VV in FIG. 74) relative to the second shaft 7200.

The portion of the third shaft 7700 is disposed within the lumen 7220 of the second shaft 7200 such that the actuation portion 7730 of the third shaft 7700 is spaced proximally apart from the proximal end portion 7702 of the second shaft 7200 and the engagement portion 7710 of the third shaft 7700 is spaced distally from the distal end surface 7215 of the second shaft 7200. The outer diameter of the engagement portion 7710 of the third shaft 7700 is greater than the inner diameter of the lumen 7220 of the second shaft 7200. Accordingly, when the third shaft 7700 is moved within the second shaft 7200 axially in the proximal direction, the engagement portion 7710 is configured to contact the distal end surface 7215 of the second shaft 7200 to limit further proximal movement of the third shaft 7700 within the second shaft 7200 (see e.g., FIG. 70). Similarly, the size of the actuation portion 7730 of the third shaft 7700 is greater than the inner diameter of the lumen 7220 of the second shaft 7200. Accordingly, when the third shaft 7700 is moved within the second shaft 7200 axially in the distal direction, the actuation portion 7730 is configured to contact the proximal end portion 7202 of the second shaft 7200 to limit further distal movement of the third shaft 7700 within the second shaft 7200 (see e.g., FIG. 70).

A spring 7247 is disposed between the distal end surface 7735 of the actuation portion 7730 and the retaining ring 7246 of the second shaft 7200. In this manner, the third shaft 7700 is biased within the second shaft 7200 axially in the proximal direction. This arrangement allows the third shaft 7700 to be freely rotated within the second shaft 7200 while the engagement portion 7710 of the third shaft 7700 is maintained in contact with the distal end surface 7215 of the second shaft 7200. In this manner, when the bone fixation device 7600 is coupled to the third shaft 7700, the engagement portion 7656 of the bone screw will be biased against the engagement portion 7210 of the second shaft 7200. The ease with which the third shaft 7700 can be rotated within the second shaft 7200 is a function of, among other things, the amount of force applied by the spring 7247 to the third shaft 7700 and the second shaft 7200 (i.e., the biasing force). As the biasing force increases, the frictional force between the engagement portion 7710 of the third shaft 7700 and the distal end surface 7215 of the second shaft 7200 increases, which resists the rotation of the third shaft 7700 within the second shaft 7200. Although the biasing force cannot be adjusted during use in the embodiment shown in FIGS. 67-76, the biasing force can be changed by changing the spring constant of the spring 7247 and/or by changing the compression of the spring 7247 (i.e., the difference between the free length of the spring 7247 and the compressed length of the spring 7247). The compression of the spring 7247 can be changed by moving the position of the retaining ring 7246 on the outer surface of the second shaft 7200 (e.g., by changing the groove 7244 within which the retaining ring 7246 is disposed). Although third shaft 7700 is shown as being is biased within the second shaft 7200, in other embodiments, the third shaft 7700 can be movably disposed within the second shaft 7200 without a spring.

As shown in FIGS. 68-71, at least a portion of the second shaft 7200 is disposed within the lumen 7120 of the first shaft 7100 such that the first shaft 7100 and the second shaft 7200 are coaxial about the longitudinal axis A_{L}. The portion of the second shaft 7200 is disposed within the lumen 7120 of the first shaft 7100 such that the second shaft 7200 can rotate about the longitudinal axis A_{L} relative to the first shaft 7100. Moreover, the portion of the second shaft 7200 is disposed within the lumen 7120 of the first shaft 7100 such that the second shaft 7200 can move axially (i.e., along the longitudinal axis A_{L}) relative to the first shaft 7100. As described above, the retaining ring 7150 of the first shaft 7100 is configured to limit the axial movement of the first shaft 7100 within the coupler 7190 and/or relative to the handle 7500. Because the second shaft 7200 is fixedly coupled to the handle 7500, the retaining ring 7150 and the coupler 7190 therefore limit the axial movement of the first shaft 7100 about the second shaft 7200.

As shown in FIGS. 68-70, the handle 7500 includes a proximal portion 7502 and a distal portion 7504 and defines a lumen 7505 therethrough configured to receive a guide member (not shown) of the types shown and described herein. The proximal portion 7502 of the handle 7500 defines a proximal opening 7506 that is coaxial with the longitudinal axis A_{L} and in fluid communication with the lumen 7505. The proximal opening 7506 is configured to receive the dial actuator 7740. More particularly, the inner surface that defines the proximal opening 7506 includes a threaded portion 7507 configured to engage a corresponding threaded portion 7768 of the coupler 7760 that retains the dial actuator 7740 within the proximal opening 7506 of the handle 7500.

The inner surface that defines the proximal opening 7506 includes a shoulder 7523 configured to contact the retaining ring 7246 of the second shaft 7200 when the proximal end portion 7202 of the second shaft is assembled within the handle 7500. In this manner, as described above, the retaining ring 7246 and the shoulder 7523 can cooperatively limit the axial movement of the second shaft 7200 within the handle 7500. Similarly stated, the retaining ring 7246 and the shoulder 7523 can cooperatively maintain and/or set the axial position of the first shaft 7200 within the handle 7500.

The distal portion 7504 of the handle 7500 defines a distal opening 7509 that is coaxial with the longitudinal axis A_{L} and in fluid communication with the lumen 7505. As shown in FIG. 69, the distal opening 7509 is configured to receive a portion of the first shaft 7100 and a portion of the second shaft 7200. Moreover, as described above, the surface defining the distal opening 7509 includes a threaded portion 7516 configured to engage the threaded portion 7192 of the coupler 7190 that is used to retain the first shaft 7100 within the distal opening 7509. Said another way, the surface defining the distal opening 7509 defines a female threaded portion 7516 configured to engage the corresponding male threaded portion 7192 of the coupler 7190. Because the first shaft 7100 can rotate within and/or move axially relative to the coupler 7190, this arrangement permits the first shaft 7100 to be retained within the handle 7500, while allowing the first shaft 7100 to rotate about the longitudinal axis A_{L} relative to the handle 7500 and/or the second shaft 7200 and move axially relative to the handle 7500 and/or the second shaft 7200.

The inner surface that defines the distal opening 7509 includes a shoulder 7522. As shown in FIG. 69, when the proximal end portion 7102 of the first shaft 7100 is disposed within the distal opening 7509, a spring 7180 is disposed between the shoulder 7522 and the proximal end portion 7102 of the first shaft. In this manner, the first shaft 7100 is biased within the handle 7500 in the distal direction. Moreover, this arrangement biases the first shaft 7100 distally relative to the second shaft 7200. Accordingly, as shown in FIG. 70, when the engagement portion 7210 of the second shaft 7200 is disposed within the hexagonal shaped recess 7660 of the bone screw 7650, the nut engagement portion 7110 of the first shaft 7100 is biased distally relative to the second shaft 7200 such that the nut engagement portion 7110 maintains engagement with the nut 6610.

The handle 7500 includes a transverse lumen (not shown) that is substantially normal to the longitudinal axis A_{L}. The transverse lumen intersects the lumen 7505 adjacent one of the flatted surfaces 7242 of the second shaft 7200, and is configured to threadedly receive a set screw 7512. In this manner, the second shaft 7200 can be coupled within the handle 7500 by the set screw 7512 such that the handle 7500 can be used to rotate the second shaft 7200 about the longitudinal axis A_{L}. The handle 7500 includes an outer surface 7524, which includes a bulb-shaped portion 7528 and multiple flats 7526. Accordingly, the outer surface 7524 of the handle 7500 is configured to be grasped and/or manipulated by the user, for example, to rotate the first shaft 7100 and/or the second shaft 7200 about the longitudinal axis A_{L}.

The dial actuator 7740 includes a proximal portion 7742 and a distal portion 7744 and defines a lumen 7752 therethrough configured to receive a guide member (not shown). As shown in FIG. 69, the dial actuator is configured to be disposed within the proximal opening 7506 of the handle 7500 such that the lumen 7752 is substantially coaxial with the longitudinal axis A_{L} and the lumen 7720 of the third shaft 7200. In this manner, a guide member (not shown) can be disposed within and/or through the lumen 7752 and into the lumen 7720 of the third shaft 7200.

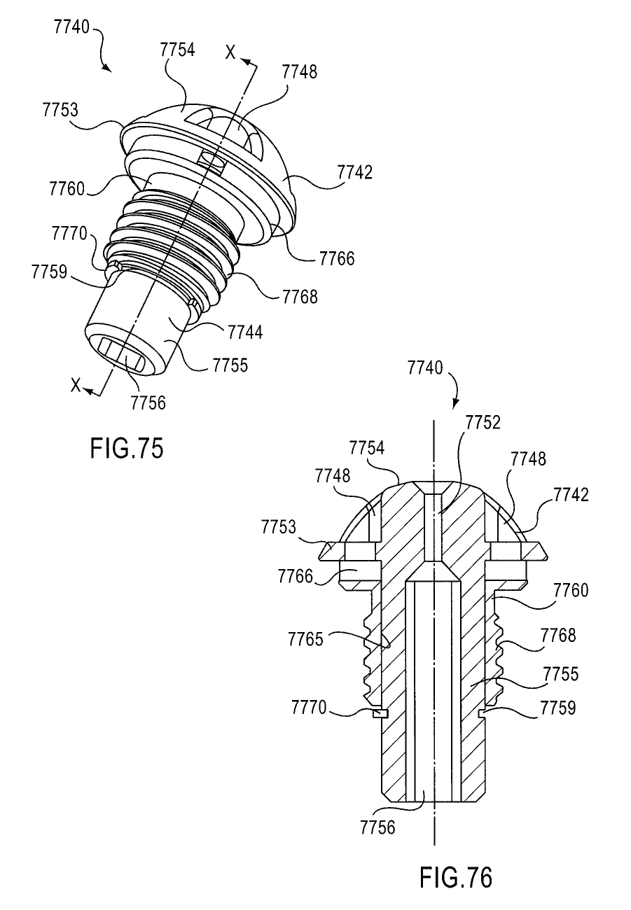

As shown in FIGS. 75 and 76, the proximal portion 7742 of the dial actuator 7740 includes an outer surface 7754 and a flange 7753. The outer surface 7754 defines a series of recesses 7748 to aid in grasping and rotating the dial actuator 7740 (and therefore the third shaft 7700) within the handle 7500. Although shown as including recesses 7748, in other embodiments, the outer surface 7754 of the dial actuator 7740 can include any suitable topographical features to aid in grasping and rotating the dial actuator 7740.

The distal portion 7744 of the dial actuator 7740 includes a side wall 7755 that defines an opening 7756. The portion of the side wall 7755 defining the opening 7756 has a flatted shape (e.g., a substantially square shape) corresponding to the shape of the actuation portion 7730 of the third shaft 7700. In this manner, as described above, the actuation portion 7730 of the third shaft 7700 can be received within the dial actuator 7740 such that rotation of the dial actuator 7740 about the longitudinal axis A_{L} results in a corresponding rotation of the third shaft 7700. Said another way, the actuation portion 7730 of the third shaft 7700 can be received within the dial actuator 7740 such that the rotational motion of the third shaft 7700 relative to the dial actuator 7740 is limited. Moreover, the actuation portion 7730 of the third shaft 7700 can be received within the dial actuator 7740 such that the actuation portion 7730 can move axially within the opening 7756. As described above, the axial motion of the third shaft 7700 relative to the dial actuator 7740 and/or the second shaft 7200 is limited by the interference relationship of the engagement portion 7710 of the third shaft 7700 and the distal end surface 7215 of the second shaft 7200, and/or the actuation portion 7730 of the third shaft 7700 and the proximal end portion 7202 of the second shaft 7200. In this manner, although the second shaft 7200 is fixedly coupled to the handle 7500, the third shaft 7700 can move, both axially and rotationally, relative to the handle 7500.

The dial actuator 7740 is coupled to and/or retained within the handle 7500 by the coupler 7760. As shown in FIGS. 75 and 76, the coupler 7760 includes a threaded portion 7768 and a flange 7766, and defines a lumen 7765 therethrough. At least a portion of the distal portion 7744 of the dial actuator 7740 is disposed within the lumen 7765 of the coupler 7760. More particularly, a size (e.g. an inner diameter) of the lumen 7765 of the coupler 7760 is larger than an outer diameter of the distal portion 7744 of the dial actuator 7740 such that the dial actuator 7740 can rotate within and move axially with respect to the coupler 7760. The outer surface of the side wall 7755 of the dial actuator 7740 defines a groove 7759 within which a retaining ring 7770 (e.g., a snap ring, an e-ring or the like) is disposed. In this manner, the retaining ring 7770 is maintained in a fixed axial position along the dial actuator 7740. The outer diameter of the retaining ring 7770 is greater than the inner diameter of the lumen 7765 of the coupler 7760. Accordingly, when the dial actuator 7740 is moved proximally within the coupler 7760 through a predetermined distance, the retaining ring 7770 is configured to engage the distal end of the coupler 7760. In this manner, the retaining ring 7770 can limit the axial movement of the dial actuator 7740 within the coupler 7760. Moreover, as shown in FIG. 69, the threaded portion 7768 of the coupler 7760 is threadedly engaged with the threaded portion 7507 of the handle 7500. Accordingly, when the coupler 7760 is coupled to the handle 7500, the retaining ring 7770 limits the axial movement of the dial actuator 7740 in the proximal direction relative to the handle 7500. Similarly stated, when the coupler 7760 is coupled to the handle 7500, the retaining ring 7770 prevents the dial actuator 7740 from slipping out of the handle 7500.

As described above, the third shaft 7700 can be rotated within the second shaft 7200 by rotating the dial actuator 7740 relative to the handle 7500 about the longitudinal axis A_{L}. In this manner, the bone fixation device 7600 can be threadedly coupled to the insertion tool 7000 prior to inserting of the bone fixation device 7600 into the body. More particularly, the bone fixation device 7600 can be coupled to the insertion tool 7000 by first inserting the engagement portion 7210 of the second shaft 7200 into hexagonal shaped recess 7660 defined within the engagement portion 7656 of the bone screw 7650. The nut engagement portion 7110 of the first shaft 7100 can then be aligned rotationally with nut 6610, such that the alternating protrusions 7117 and openings 7118 of the nut engagement portion 7110 matingly engage the alternating protrusions 6621 and openings 6622 of the nut 6610. As described above, the first shaft 7100 is biased in the distal direction relative to the second shaft 7200. Accordingly, the biasing force from the spring 7180 helps to maintain the engagement between the nut engagement portion 7110 of the first shaft 7100 and the nut 6610. The third shaft 7700 can then be rotated within the second shaft 7200 and the first shaft 7100 such that the threaded portion 7714 of the third shaft 7700 is threaded into the corresponding threaded portion 7667 of the bone screw 7650. In this manner, the insertion tool 7000 and the bone fixation device 7600 can be placed in a first configuration (see e.g., FIG. 71). Said another way, after the bone fixation device 7600 is coupled to the insertion tool 7000, and the nut 6610 is not tightened on the bone screw 7650, the insertion tool 7000 and the bone fixation device 7600 are in a first configuration.

When the insertion tool 7000 and the bone fixation device 7600 are in the first configuration, the distal end portion of the insertion tool 7000 and the bone fixation device 7600 can be inserted into the body and positioned adjacent a target bone tissue, as described above. Although the insertion tool 7000 is not shown and described as including a guide member, in other embodiments, the insertion tool can include a guide member, similar to the guide wire 5550, to locate the target bone tissue and/or define a passageway within the bone tissue as described above. When the distal end portion 7654 of the bone screw 7650 is disposed against the bone tissue and with the insertion tool 7000 and the bone fixation device 7600 in the first configuration, the bone screw 7650 can be threaded into the bone tissue by rotating the second shaft 7200 about the longitudinal axis A_{L}.

In some embodiments, the bone screw 7650 can be threaded into the bone tissue by rotating the first shaft 7100, the second shaft 7200, and the third shaft 7700 substantially simultaneously. In this manner, when the bone screw 7650 is advanced into the bone tissue, the nut 6610 remains in a substantially constant axial position relative to the bone screw 7650 (e.g., the nut 6610 is not tightened onto the bone screw 7650). Similarly, when the bone screw 7650 is advanced into the bone tissue, the third shaft 7700 remains threadedly engaged with the bone screw 7650. In some embodiments, the first shaft 7100 and the second shaft 7200 can be rotated simultaneously by rotating both the handle 7500 and the actuator 7130. In other embodiments, the insertion tool 7000 can include a locking mechanism, such as the locking mechanism 5300 shown and described above, to allow the user to simultaneously rotate the first shaft 7100 and the second shaft 7200 by rotating only the handle 7500. In yet other embodiments, the insertion tool 7000 can include a frictional coupling between the first shaft 7100 and the second shaft 7200 that causes the first shaft 7100 to rotate with the second shaft 7200 until an external force opposing the rotation of the first shaft 7100 exceeds the frictional force caused by such a frictional coupling.

Similarly, the third shaft 7700 and the second shaft 7200 can be rotated simultaneously by rotating the handle 7500. The force imparted by the engagement between the threaded portion 7714 of the third shaft and the threaded portion 7767 of the bone screw 7650 can selectively limit the rotational motion of the bone screw 7650 relative to the third shaft 7700. Similarly stated, the frictional force caused by the threaded engagement of the third shaft 7700 and the bone screw 7650 opposes the rotational motion of the bone screw 7650 relative to the third shaft 7700. Accordingly, when the bone screw 7650 is rotated about the longitudinal axis A_{L}, the third shaft 7700 is simultaneously rotated.

After the bone screw 7650 is inserted within the targeted bone tissue, the user can pull the handle 7500 proximally to assess the quality of the of the engagement between the bone screw 7650 and the bone tissue. Said another way, because the bone screw 7650 is threadedly coupled to the insertion tool 7000, after the bone screw 7650 is inserted within the targeted bone tissue, the user can pull the handle 7500 proximally to get tactile feedback associated with the quality of the bone purchase.

Moreover, after the bone screw 7650 is threaded into the bone tissue, the insertion tool 7000 and the bone fixation device 7600 can be moved from the first configuration to a second configuration (not shown in FIGS. 67-76) by rotating the first shaft 7100 about the second shaft 7200. Said another way, the insertion tool 7000 and the bone fixation device 7600 can be moved from the first configuration to the second configuration by rotating the nut 6610 relative to the bone screw 7650 (e.g., by “tightening” the nut 6610). The first shaft 7100 can be rotated about the second shaft 7200 by maintaining the rotational position of the handle 7500 and applying a rotational force to the first shaft 7100 via the actuator 7130.

When the first shaft 7100 is rotated about the second shaft 7200, the nut 6610 moves axially relative to the bone screw 7650. The biasing force from the spring 7180 also moves the first shaft 7100 distally along the longitudinal axis A_{L} relative to the second shaft 7200. In this manner, the first shaft 7100 remains engaged with the nut 6610 when the nut 6610 is tightened and/or loosened about the bone screw 7650. Moreover, unlike the operation of the insertion tool 6000 described above, the bone fixation device 7600 remains coupled to the insertion tool 7000 as the insertion tool 7000 and the bone fixation device 7600 are moved from the first configuration to a second configuration. Said another way, the threaded portion 7714 of the third shaft remains engaged with the threaded portion 7767 of the bone screw 7650 as the insertion tool 7000 and the bone fixation device 7600 are moved from the first configuration to a second configuration.

After the bone fixation device 7600 is inserted within the targeted bone tissue and moved from the first configuration to the second configuration, the third shaft 7700 can be decoupled from the bone screw 7650 by rotating the dial actuator 7760 about the longitudinal axis A_{L} in a direction opposite that used to couple the third shaft 7700 to the bone screw 7650. The insertion tool 7000 can then be removed from the body.

In some embodiments, an insertion tool can include a sheath having a portion configured to be disposed about at least a portion of the bone fixation device during at least a portion the insertion operation. In this manner the sheath can prevent the bodily tissue adjacent the insertion path from being damaged by the bone fixation device (e.g., by the threads of the bone screw) during insertion. Moreover, in some embodiments, the sheath can also be used to removably couple the bone fixation device to the insertion tool.

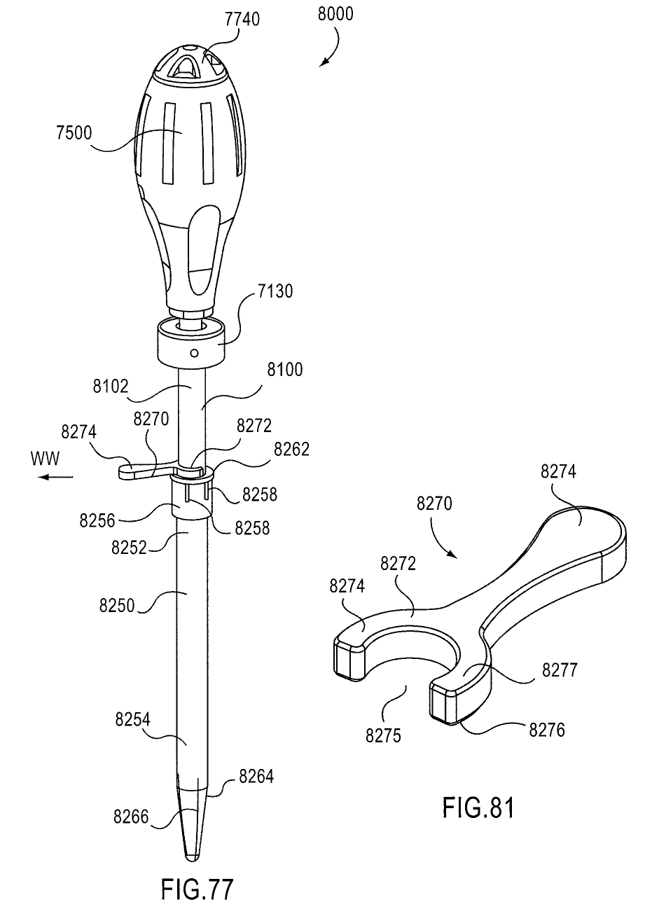

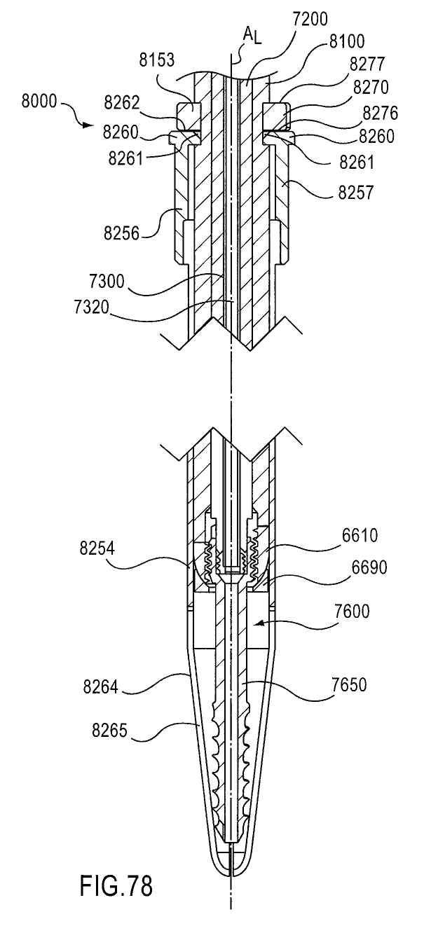

One such embodiment is shown in FIGS. 77-81, which show an insertion tool 8000 according to an embodiment of the invention as used with the bone fixation device 7600 shown and described above. The insertion tool 8000 includes a first shaft 8100, a second shaft 7200, a third shaft 7700, and a handle 7500, as shown and described above with reference to FIGS. 67-76. The first shaft 8100 is similar to the first shaft 7100 shown and described above, except, as shown in FIG. 78, the outer surface of the first shaft 8100 defines a groove 8153 within which a portion of a retention member 8270 is disposed. Accordingly, the remaining details of the insertion tool 8000 and the first shaft 8100 of the insertion tool 8000 are not described in detail below.

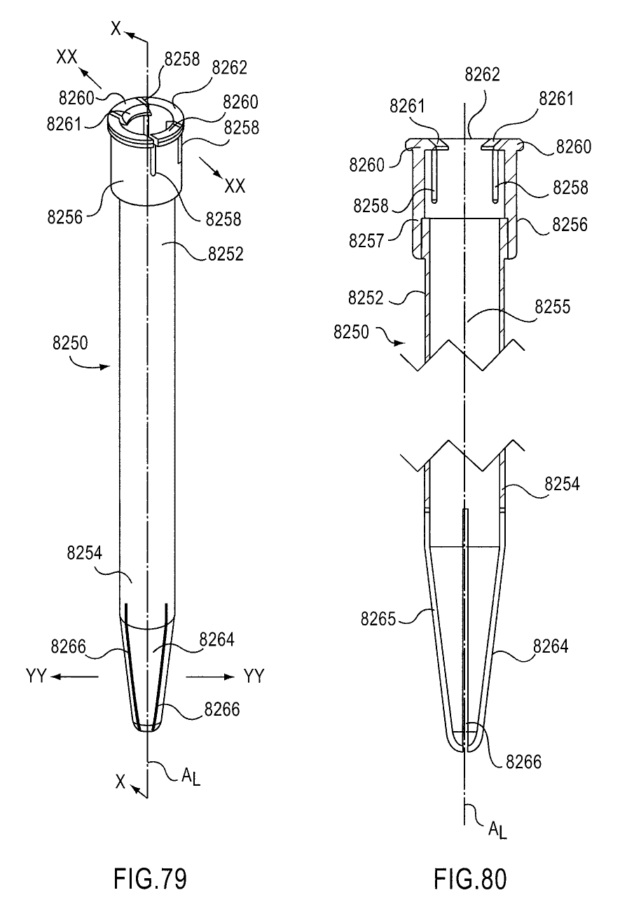

Additionally, the insertion tool 8000 includes a sheath 8250. The sheath 8250 includes a proximal end portion 8252 and a distal end portion 8254, and defines a lumen 8255 (see FIG. 80) therethrough. The proximal end portion 8252 includes an engagement portion 8256 configured to removably couple the sheath 8250 to the first shaft 8100. The engagement portion 8256 of the sheath 8250 includes a pair of opposing protrusions 8260 and a proximal end surface 8262. Each of the protrusions 8260 includes a tapered surface 8261. The engagement portion 8256 of the sheath 8250 defines a four elongate openings 8258. The elongate openings 8258 are substantially parallel to the longitudinal axis A_{L}, and are defined adjacent each of the opposing protrusions 8260. Accordingly, as shown by the arrows XX in FIG. 79, the opposing protrusions 8260 can move relative to each other in a direction substantially normal to the longitudinal axis A_{L}. Similarly stated, this arrangement allows the opposing protrusions 8260 to be spread apart when subjected to an outward force. In this manner, as described in more detail herein, the engagement portion 8256 of the sheath 8250 can be decoupled from the first shaft 8100.

The distal end portion 8254 of the sheath 8250 includes a tapered portion 8264 having a side wall 8265. The side wall 8265 defines four elongate openings 8266 that extend substantially longitudinally along the tapered portion 8264. The elongate openings 8266 are substantially equally spaced apart radially (i.e., the elongate openings 8266 are defined with approximately ninety degrees of spacing between each of the elongate openings 8266). Accordingly, as shown by the arrows YY in FIG. 79, portions of the tapered portion 8264 can move relative to each other in a direction substantially normal to the longitudinal axis A_{L}. In this manner, as described in more detail herein, the tapered portion 8264 of the sheath 8250 can be expanded to be moved in the proximal direction over the bone fixation device 7600. Said another way, as described in more detail herein, the size of the portion of the lumen 8255 within the tapered portion 8264 can be increased such that the tapered portion 8264 can be moved about the bone fixation device 7600 when the bone fixation device 7600 is inserted from the proximal opening of the sheath 8250 and moved in the distal direction.

As shown in FIGS. 77 and 78, the sheath 8250 can be coupled to the outer surface of the first shaft 8100 of the insertion device 8000 when the bone fixation device 7600 is coupled to the insertion tool 8100. Moreover, the sheath 8250 can be selectively maintained in position about the first shaft 8100 by a retention member 8270. As shown in FIG. 81, the retention member 8270 includes a first end 8272 and a second end 8274. The first end 8272 includes a side wall 8274 that includes a proximal surface 8277 and a distal surface 8276, and defines an opening 8275.

When the sheath 8250 is coupled to the first shaft 8100, at least a portion of the first shaft 8100 is disposed within the lumen 8255 of the sheath 8250 and the protrusions 8260 of the engagement portion 8256 are disposed within the groove 8153 defined by the first shaft 8100. Additionally, at least a portion of the side wall 8274 of the retention member 8270 is disposed within the groove 8153. More particularly, the protrusions 8260 of the engagement portion 8256 are disposed distally from the side wall 8274 of the retention member 8270 such that the distal surface 8276 of retention member 8270 is in contact with the proximal surface 8262 and/or the protrusions 8260, and the proximal surface 8277 of the retention member 8270 is in contact with a portion of the side wall of the first shaft 8100 that defines the groove 8153. In this manner, the retention member 8270 and the groove 8153 cooperatively limit the axial motion of the sheath 8250 relative to the first shaft 8100.

Moreover, when the sheath 8250 is coupled to the first shaft 8100, the bone fixation device 7600 is disposed within the tapered portion 8264 of the sheath 8250 such that at least a portion of the side wall 8265 of the tapered portion 8264 is in contact with at least a portion of the bone fixation device 7600. In this manner, the sheath 8250 can prevent the bodily tissue adjacent the insertion path from being damaged by the bone fixation device (e.g., by the threads of the bone screw) during insertion. Moreover, although the insertion tool 8000 is removably coupled to the bone fixation device 7600 by a threaded coupling, as described above, in some embodiments, the sheath 8250 can also be used to removably couple the bone fixation device 7600 to the insertion tool 8100.

In use, the bone fixation device 7600 and the distal portion of the insertion tool 8000 can be inserted into the body and positioned adjacent target bone tissue, when the sheath 8250 is disposed about at least a portion of the first shaft 8100 and the bone fixation device 7600. During the insertion operation, the sheath 8250 is prevented from moving proximally by the retention member 8270. Similarly stated, the engagement between the retention member 8270 and the engagement portion 8256 of the sheath 8250 within the groove 8153 of the first shaft 8100 are sufficient to resist proximal motion of the sheath 8250 during the insertion operation. Additionally, the tapered portion 8264 of the sheath 8250 can assist in the insertion process by reducing the reaction force of the tissue on the sheath 8250 in the proximal direction during the insertion operation. In some embodiments, for example, the tapered portion 8264 can include a lubricant to reduce the friction during insertion.

When the distal end portion the bone screw 7650 is disposed against the bone tissue as desired, the sheath 8250 can then be moved proximally relative to the first shaft 8100 to expose at least a portion of the bone screw 7650 and/or to allow the bone screw 7650 to be threaded into the bone tissue, as described above. The sheath 8250 can be moved by first removing the retention member 8270 from the first shaft 8100. The retention member 8270 can be removed by grasping the second end 8274 and pulling in the direction as shown by the arrow WW in FIG. 77.

The user can then move the sheath 8250 proximally about the first shaft 8100. The proximal motion of the sheath 8250 causes the tapered surface 8261 of each protrusion 8260 to contact the portion of the side wall of the first shaft 8100 that defines the groove 8153. Accordingly, the force moving the sheath 8250 proximally is applied to the protrusions 8260 via the tapered surfaces 8261. Because the tapered surfaces 8261 are angled with respect to the longitudinal axis A_{L}, a component of the force transmitted via the tapered surfaces 8261 to the protrusions 8260 has an outward direction, as shown by the arrow XX in FIG. 79. Said another way, because the tapered surfaces 8261 are at an acute angle (e.g., an angle between zero and ninety degrees) with respect to the longitudinal axis A_{L}, a component of the force transmitted via the tapered surfaces 8261 to the protrusions 8260 has an outward direction, as shown by the arrow XX in FIG. 79. Accordingly, the protrusions 8260 can be moved such that they are no longer within the groove 8153, and the sheath 8250 can be moved proximally relative to the outer shaft 8100. When the sheath 8250 moves proximally, portions of the tapered portion 8264 of the distal end portion 8254 of the sheath 8250 can move relative to each other as shown by the arrows YY in FIG. 79. In some embodiments, the sheath 8250 can move proximally a distance equal to or greater than the length of the bone fixation device 7600. In this manner, the proximal motion of the sheath 8250 can expose the bone fixation device 7600.

The sheath 8250 can be constructed from any suitable biocompatible material. For example, in some embodiments, the sheath 8250 can be constructed from a flexible polymer. Such construction can allow the opposing protrusions 8260 and/or the tapered portion 8264 to flexibly move as described above, and return to their original shape. Similarly stated, the sheath 8250 can be constructed from a polymer such that the opposing protrusions 8260 and/or the tapered portion 8264 can move elastically when the sheath is disposed about and/or removed from the first shaft 8100.

Although various embodiments of the invention have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above. Thus, the breadth and scope of the invention should not be limited by any of the above-described embodiments. While the invention has been particularly shown and described with reference to specific embodiments thereof, it will be understood that various changes in form and details may be made.

For example, although the first shaft 5100 is shown and described above as being threadedly coupled to the second shaft 5200, in other embodiments, an insertion tool can include a first shaft that is coupled to a second shaft via a ratchet mechanism. In this manner, the first shaft can be rotated about the second shaft in an incremental and/or controlled fashion. Accordingly, an insertion tool having such an arrangement can be used to tighten a nut about a bone screw in an incremental and/or controlled fashion. For example in some embodiments, an insertion tool can include a first shaft that is coupled to a second shaft via a ratchet mechanism that can selectively allow unidirectional rotation of the first shaft about the second shaft.

Similarly, although the guide wire 5550 is shown as being threadedly coupled to the handle, in other embodiments, an insertion tool can include a guide wire that is coupled to a handle via a ratchet mechanism. In this manner, the guide wire can moved relative to the handle in an incremental and/or controlled fashion. Accordingly, an insertion tool having such an arrangement can be used to advance the guide wire into a target bone tissue in an incremental and/or controlled fashion.

In some embodiments, an insertion tool can include a rotation-limiting mechanism configured to limit the rotation of the first shaft about the second shaft. In this manner, the rotation-limiting mechanism can reduce the likelihood that a nut will be overtightened about a bone screw. For example, in some embodiments, an insertion tool can include a mechanism (e.g., a shoulder, protrusion, or any other suitable hard stop) configured to limit the number of rotations about which the first shaft can rotate relative to the second shaft. In other embodiments, an insertion tool can include a mechanism configured to limit the torque with which the first shaft is rotated about the second shaft. In this manner, when the first shaft is used to tighten a nut about a bone screw, as described above, the torque-limiting mechanism can prevent further tightening (i.e., further rotation of the first shaft about the second shaft) when the nut is tightened onto the bone screw at a predetermined torque. In some such embodiments, the torque-limiting mechanism can be adjustable. In this manner, the user can set the predetermined torque threshold as desired for the operation (e.g., based on the type of bone tissue, the condition of the bone tissue, the type of bone screw, or the like).

Similarly, although the second shaft 5200 is shown and described above as being fixedly coupled to the handle 5500, in other embodiments, an insertion tool can include a second shaft (e.g., a screw driver shaft) coupled to a handle via a torque-limiting mechanism. In this manner, the torque-limiting mechanism can reduce the likelihood that a bone screw will be overtightened within a target bone tissue.

Although various embodiments have been described as having particular features and/or combinations of components, other embodiments are possible having a combination of any features and/or components from any of embodiments as discussed above. For example, one such embodiment includes an insertion tool similar to the insertion tool 5000 and a sheath similar to the sheath 8250.

What is claimed is:

1. A method, comprising:
inserting a bone fixation device into a patient's body, the bone fixation device including a first member and a second member movably coupled to the first member;
defining a passageway within a bone tissue, after the inserting and while the bone fixation device is disposed within the patient's body;
disposing at least a portion of the first member of the bone fixation device within the bone tissue along the passageway; and
moving the second member of the bone fixation device relative to the first member of the bone fixation device.

2. The method of claim 1, wherein:
the inserting is performed using an insertion tool; and
at least one of the defining, the disposing or the moving is performed using the insertion tool.

3. The method of claim 1, further comprising:
coupling the bone fixation device to a distal end portion of an insertion tool, before the inserting and while the distal end portion of the insertion tool is outside of the patient's body, such that distal movement of the bone fixation device along its longitudinal axis relative to the distal end portion of the insertion tool is limited.

4. The method of claim 1, wherein the defining includes advancing a guide wire into the bone tissue, the method further comprising:
removing the guide wire from the passageway before the disposing.

5. The method of claim 1, wherein:
the inserting is performed percutaneously through an incision; and
at least one of the defining, the disposing or the moving is performed percutaneously through the incision.

6. The method of claim 1, wherein:
the inserting includes inserting a distal end portion of an insertion tool into the patient's body, the bone fixation device being coupled to the distal end portion of the insertion tool during the inserting;
the defining includes advancing a first shaft of the insertion tool into the bone tissue;
the disposing includes rotating a second shaft of the insertion tool such that at least the first member of the bone fixation device is threadedly disposed within the passageway, the second shaft different from the first shaft; and
the moving includes rotating a third shaft of the insertion tool relative to the second shaft of the insertion tool such that at least the second member of the bone fixation device rotates relative to the first member of the bone fixation device, the third shaft being different from the first shaft and the second shaft.

7. The method of claim 1, wherein:
the defining includes advancing a first shaft of an insertion tool into the bone tissue; and
the disposing includes rotating a second shaft of the insertion tool such that at least the first member of the bone fixation device rotatably advances into the passageway, the second shaft different from the first shaft, the insertion tool being disposed within the patient's body between the defining and the disposing.

8. The method of claim 1, wherein the moving includes rotating the second member of the bone fixation device relative to the first member of the bone fixation device.

9. The method of claim 1, wherein:
the bone tissue includes a facet joint;
the first member of the bone fixation device is a bone screw; and
the second member of the bone fixation device is a nut threadedly coupled to the bone screw.

10. A method, comprising:
coupling a bone fixation device to a distal end portion of an insertion tool such that distal movement of the bone fixation device along its longitudinal axis relative to the insertion tool is limited, the bone fixation device including a first member and a second member movably coupled to the first member;
advancing at least a portion of the first member of the bone fixation device into a bone tissue within a patient's body using the insertion tool; and
moving the second member of the bone fixation device relative to the first member of the bone fixation device after the advancing, the moving performed using the insertion tool.

11. The method of claim 10, wherein the coupling includes disposing a proximal end portion of the bone fixation device within a recess defined by the distal end portion of the insertion tool such that a retention member of the insertion tool is removably disposed within a groove defined by the proximal end portion of the bone fixation device.

12. The method of claim 10, wherein the coupling includes any one of magnetically coupling, adhesively coupling and mechanically coupling.

13. The method of claim 10, wherein the coupling includes coupling the bone fixation device to the distal end portion of an insertion tool such that rotation of the second member of the bone fixation device relative to the insertion tool is limited.

14. The method of claim 10, wherein the coupling includes threadedly coupling the insertion tool to the first member of the bone fixation device.

15. The method of claim 10, wherein the coupling includes threadedly coupling a shaft of the insertion tool within a recess defined by the first member of the bone fixation device.

16. The method of claim 10, wherein;
the advancing includes rotating a first shaft of the insertion tool such that at least the first member of the bone fixation device is threaded into the bone tissue; and
the moving includes rotating a second shaft of the insertion tool such that at least the second member of the bone fixation device rotates, the second shaft different from the first shaft.

17. The method of claim 10, further comprising:
defining a passageway within the bone tissue, the defining performed using the insertion tool after the coupling and before the advancing.

18. The method of claim 10, further comprising:
decoupling the bone fixation device from the distal end portion of the insertion tool after the moving.

19. The method of claim 10, further comprising:
decoupling the bone fixation device from the distal end portion of the insertion tool after the moving, the decoupling performed by rotating a shaft of the insertion tool within a recess defined by the first member of the bone fixation device.

20. The method of claim 10, further comprising:
actuating a portion of the insertion tool such that the bone fixation device is decoupled from the distal end portion of the insertion tool, the actuating performed after the moving.

21. The method of claim 10, wherein:
the bone tissue includes a facet joint;
the first member of the bone fixation device is a bone screw; and
the second member of the bone fixation device is a nut threadedly coupled to the bone screw.

22. A kit, comprising:
a bone fixation device including a bone screw and a nut threadedly coupled to the bone screw; and
an insertion tool configured to define a passageway within a bone tissue within a patient's body and insert at least a portion of the bone fixation device into the passageway of the bone tissue, the insertion tool including
a first shaft having a distal end portion removably coupled to the nut, the first shaft configured to rotate the nut about the bone screw,
a second shaft having a portion movably disposed within the first shaft, the second shaft having a distal end portion engaged with the bone screw, the second shaft configured to rotate the bone screw, and
a third shaft having a portion movably disposed within the second shaft, the third shaft configured to define the passageway within the bone tissue.

23. The kit of claim 22, wherein a portion of the insertion tool and the bone fixation device are configured to be inserted percutaneously into the patient's body.

24. The kit of claim 22, wherein the insertion tool is configured to define the passageway and insert the portion of the bone fixation device into the passageway without being removed from the patient's body.

25. The kit of claim 22, wherein the insertion tool is configured to define the passageway, insert the portion of the bone fixation device into the passageway, and rotate the nut about the bone screw without being removed from the patient's body.

26. The kit of claim 22, wherein the insertion tool further includes a sheath removably disposed about the bone fixation device and the distal end portion of the first shaft.

27. The kit of claim 22, wherein the insertion tool includes a fourth shaft movably disposed within the second shaft and about the third shaft, the fourth shaft configured to be removably coupled to the bone screw.
